SEQUENCE LISTING (5 sequences)

SEQ ID NO: 1 - protein, 24 aa, Artificial Sequence
  CoRNR1 peptide region of the NCoR1 protein
THRLITLADHICQIITQDFARNQV

SEQ ID NO: 2 - DNA, 22 bp, Artificial Sequence
  FLNA-A1191+Y1235 forward primer
ctcttcgctgacacccacatcc

SEQ ID NO: 3 - DNA, 22 bp, Artificial Sequence
  FLNA-A1191+Y1235 reverse primer
tccacactgaactcagtggtgg

SEQ ID NO: 4 - DNA, 22 bp, Artificial Sequence
  FLNA-G1578 forward primer
cccagaccgtcaattatgtgcc

SEQ ID NO: 5 - DNA, 22 bp, Artificial Sequence
  FLNA-G1578 reverse primer
gggatctcgtcaccaccgtact

(19) United States
(12) Patent Application Publication
(10) Pub. No.: US 2018/0298448 A1
(43) Pub. Date: Oct. 18, 2018

(54) METHOD AND KIT FOR PATHOLOGIC GRADING OF BREAST NEOPLASM

(71) Applicants: Bin Tean TEH, Singapore (SG); Boon Ooi Patrick TAN, Singapore (SG); Puay Hoon TAN, Singapore (SG)

(72) Inventors: Bin Tean TEH, Singapore (SG); Boon Ooi Patrick TAN, Singapore (SG); Puay Hoon TAN, Singapore (SG); Jing TAN, Singapore (SG); Choon Kiat ONG, Singapore (SG); Weng Khong LIM, Singapore (SG); Chuan Young Cedric NG, Singapore (SG)

(73) Assignees: Bin Tean TEH, Singapore (SG); Boon Ooi Patrick TAN, Singapore (SG); Puay Hoon TAN, Singapore (SG)

(21) Appl. No.: 15/544,964

(22) PCT Filed: Oct. 4, 2015
(86) PCT No.: PCT/SG2015/050368

§ 371 Date: Jul. 20, 2017

(30) Foreign Application Priority Data
Jan. 21, 2015 (SG) 10201500462U

(51) Int. Cl. C12Q 1/6886

(52) U.S. Cl. CPC C12Q 1/6886; C12Q 2600/156

(57) ABSTRACT

The present disclosure is a method comprising the steps of performing one or more nucleic-acid based assays to identify mutations present in the breast tissue acquired from the subject corresponding to a first test module and a second test module associated with detection of at least one predetermined mutation of one or more genes, wherein each test module is configured to provide a positive outcome corresponding to at least one predetermined mutation detected in the tissue or a negative outcome corresponding to absence of detectable predetermined mutation in the sample; and identifying the type of neoplasm of the breast tissue based upon the provided outcome of the both test modules. Preferably, the first test module is associated with detection of mutation in MED12 gene and/or RARA gene, while the second test module is associated with detection of mutation in FLNA gene, SETD2 gene and/or MLL2 gene.

### TECHNICAL FIELD

The present disclosure relates to a method capable of pathologic stratification or identifying type of neoplasm with respect to breast tissue of a subject. More particularly, the disclosed method identifies neoplasm type, stage or group by way of detecting or mapping mutations concurrently present in the breast tissue according to predetermined test modules associated with the mutations of interest. A kit being configured to enable the disclosed method is provided in the present disclosure as well.

### BACKGROUND

Fibroepithelial neoplasms of the breast are disease entities characterized by a biphasic proliferation of both epithelial and stromal components. Fibroepithelial breast tumors include fibroadenomas (FAs) and phyllodes tumors (PTs), the latter of which can be further subdivided into benign, borderline, and malignant grades based on their histological features¹. While FAs affect millions of women worldwide annually³, PTs occur at a lower frequency of approximately 1% or less of breast tumors and up to 7% of Asian breast cancers⁴. Compared to FAs, PTs have a later median age of onset (35 years vs 43 years), and a higher propensity for local recurrence, with distant metastasis also occurring in some malignant PTs⁵.

Previous studies have suggested that PTs and FAs may be highly related⁴. FA-like areas are not uncommonly encountered during histopathological examination of PTs, and some studies have proposed a clonal progression from FA to PT⁶⁻¹⁰. At the molecular level, frequent Mediator of RNA polymerase II transcription subunit 12 (MED12) exon 2 mutations have recently been observed in FAs and PTs^{2,11-14,} while gene expression and DNA methylation analyses have implicated genes such as HOXB13 and HMGA2 in PT development¹⁵⁻¹⁷. Higher rates of copy number alterations (CNAs) have been also associated with PTs of higher grade^{18,19}, and a recent study profiling a small number of PTs (n=15, five per grade) using a targeted cancer gene panel revealed recurrent mutations in tumour protein p53 (TP53) and singleton mutations in retinoblastoma protein (RB1) and Neurofibromin 1 (NF1) exclusively in higher-grade PTs¹¹. However, unlike breast carcinomas (BCs) whose comprehensive mutational landscapes have been extensively studied²⁰⁻²³, comparatively little is known about the genetic and molecular relationships linking different types of breast fibroepithelial lesions.

The diagnosis and classification of PTs often present challenges to pathologists, particularly in the distinction of benign PT from FA. Such classification can be of clinical importance in offering disease-specific treatment to patients suffering from FA, PT or BC.

### SUMMARY

The present disclosure aims to provide a method for grading, identifying or categorizing types of neoplasm relating to breast tissue of a subject. By having the type, stage or group of neoplasm correctly identified, the present disclosure facilitates disease-specific therapies towards the subject.

Another object of the present disclosure is to employ one or more nucleic-acid based assays in assisting the grading, identifying or categorizing of the neoplasm type relating to breast tissue of the subject. The disclosed method utilizing nucleic-acid based assays provides reliable results to serve as a supportive diagnostic tool in addition to physical examination of the specimen for neoplasm grading. Particularly, the disclosed method allows pathologists to substantially differentiate benign PT from FA.

Further object of the present disclosure is to offer a kit containing at least partly the essential reagents to facilitate the performance of the aforesaid one or more nucleic-acid based assays in generating the desired neoplasm grading. The disclosed kit can be of various embodiments to operate under different platforms of nucleic-acid based assays.

At least one of the preceding objects is met, in whole or in part, by the present disclosure, in which one of the embodiments of the present disclosure is a method for identifying type of neoplasm in a breast tissue of a subject comprising the steps of performing one or more nucleic-acid based assays to identify mutations present in the breast tissue acquired from the subject through a first test module and a second test module, each of the first and second test module being associated with detection of at least one predetermined mutation of one or more genes and configured to provide a positive outcome corresponding to at least one predetermined mutation detected in the tissue or a negative outcome corresponding to absence of detectable predetermined mutation in the sample, the first test module being associated with detection of mutation in MED12 gene and/or mutation in Retinoic acid receptor alpha (RARA) gene and the second test module being associated with detection of mutation in Filamin A alpha (FLNA) gene, mutation in SET domain containing 2 (SETD2) gene and/or mutation in mixed-lineage leukemia protein 2 (MLL2) gene; and identifying the type of neoplasm of the breast tissue based upon the provided outcome of the first and second test modules. Preferably, the type of neoplasm is regarded as fibroadenomas when the outcome of the first test module and the second test module are respectively positive and negative. Alternatively, the type of neoplasm is regarded as phyllodes tumor when the outcome of the first test module and the second test module are both positive. Also, the first test module can be further associated with detection of mutation in Telomerase reverse transcriptase (TERT) gene of the subject.

According to a number of the preferred embodiments, the step of performing one or more nucleic-acid based assays further comprises a third test module being associated with detection of mutation in NF1 gene, mutation in RB1 gene and/or mutation in phosphatidylinositol-4,5-bisphosphate 3-kinase catalytic subunit alpha (PIK3CA) gene. Preferably, the type of neoplasm is regarded as malignant phyllodes tumor when the outcome of the first test module, the second test module and the third test module are all positive.

According to a plurality of the preferred embodiments of the disclosed method, the mutation in MED12 gene is a splice site mutation located at position −8 of exon 2 of the MED12 gene, a missense mutation located at codon 44 of cDNA of the MED12 gene or a missense mutation located at codon 36 of cDNA of the MED12 gene.

According to more preferred embodiments, the mutation in RARA gene corresponds to or results in p.F286del, p.F287L, p.N299H, p.R394Q, p.L409del and/or p.G289R found in a polypeptide translated thereof from the RARA gene of the subject.

For several embodiments, the mutation in FLNA gene corresponds to p.A1191T, p.S1199L, p.P1244S, p. 1687-1688TV>M and/or p.S1186W found in a polypeptide translated thereof from the FLNA gene of the subject. These mutations to be detected relates particularly to missense mutation on the produced polypeptide.

For a number of embodiments, the mutation in SETD2 gene relates to p.R1674-1675EA>D, p.K1587fs, p.Q1545*, p.Y1605fs and/or p.F1651fs found in a polypeptide translatable thereof. The mutations found in SETD2 gene are generally relating to missense or somatic mutation.

In a plurality of embodiments, the mutation to be detected in TERT gene is preferably located at the promoter region. For instance, mutation located at −124 and/or −146 of the promoter region of the TERT gene leading to missense mutation.

In another aspect of the present disclosure, a kit for identifying type of neoplasm in a breast tissue of a subject is provided. Preferable, the kit comprises at least one platform capable of performing one or more nucleic-acid based assays to identify mutations present in the breast tissue acquired from the subject corresponding to a first test module and a second test module that each test module is associated with detection of at least one predetermined mutation of one or more genes, each test module being configured to provide a positive outcome corresponding to at least one predetermined mutation detected in the tissue or a negative outcome corresponding to absence of detectable predetermined mutation in the sample, the first test module being associated with detection of mutation in MED12 gene, TERT and/or mutation in RARA gene, the second test module being associated with detection of mutation in FLNA gene, mutation in SETD2 gene and/or mutation in MLL2 gene. Preferably, the test modules are configured to emit a detectable or visual signal corresponds to any positive outcome. The type of neoplasm is regarded as fibroadenomas when the outcome of the first test module and the second test module are respectively positive and negative. Alternatively, the type of neoplasm is regarded as benign phyllodes tumor when the outcome of the first test module and the second test module are both positive.

In one or more embodiments of the disclosed kit, the at least one platform further comprises a third test module being associated with detection of mutation in NF1 gene, mutation in RB1 gene and/or mutation in PIK3CA gene. With the inclusion of the third test module, the kit of the present disclosure can further regard, grade or identify the type of neoplasm as malignant phyllodes tumor when the outcome of the first test module, the second test module and the third test module are all positive.

For some embodiments, the breast tissue is stromal cells to be used with the disclosed kit for neoplasm grading or identification.

### BRIEF DESCRIPTION OF DRAWINGS

FIG. 1A is a combined graph showing distribution of recurrently mutated genes identified by targeted sequencing in 100 fibroepithelial tumours including 21 FAs and 79 PTs, with the centre graph indicating recurrently mutated genes grouped and dots to denote occurrence of the second mutation in the same patient, the left-sided bar graph to show the number of alterations and the adjacent numbers to indicate alteration frequency in the cohort, right-sided panel to show the frequency of mutations by subtype, and asterisk to indicate samples without matched normal;

FIG. 1B is a simple diagram to illustrate overview of key genetic alterations and pathways associated with each phase of the fibroepithelial tumour spectrum based on finding of the present disclosure;

FIG. 2A is a schematic depiction of mutations in MED12 with the frequency of each alteration being denoted in parentheses after its label from left to right MED, transcription mediator complex subunit Med12, MED12-LCEWAV, eukaryotic Mediator 12 subunit domain, MED12-PQL, eukaryotic Mediator 12 catenin-binding domain;

FIG. 2B is a schematic depiction of mutations in RARA that domain in RARA: NR_DBD (DNA-binding domain of retinoic acid receptor); NR_LBD (ligand binding domain of retinoic acid receptor);

FIG. 2C is a schematic depiction of mutations in FLNA that domain in FLNA: CH(Calponin homology domain) and IG_FLMN (Filamin-type immunoglobulin domains);

FIG. 2D is a schematic depiction of mutations in SETD2 that domain in SETD2: AWS (associated with SET domains), SET (Su(var)3-9, Enhancer-of-zeste, trithorax) domain, WW (WWP domain), and SRI (set2 Rbp1 interacting domain);

FIG. 2E is a schematic depiction of mutations in MLL2 that domain in MLL2: zf-HC (PHD-like zinc-binding domain), RING-finger (Really Interesting New Gene) domain, PHD (PHD zinc finger), HMG (High Mobility Group-box domain) FYRN (F/Y-rich N-terminus), FYRC (FY-rich domain at C-terminal region) SET (Su(var)3-9, Enhancer-of-zeste, Trithorax) domain;

FIG. 3 summarizes landscape of somatic mutations in phyllodes tumors in graph (A) indicating somatic mutation counts in FA and PT. *p<0.001 (B) showing frequency of mutations per case in 22 phyllodes tumors as expressed number of mutations per megabase (Mb) of covered target sequence, (c) mutational signature in 22 pairs PTs, and (d) showing copy number aberration counts of tend to harbor more CNAs compared to their lower grade counterparts. N.S., non significant. *p<0.05, **p<0.01, ***p<0.001;

FIG. 4A is a graph showing percentages of samples with somatic mutations in fibroepithelial tumours and solid tumors from TCGA samples identified through published data sets available from cBioPortal (numbers of sample per study in parentheses) with ACC being Adrenocortical Carcinoma and ccRCC being Kidney Renal Clear Cell Carcinoma;

FIG. 4B is graph showing expression level of RARA detected by qPCR in fibroepithelial tumours harbouring with wild type (17 cases) or mutant (13 cases) RARA;

FIG. 4C is a graph indicating that RARA mutant transcriptional activity is lower than that of wild-type RARA with HEK293 cells transfected with RARE Cignal reporter and expression plasmids containing empty vector, wild-type and mutant RARA cDNAs respectively that the transcriptional activity was measured in the absence and presence of RA stimulation (error bars=SD, n=3);

FIG. 4D is a graph showing results of Mammalian two-hybrid assays performed in HEK293 cells to evaluate interactions of the wild-type or mutant RARA with the nuclear co-repressor NCoR1 in the absence and presence of RA (error bars=SD, n=3);

FIG. 5A shows a schematic mapping of somatic mutations in FLNA in breast cancer using domain structure of the FLNA protein and the alterations identified in breast cancer from published data sets available from cBioPortal (TCGA) with CH as Calponin homology domain and IG-FLMN as Filamin-type immunoglobulin domains;

FIG. 5B shows the result of cDNA Sanger sequencing of FLNA variants in 3 fresh frozen PTs;

FIG. 6A are graphs revealing pattern of EGFR amplification in borderline (sample 1056) and malignant (sample 1076) PTs;

FIG. 6B is a representative image of MC staining of EGFR indicated the protein level and location of EGFR in borderline PT (sample 1056) and the image shows that EGFR protein is exclusively localized in stromal cells and absent from epithelial cells;

FIG. 7 shows comparison of the mutation spectra in FA, PT and BC based on targeted-sequencing analysis, as well as representative genes known to be significantly mutated in BCs (TP53, PIK3CA, MAP3K1, GATA3 and CDH1);

FIG. 8A is photomicrograph of Hematoxylin and eosin (H&E) stained section of Sample 1007 acquired by way of Laser Capture Microdissection (LCM), with S to denote Stromal and E as Epithelium;

FIG. 8B shows Sanger sequencing of MED12, RARA and BRCA1 in bulk tissue, epithelial and stromal compartments of the same stained sample provided in FIG. 8A that the sequencing results reveal mutations are exclusive to the stromal compartment;

FIG. 9 (A) is low magnification photomicrograph of the paraffinised tumor section with broad leafy stromal fronds protruding into clefted spaces lined by benign epithelium, (B) is medium magnification of the mild to moderately cellular stroma covered by benign bilayer epithelium, (C) is a photomicrograph showing permeative border with stromal cells percolating into adjacent fat, (D) illustrates stromal mitotic activity through mitoses, and (E) are Sanger sequencing of RB1 and EGFR of Sample 004 tumor and matched blood;

FIG. 10A is a photomicrograph showing H&E-stained section of concurrent FA and PT selected for macro-dissection from one patient in mutations that gain of cancer-associated genes are exclusively localized in PTs, with the bottommost photomicrographs being IHC-staining with EGFR highlighting the protein in PT area but not in FA-like area; and

FIG. 10B depicts spectrum of somatic mutations in concurrent and longitudinal FAs/PTs based upon histological subtypes of each patient reported at the upper panel and the bottom panels are schematic map to indicate the mutation categories identified.

### DETAILED DESCRIPTION

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the phrase “in embodiments” means in some embodiments but not necessarily in all embodiments.

As used herein, the terms “approximately” or “about”, in the context of concentrations of components, conditions, other measurement values, etc., means+/−5% of the stated value, or +/−4% of the stated value, or +/−3% of the stated value, or +/−2% of the stated value, or +/−1% of the stated value, or +/−0.5% of the stated value, or +/−0% of the stated value.

The term “polynucleotide” or “nucleic acid” as used herein designates mRNA, RNA, cRNA, cDNA or DNA. The term typically refers to oligonucleotides greater than 30 nucleotide residues in length.

The term “primer” used herein throughout the specification refers to an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. Primers can be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

The term “gene” as used herein may refer to a DNA sequence with functional significance. It can be a native nucleic acid sequence, or a recombinant nucleic acid sequences derived from natural source or synthetic construct. The term “gene” may also be used to refer to, for example and without limitation, a cDNA and/or an mRNA encoded by or derived from, directly or indirectly, genomic DNA sequence.

According to one major aspect of the present disclosure, a method for identifying type of neoplasm in a breast tissue of a subject is disclosed. Preferably, the disclosed method comprises the steps of performing one or more nucleic-acid based assays to identify mutations present in the breast tissue acquired from the subject corresponding to a first test module and a second test module that each test module is associated with detection of at least one predetermined mutation of one or more genes, each test module being configured to provide a positive outcome corresponding to at least one predetermined mutation detected in the tissue or a negative outcome corresponding to absence of detectable predetermined mutation in the sample, the first test module being associated with detection of mutation in MED12 gene, mutation in TERT gene and/or mutation in RARA gene, the second test module being associated with detection of mutation in FLNA gene, mutation in SETD2 gene and/or mutation in MLL2 gene; and identifying the type of neoplasm of the breast tissue based upon the provided outcome of the first and second test modules.

One ordinary skilled artisan shall appreciate the fact that at least part of the nucleic acid-based assay described herein can include also at least some universally known procedures or steps to complete the assay despite such procedures may not be completely detailed in this specification. For instance, part of the nucleic acid-based assay can involve the step of extracting or isolating deoxyribonucleic acid (DNA) and/or ribonucleic acid (RNA) materials containing genetic information relating to the subject or the tissue sampled of the subject using any method or commercial kit known in the field. Part of the nucleic acid-based assay, as in some of the preferred embodiments, may involve also conducting polymerase chain reaction (PCR) towards the isolated DNA or RNA materials in conjunction with predetermined thermal cycles and conditions to amplify gene or part of the gene to be analyzed in the test module for concluding the pathologic grading. These pre-treatments or processes can form part of the nucleic acid-based assay to finally lead to identification of the allele, mutations and/or genotype of the desired genes for neoplasm grading and categorizing. Pursuant to some preferred embodiments of the disclosed method, the nucleic-acid based assay is preferably performed to identify, detect and/or genotype potential mutations resided in one or more genes of the subject giving rise to neoplasm or cancer development. The nucleic acid-based assay of the present disclosure comprises sequencing the genes of interested. The sequencing can be performed onto polynucleotides amplified and/or duplicated from the DNA or RNA materials isolated from the breast tissue. More specifically, the sequencing approach implementable in the present disclosure to effect the mutation identification or detection can be Sanger sequencing and/or ultra-deep targeted amplicon sequencing, which is effective and capable of catering highly precise and reliable result in identifying the interested mutations setting forth in the test modules. The details of the Sanger sequencing and/or ultra-deep targeted amplicon sequencing are further elaborated in the examples incorporated hereafter. It is important for other skilled artisans to appreciate the fact that the disclosed method can be conducted utilizing other known sequencing equivalent or non-equivalent procedures or approaches to detect presence of the interested mutation in the analyzed polynucleotides and such modification shall not depart from the scope of the present disclosure. Other known processes implementable to identify or assist in identifying these mutations can be any one of, but not limited to, temperature gradient gel electrophoresis, capillary electrophoresis, amplification-refractory mutation system-polymerase chain reaction (ARMS-PCR), dynamic allele-specific hybridization (DASH), target capture for next generation sequencing (NGS), high-density oligonucleotide SNP arrays or Restriction fragment length polymorphism (RFLP). The present disclosure utilizes pattern, outcome or results generated from the test modules, with regard to the predetermined gene correlated to the given module, to grade the neoplasm stage or type of the tissue sample rather than mere resorting specific primers or a single platform to realize the grading or categorizing. Modification towards the primers or platform implementable to effect the disclosed method in neoplasm grading such as varying length or hybridizing location of the sequencing primers shall fall within the scope of the present disclosure.

As described in the foregoing, the first test module and the second test module are respectively associated with the detection of at least one mutation of one or more gene specifically correlated to the test module and provide an outcome applicable for subsequent grading the neoplasm stage or type of the tissue sampled. More specifically, in a number of preferred embodiments, the first test module is associated to the detection of mutations resided in the MED12 gene, TERT gene and/or RARA gene. More preferable, the first module is associated to the detection of more than one mutation relating to the MED12 gene, TERT gene and/or RARA gene. For instance, the mutation detectable for MED12 gene and being associated with the first test module can be any one of a splice site mutation located at position −8 of exon 2 of the MED12 gene, a missense mutation located at codon 44 of cDNA of the MED12 gene or a missense mutation located at codon 36 of cDNA of the MED12 gene. Likewise, detectable mutations for RARA gene associated with the first test module can be any one of missense mutations resulting p.F286del, p.F287L, p.N299H, p.R394Q, p.L409del and/or p.G289R in polypeptide translated thereof. Furthermore, the mutation to be detected in TERT gene is preferably located at the promoter region. For instance, mutation located at −124 and/or −146 of the promoter region of the TERT gene leading to missense mutation

According to a number of embodiments of the disclosed method, the second test module is preferably associated with detectable predetermined mutations resided in FLNA gene, SETD2 gene and/or MLL2 gene. More specifically, the one or more mutations to be detected for FLNA gene in association with the second test module generally gives rise to p.A1191T, p.S1199L, p.P1244S, p. 1687-1688TV>M and/or p.S1186W in a polypeptide translated thereof. Mutation relating to SETD2 gene and associating to the second test module is any one or combined mutations give rises to p.R1674-1675EA>D, p.K1587fs, p.Q1545*, p.Y1605fs and/or p.F1651fs translatable from the SETD2 gene. Similarly, mutations of MLL2 gene to be detect and associated with the second test module is any mutation generally causing inactivating mutation such as p.V5482fs, p.Q1139*, p.G2668fs, p.Q3814* and/or p.L3457fs found in a polypeptide encoded by the MLL2 gene. For few preferred embodiments, the second test module of the disclosed method can be further associated with mutations of other genes in addition to the FLNA gene, SETD2 gene and/or MLL2 gene. These extra genes with interested mutations being inferable or indicative of the breast neoplasm type or stage that can be associated to the second test module are BCL-6 corepressor protein (BCOR) gene and Mitogen-activated protein kinase kinase kinase 1 (MAP3K1) gene.

In some embodiments of the disclosed method, the second test module may be configured to detect presence of mutations in genes of the subject other than the FLNA gene, SETD2 gene and/or MLL2 gene. For example, the second test module can be arranged to discover inactivating mutations such as p.K460*, p.W534* and/or p.K175fs in B-Cell CLL/Lymphoma 6 corepressor (BCOR) gene of the subject, or somatic mutation like p.M312fs and/or p.Q409fs in Mitogen-Activated Protein Kinase Kinase Kinase 1 (MAP3K1) gene of the subject.

To better grade or identify the neoplasm stage of the sampled breast tissue, the performing one or more nucleic-acid based assays may further comprise a third test module being associated with detection of mutation in NF1 gene, mutation in RB1 gene and/or mutation in PIK3CA gene. With the aid of the third testing module, the disclosed method can further grade or identify the tissue sample, which has advance to the stage of borderline malignant or malignant phyllodes tumor. Preferably, mutations of NF1 gene being associated with the third test module are mutations relating to p.K1014*, p.R416* and/or p.D2283fs found in polypeptide translatable thereof. These mutations targeted in the third test module for NF1 gene are resulting in either nonsense mutation or frameshift mutation that leads to neoplasm development. Similarly, mutations of RB1 gene being associated with the third test module are mutation regarding p.Q504*, p.N316fs, and/or p.P796fs found in polypeptide translated thereof. These mutations of RB1 gene cause also either nonsense or frameshift mutation. For PIK3CA gene, the interested mutation associated with the third test module mainly relates to p.H1047R/L, which is a missense mutation.

Further embodiments of the disclosed method can have more mutations of other relevant genes discovered through the third test module besides mutation in NF1 gene, RB1 gene and/or PIK3CA gene. With more mutated sites covered, the disclosed method is able to offer higher accuracy to timely detect, diagnose, categorize, group or recognize various stage of neoplasm development in the subject. The third test module can be used to detect recurrent mutation associated to p.L62R in a polypeptide encoded by epidermal growth factor receptor (EGFR) gene, inframe deletion mutation associated to p.I33del found in polypeptide encoded by Phosphatase and Tensin Homolog (PTEN) gene, inactivating frameshift mutation associated to p. 294fs or p.C229fs found in polypeptide encoded by Tumor Protein P53 (TP53) gene, somatic mutation associated to p.W407R found in polypeptide encoded by Erb-B2 Receptor Tyrosine Kinase 4 (ERBB4) gene, and/or duplication of Insulin-Like Growth Factor 1 Receptor (IGF1R) gene in the subject.

In accordance with the preferred embodiments, the first, second and/or the third module is fashioned to yield a positive outcome as far as at least one predetermined mutation of the gene associated to that particular test module is detected and vice versa. For example, the first test module brings forth a positive outcome in response to at least one mutation detected in MED12 gene, TERT gene and/or RARA gene of the breast tissue of the subject. On the contrary, the first test module yields a negative outcome in the absence of any detectable predetermined mutations relating to MED12 gene, TERT gene and/or RARA gene. The like principle is applicable for the second and the third test module to realize the disclosed method in neoplasm grading and identification. In a number of the preferred embodiments, each mutation of the gene under consideration in a test module may be subjected to separate nucleic acid-based assay operating under different known principles or platform for detection or identification. For these embodiments, the nucleic-acid based assays for respective mutations of the involved gene may not be performed in a concurrent basis but rather the results generated from each of the assays are retrieved or collected to the associated test module to compute or yield an outcome thereof. The disclosed method may simultaneously run the like assays for detecting mutations or allele of genes respectively associated to different modules in a single operation of the same platform according to other preferred embodiments that the results of each analyzed mutation will be then pulled to associate with the predetermined modules to compute the outcome for subsequent neoplasm grading. In line with the aforesaid, the nucleic acid-based assays described herein are free from being tied to a single operating platform or mechanism though identifying the interested mutations of the relevant genes under one platform is preferable for cost and/or time saving.

As setting forth in the foregoing description, the present disclosed method effectively grades the type of neoplasm in response to the outcome computed, generated or signaled by the test module used. In accordance with a plurality of the preferred embodiments, the disclosed method regards the type of neoplasm of the sampled breast tissue as fibroadenomas when the outcome of the first test module and the second test module are respectively positive and negative. It was found by inventors of the present disclosure that biomarker related to early onset of FA can be linked to mutations detectable in MED12, TERT gene and/or RARA gene of the subject. Whilst, FA samples are generally free from any detectable mutations in those gene associated to the second test module such as FLNA, SETD2, MLL2, BCOR, MAP3K1. Similarly, the present disclosure also correlates the analyzed breast tissues as FA when the third module delivers a negative outcome, in addition to respective positive and negative outcome of the first and second test modules, indicating no substantial interested mutations can be detected in those genes in association to the third module.

The disclosed method may regard the type of neoplasm as phyllodes tumor when the outcome of the first test module and the second test module are both positive according to the preferred embodiments. Based upon test and experiments performed, the present disclosure recognizes that developed phyllodes tumor appears to possess mutations for genes associated with both first and second test modules. Particularly, the neoplasm type of the sampled breast tissues can be conveniently regarded as phyllodes tumor in line with presence of the considered mutations in FLNA, SETD2, MLL2, BCOR or MAP3K1 gene besides identified interest mutations in gene associated with the first test module.

In order to further differentiate or sub-grade the identified phyllodes tumor, the present disclosure, in some preferred embodiments, bring forth the third test module to detect mutations of extra genes of the subject in addition to those present in the first and second modules. Particularly, the type of neoplasm of the acquired breast tissue is regarded as malignant phyllodes tumor when the outcome of the first test module, the second test module and the third test module are all positive, meaning that the acquired sample carries at least one mutated gene in each of the test module. It is possible also the sample or subjected tested with positive outcome of a test module may harbor two or more mutations in one or more genes associated with that particular test module. On the other hand, the disclosed method preferably regards the type of neoplasm of the sampled breast tissue as benign phyllodes tumor when the outcome of the third test module is negative and outcomes of both first and second test modules are positive.

Another aspect of the present disclosure relates to a kit for identifying type of neoplasm in a breast tissue of a subject. Preferably, the kit comprises at least one platform capable of performing one or more nucleic-acid based assays to identify mutations present in the breast tissue acquired from the subject corresponding to a first test module and a second test module, each of the first and second test modules being associated with detection of at least one predetermined mutation of one or more genes and configured to provide a positive outcome corresponding to at least one predetermined mutation detected in the tissue or a negative outcome corresponding to absence of detectable predetermined mutation in the sample, the first test module being associated with detection of mutation in MED12 gene, TERT gene and/or mutation in RARA gene, the second test module being associated with detection of mutation in FLNA gene, mutation in SETD2 gene and/or mutation in MLL2 gene.

The at least one platform operable for the disclosed kit to identify or assist in identifying these mutations can be any one of, but not limited to, temperature gradient gel electrophoresis, capillary electrophoresis, amplification-refractory mutation system-polymerase chain reaction (ARMS-PCR), dynamic allele-specific hybridization (DASH), target capture for next generation sequencing (NGS), high-density oligonucleotide SNP arrays or Restriction fragment length polymorphism (RFLP). Referring to a number of the preferred embodiments of the disclosed kit, each mutation of the gene under consideration in the test module may be subjected to separate nucleic acid-based assay carried in at least one of the aforesaid platforms for detection or identification. In some embodiments, the nucleic-acid based assays for respective mutations of the involved gene may not be performed in a concurrent basis but rather the results generated from each of the assays are collected to the associated test module to compute or yield an outcome thereof. For other preferred embodiments, the disclosed kit can be used, at least be part of, to simultaneously run the like assays for detecting mutations or allele of genes respectively associated to different modules under a single platform that the results of each analyzed mutation will be then pulled to associate with the predetermined modules to compute the outcome for subsequent neoplasm grading.

In more preferred embodiments, the nucleic acid-based assay of the disclosed kit comprises sequencing the genes of interested. Sequencing can be performed onto polynucleotides amplified and/or duplicated from DNA or RNA materials isolated from the breast tissue, more preferably stromal or epithelial cells of the breast tissue. More specifically, the sequencing approach implementable in the present disclosure to effect the mutation identification or detection can be Sanger sequencing and/or ultra-deep targeted amplicon sequencing which is effective and capable of catering highly precise and reliable result in identifying the interested mutations setting forth in the test modules.

In line with the foregoing description, the first test module and the second test module of the disclosed kit are respectively associated with the detection of at least one mutation of one or more gene specifically correlated to the test module and provide an outcome applicable for subsequent grading the neoplasm stage or type of the tissue sampled. More specifically, in a number of preferred embodiments of the disclosed kit, the first test module is associated to the detection of mutations resided in the MED12 gene, TERT gene and/or RARA gene. More preferable, the first module is associated to the detection of more than one mutation relating to the MED12 gene, TERT gene and/or RARA gene. For instance, the mutation detectable for MED12 gene and being associated with the first test module can be any one of a splice site mutation located at position −8 of exon 2 of the MED12 gene, a missense mutation located at codon 44 of cDNA of the MED12 gene or a missense mutation located at codon 36 of cDNA of the MED12 gene. Likewise, detectable mutations for RARA gene associated with the first test module can be any one of missense mutations corresponds to p.F286del, p.F287L, p.N299H, p.R394Q, p.L409del and/or p.G289R found in a polypeptide translated thereof. For TERT gene, the mutation to be detected is preferably located at the promoter region, e.g. mutation located at −124 and/or −146 of the promoter region of the TERT gene that finally results in missense mutation

In accordance with some preferred embodiments, the second test module is preferably associated with detectable predetermined mutations resided in FLNA gene, SETD2 gene and/or MLL2 gene. More specifically, the one or more mutations to be detected for FLNA gene in association with the second test module is mutation corresponding to p.A1191T, p.S1199L, p.P1244S, p. 1687-1688TV>M and/or p.S1186W in a polypeptide translatable from the FLNA gene of the subject. Mutation relating to SETD2 gene and associating to the second test module is any one or combined mutations resulting in p.R1674-1675EA>D, p.K1587fs, p.Q1545*, p.Y1605fs and/or p.F1651fs found in a polypeptide produced thereof. Similarly, mutations of MLL2 gene to be detect and associated with the second test module is any one or combined mutations causing inactivating mutations like p.V5482fs, p.Q1139*, p.G2668fs, p.Q3814* and/or p.L3457fs found in a polypeptide encoded thereof. For few preferred embodiments, the second test module of the disclosed kit can be further associated with mutations of other genes in addition to the FLNA gene, SETD2 gene and/or MLL2 gene. These extra genes with interested mutations being inferable or indicative of the breast neoplasm type or stage that can be associated to the second test module are BCL-6 corepressor protein (BCOR) gene and Mitogen-activated protein kinase kinase kinase 1 (MAP3K1) gene.

The disclosed kit facilitates utilization of the pattern, outcome or results generated from the test modules, with regard to the predetermined and correlated gene, to grade the neoplasm stage or type of the tissue sample. Preferably, the type of neoplasm of the sampled breast tissue is regarded as fibroadenomas when the outcome of the first test module and the second test module are respectively positive and negative. Conversely, the neoplasm type of the tested breast tissue is regarded as benign phyllodes tumor when the outcome of the first test module and the second test module are both positive.

In more preferable embodiments, the disclosed kit can further comprise a third test module being associated with detection of mutation in NF1 gene, mutation in RB1 gene and/or mutation in PIK3CA gene. With the aid of the third testing module, the disclosed kit favors further grading or identification of neoplasm type relating to the tissue sample, which may have advanced to the stage of borderline malignant or malignant phyllodes tumor. Preferably, mutations of NF1 gene being associated with the third test module are mutations relating to p.K1014*, p.R416* and/or p.D2283fs found in polypeptide encoded thereof. Similarly, mutations of RB1 gene being associated with the third test module are mutations regarding p.Q504*, p.N316fs, and/or p.P796fs found in correspondingly encoded polypeptide. For PIK3CA gene, the interested mutations associated with the third test module generally relates to mutation resulting in p.H1047R/L of the polypeptide encoded. Accordingly, the type of neoplasm of the acquired breast tissue is regarded, through the disclosed kit, as malignant phyllodes tumor when the outcome of the first test module, the second test module and the third test module are all positive, meaning that the acquired sample carries at least one mutated gene in each of the test module. On the other hand, the disclosed kit preferably enables the user of the kit to regard the type of neoplasm of the sampled breast tissue as benign phyllodes tumor when the outcome of the third test module is negative and outcomes of both first and second test modules are positive.

To accelerate results generation from the disclosed kit, the test modules are preferably configured to emit a detectable or visual signal corresponds to any positive outcome and vice versa. As far as one interested mutation associated to a given module is identified, a machine or user readable signal will be produced to highlight a positive outcome obtained thereof. For example, the disclosed kit can adopt an embodiment in the form of DNA chip on which various polynucleotides anchored to readily hybridize with target gene fragments, potentially harboring the interested mutation, amplified from the sampled breast tissue. The test module can be a group of polynucleotides or a dedicated area on the chip. A discrete spot, attached with the polynucleotide designed specifically to hybridize only with the target gene fragment bearing the mutation of interest under stringent condition, on DNA chip and belong to a particular test module shall give rise to a signal readable by a microarray machine in the occurrence of a successful hybridization to notify and associate detection of the interested mutation to that particular test module for yielding a positive outcome.

The above described method and/or kit can cater supportive diagnosis in addition to conventional neoplasm grading or categorizing based on physical examination of the breast biopsy with or without further staining. The physician may have to conduct re-examination of the sampled tissues if there exist discrepancies between the results concluded from histologic examination and the disclosed method and/or kit. For instance, a sample being regarded as FA or benign PT with positive outcome in all three test modules shall be subjected to re-examination by the physician. It is clearly shown in the experiments of the present disclosure that FA or benign PT sample shall be clear of any mutations resided in genes associated to the third module of the disclosed method and/or kit. It is highly possible that the result of the physical or histologic examination is false negative. Health of the tested subject can be in jeopardy due to delay of treatment in view of the false result. The disclosed method and/or kit of the present disclosure offers extra mechanism to work against false negative or positive results arisen from subjective histologic examination, which is primarily relied upon experience of the physician performing the session.

The following example is intended to further illustrate the invention, without any intent for the invention to be limited to the specific embodiments described therein.

### Example 1

Fibroepithelial tumors were diagnosed and subtyped according to clinical features and histopathological examination of surgically excised tumors. All cases were histologically reviewed by at least 2 expert breast pathologists. Criteria for diagnosis and grading were based on recommendations of the WHO Classification of Tumours of the Breast¹. Briefly, phyllodes tumors were diagnosed when the fibroepithelial neoplasms showed an exaggerated intracanalicular pattern with leaf-like fronds accompanied by stromal hypercellularity. A benign phyllodes tumor was concluded when the lesion showed mild stromal cellularity with minimal nuclear atypia, pushing borders and mitoses of 4 or less per 10 high power fields, without stromal overgrowth. A diagnosis of malignant phyllodes tumor was rendered when there was marked stromal cellularity and atypia, presence of stromal overgrowth and permeative margins, with mitotic activity of 10 or more per 10 high power fields. Tumors with intermediate features were regarded as borderline. All 100 cases consisting of 21 FAs and 79 PTs were from fresh frozen tissue. Details of the samples employed in the present study are provided in Table 1 below. Of these, 69 cases had matching normal tissue. An additional five cases from FFPE (formalin-fixed paraffin embedded) slides, comprising concurrent (n=3) and longitudinal (n=2) cases were later included in the study.

TABLE 1
Clinical Characterisitcs of Fibroepithelial Tumor Pateitns

| | | | | Size of | | |
|---|---|---|---|---|---|---|
| | | Age | | Tumor | | Histological |
| No. | Specimen ID | (year) | Ethnicity | (mm) | Type of Surgery | Diagnosis |
| 1 | Sample001* | 42 | Chinese | 25 | Excision | FA |
| 2 | Sample002 | 21 | Chinese | 35 | Excision | FA |
| 3 | Sample003 | 38 | Malay | 25 | Excision | FA |
| 4 | Sample005 | 39 | Chinese | 25 | Excision | FA |
| 5 | Sample006 | 37 | Others | 50 | Excision | FA |
| 6 | Sample007 | 28 | Chinese | 40 | Excision | FA |
| 7 | Sample008 | 31 | Others | 80 | Excision | FA |
| 8 | Sample010 | 21 | Chinese | 40 | Excision | FA |
| 9 | Sample011 | 27 | Chinese | 40 | Excision | FA |
| 10 | Sample013* | 50 | Chinese | 70 | Wide Excision | FA |
| 11 | Sample014* | 25 | Indian | 30 | Excision | FA |
| 12 | Sample015* | 17 | Malay | 65 | Excision | FA |
| 13 | Sample016* | 42 | Indian | 37 | Excision | FA |
| 14 | Sample018 | 39 | Malay | 15 | Excision | FA |
| 15 | Sample019 | 34 | Chinese | 11 | Excision | FA |
| 16 | Sample020 | 31 | Chinese | 25 | Excision | FA |
| 17 | Sample021 | 32 | Chinese | 13 | Excision | FA |
| 18 | Sample022 | 25 | Chinese | 39 | Excision | FA |
| 19 | Sample023 | 24 | Chinese | 20 | Excision | FA |
| 20 | Sample024 | 36 | Chinese | 30 | Excision | FA |
| 21 | Sample025 | 58 | Chinese | 45 | Excision | FA |
| 22 | Sample1001 | 55 | Chinese | 180 | Excision | Benign PT |
| 23 | Sample1002 | 41 | Others | 140 | Excision | Benign PT |
| 24 | Sample1003 | 55 | Chinese | 120 | Excision | Benign PT |
| 25 | Sample1004 | 43 | Others | 63 | Wide Excision | Benign PT |
| 26 | Sample1005 | 22 | Chinese | 35 | Excision | Benign PT |
| 27 | Sample1006 | 39 | Indian | 650 | Excision | Benign PT |
| 28 | Sample1007 | 26 | Chinese | 90 | Excision | Benign PT |
| 29 | Sample1008 | 27 | Malay | 45 | Excision | Benign PT |
| 30 | Sample1009 | 36 | Malay | 67 | Excision | Benign PT |
| 31 | Sample1010 | 45 | Chinese | 80 | SMAC | Benign PT |
| 32 | Sample1011 | 38 | Chinese | 95 | Excision | Borderline PT |
| 33 | Sample1012 | 60 | Chinese | 28 | Excision | Borderline PT |
| 34 | Sample1013 | 52 | Indian | 20 | Wide Excision | Borderline PT |
| 35 | Sample1014 | 26 | Chinese | 48 | Excision | Borderline PT |
| 36 | Sample1015 | 68 | Chinese | 190 | Mastectomy | Borderline PT |
| 37 | Sample1016 | 52 | Others | 50 | Wide Excision | Borderline PT |
| 38 | Sample1017 | 79 | Chinese | 44 | SMAC | Borderline PT |
| 39 | Sample1018 | 62 | Malay | 32 | Wide Excision | Borderline PT |
| 40 | Sample1019 | 53 | Malay | 63 | Wide Excision | Malignant PT |
| 41 | Sample1020 | 45 | Malay | 220 | Mastectomy | Malignant PT |
| 42 | Sample1021 | 59 | Chinese | 80 | SMAC | Malignant PT |
| 43 | Sample1022 | 55 | Others | 200 | Total Mastectomy | Malignant PT |
| 44 | Sample1024* | 44 | Chinese | 50 | Mastectomy | Benign PT |
| 45 | Sample1025* | 41 | Indian | 80 | Excision | Benign PT |
| 46 | Sample1026 | 30 | Indian | 55 | Excision | Benign PT |
| 47 | Sample1027 | 31 | Malay | 60 | Excision | Benign PT |
| 48 | Sample1028 | 21 | Malay | 60 | Excision | Benign PT |
| 49 | Sample1029 | 39 | Indian | 40 | Excision | Benign PT |
| 50 | Sample1030 | 30 | Others | 35 | Excision | Benign PT |
| 51 | Sample1031 | 68 | Malay | 220 | SMAC | Benign PT |
| 52 | Sample1032 | 28 | Chinese | 25 | Excision | Benign PT |
| 53 | Sample1033 | 31 | Chinese | 50 | Excision | Benign PT |
| 54 | Sample1034 | 21 | Others | 60 | Excision | Benign PT |
| 55 | Sample1035 | 18 | Malay | 55 | Excision | Benign PT |
| 56 | Sample1036 | 55 | Chinese | 250 | Total Mastectomy | Benign PT |
| 57 | Sample1037 | 47 | Malay | 40 | Wide Excision | Benign PT |
| 58 | Sample1038 | 55 | Malay | 120 | Total Mastectomy | Benign PT |
| 59 | Sample1039* | 55 | Malay | 55 | SMAC | Benign PT |
| 60 | Sample1040* | 46 | Chinese | 220 | SMAC | Benign PT |
| 61 | Sample1041* | 55 | Chinese | 60 | Wide Excision | Benign PT |
| 62 | Sample1042* | 20 | Chinese | 48 | Excision | Benign PT |
| 63 | Sample1043* | 48 | Indian | 25 | Wide Excision | Benign PT |
| 64 | Sample1044* | 31 | Others | 95 | Wide Excision | Benign PT |
| 65 | Sample1045 | 22 | Malay | 28 | Excision | Benign PT |
| 66 | Sample1046 | 53 | Chinese | 53 | Excision | Benign PT |
| 67 | Sample1079 | 41 | Chinese | 40 | Excision | Benign PT |
| 68 | Sample1023 | 45 | Others | 90 | Mastectomy | Borderline PT |
| 69 | Sample1047* | 63 | Malay | 150 | Mastectomy | Borderline PT |
| 70 | Sample1048* | 51 | Chinese | 100 | Mastectomy | Borderline PT |
| 71 | Sample1049 | 62 | Chinese | 40 | Wide Excision | Borderline PT |
| 72 | Sample1050 | 64 | Malay | 180 | Wide Excision | Borderline PT |
| 73 | Sample1051 | 58 | Chinese | 50 | Wide Excision | Borderline PT |
| 74 | Sample1052 | 69 | Chinese | 80 | Mastectomy | Borderline PT |
| 75 | Sample1053 | 54 | Malay | 200 | Mastectomy | Borderline PT |
| 76 | Sample1054 | 47 | Chinese | 190 | SMAC | Borderline PT |
| 77 | Sample1055 | 57 | Chinese | 120 | SMAC | Borderline PT |
| 78 | Sample1056 | 47 | Malay | 120 | Excision | Borderline PT |
| 79 | Sample1057 | 44 | Chinese | 100 | SMAC | Borderline PT |
| 80 | Sample1058* | 61 | Chinese | 40 | Excision | Borderline PT |
| 81 | Sample1059* | 52 | Malay | 45 | Wide Excision | Borderline PT |
| 82 | Sample1060* | 47 | Chinese | 125 | Mastectomy | Borderline PT |
| 83 | Sample1061* | 55 | Malay | 250 | SMAC | Borderline PT |
| 84 | Sample1062* | 57 | Chinese | 82 | Mastectomy | Borderline PT |
| 85 | Sample1063* | 35 | Chinese | 190 | Mastectomy | Borderline PT |
| 86 | Sample1064* | 57 | Malay | 120 | SMAC | Borderline PT |
| 87 | Sample1065* | 55 | Malay | 85 | Wide Excision | Borderline PT |
| 88 | Sample1066* | 36 | Others | 250 | SMAC | Borderline PT |
| 89 | Sample1067* | 43 | Chinese | 30 | Excision | Borderline PT |
| 90 | Sample1068* | 37 | Others | 100 | Mastectomy | Borderline PT |
| 91 | Sample1069* | 58 | Chinese | 30 | Excision | Borderline PT |
| 92 | Sample1070 | 36 | Chinese | 50 | Excision | Borderline PT |
| 93 | Sample1071 | 51 | Chinese | 40 | Excision | Borderline PT |
| 94 | Sample1072 | 45 | Chinese | 45 | Wide Excision | Borderline PT |
| 95 | Sample1073* | 39 | Chinese | 50 | Mastectomy | Malignant PT |
| 96 | Sample1074 | 53 | Chinese | 250 | Mastectomy | Malignant PT |
| 97 | Sample1075* | 51 | Chinese | 135 | Mastectomy | Malignant PT |
| 98 | Sample1076 | 54 | Chinese | 50 | Total Mastectomy | Malignant PT |
| 99 | Sample1077* | 47 | Malay | 40 | Mastectomy | Malignant PT |
| 100 | Sample1078* | 41 | Chinese | 165 | Mastectomy | Malignant PT |

Tumors and whole-blood were obtained from patients undergoing surgical excision of fibroepithelial tumors with informed consent. Genomic DNA (gDNA) from fresh frozen tissue was extracted and purified using the Qiagen Blood and Cell Culture DNA kit. Genomic DNA yield and quality were determined using Picogreen™ fluorometric analysis as well as visual inspection of agarose gel electrophoresis images. For FFPE samples from concurrent or longitudinal fibroepithelial tumors, the Qiagen FFPE Tissue Kit was used.

### Example 2

Whole-exome sequencing was performed in 22 phyllodes tumors with matched tumor-normal pairs. Native genomic DNA was fragmented with the Covaris S2 (Covaris) system using recommended settings. Sequencing adaptor ligation was performed using the TruSeq Paired-End Genomic DNA kit (Illumina). For enrichment of coding sequences, we used the TruSeq Exome Enrichment kit (Illumina) according to the manufacturer's recommended protocol. Exome-enriched libraries were then sequenced on the 11lumina HiSeq 2000 instrument to generate 76 bp paired-end reads. Bioinformatics analysis, comprising sequence alignment, variant calling and identification of candidate somatic variants was performed as described in previous work⁴⁶. Variants were filtered to retain only those covered by at least 15 reads and having at least three variant reads. Furthermore, those with variant allele frequencies (VAFs) lower than 5% were excluded. Indels overlapping simple repeat regions were discarded. All remaining candidate variants were visually inspected in the IGV genome browser⁴⁷ to exclude likely germline mutations and sequencing artifacts. The synonymous mutations identified in the exome sequencing are included in Table 2 below.

TABLE 2
List of synonymous mutations identified from whole-exome sequencing of 22 cases of phyllodes.

| | | | | | | | | Allele | |
|---|---|---|---|---|---|---|---|---|---|
| Gene | Specimen | Transcript | Nucleotide | Nucleotide | Amino acid | Total | Variant | Freq | |
| Symbol | ID | ID | (genomic) | (cDNA) | (protein) | Depth | depth | (%) | Strand |
| ABCA8 | Sample1017 | CCDS11680.1 | g.chr17: 66872820 | c.4104 G > A | p.Q1368Q | 180 | 51 | 28.33 | − |
| | | | C > T | | | | | | |
| ADAMTS3 | Sample1018 | CCDS3553.1 | g.chr4: 73156608 | c.2895 C > T | p.P965P | 21 | 9 | 42.86 | − |
| | | | G > A | | | | | | |
| AKAP1 | Sample1010 | CCDS11594.1 | g.chr17: 55183962 | c.1137 T > C | p.S379S | 77 | 17 | 22.08 | + |
| | | | T > C | | | | | | |
| AKAP17A | Sample1016 | CCDS14116.1 | g.chrX: 1713051 | c.696 C > T | p.S232S | 185 | 35 | 18.92 | + |
| | | | C > T | | | | | | |
| C1orf106 | Sample1003 | CCDS44292.1 | g.chr1: 200869255 | c.204 G > A | p.A68A | 78 | 22 | 28.21 | + |
| | | | G > A | | | | | | |
| CAPN11 | Sample1020 | CCDS47436.1 | g.chr6: 44140712 | c.564 A > G | p.V188V | 86 | 23 | 26.74 | + |
| | | | A > G | | | | | | |
| CAPS2 | Sample1015 | CCDS9008.2 | g.chr12: 75692554 | c.1014 T > C | p.N256N | 33 | 14 | 42.42 | − |
| | | | A > G | | | | | | |
| CC2D2B | Sample1017 | CCDS53560.1 | g.chr10: 97776020 | c.471 T > C | p.F157F | 29 | 16 | 55.17 | + |
| | | | T > C | | | | | | |
| CCNE2 | Sample1018 | CCDS6264.1 | g.chr8: 95897751 | c.636 C > T | p.Y212Y | 37 | 12 | 32.43 | − |
| | | | G > A | | | | | | |
| CDH8 | Sample1004 | CCDS10802.1 | g.chr16: 61851565 | c.1095 C > T | p.R365R | 27 | 6 | 22.22 | − |
| | | | G > A | | | | | | |
| CDHR1 | Sample1012 | CCDS7372.1 | g.chr10: 85972079 | c.1698 G > A | p.L566L | 79 | 15 | 18.99 | + |
| | | | G > A | | | | | | |
| CHRNB4 | Sample1004 | CCDS58392.1 | g.chr15: 78927868 | c.117 T > C | p.R39R | 41 | 9 | 21.95 | − |
| | | | A > G | | | | | | |
| CHST2 | Sample1009 | CCDS3129.1 | g.chr3: 142839988 | c.330 G > A | p.P110P | 38 | 16 | 42.11 | + |
| | | | G > A | | | | | | |
| CKMT2 | Sample1010 | CCDS4053.1 | g.chr5: 80555049 | c.990 C > T | p.H330H | 46 | 11 | 23.91 | + |
| | | | C > T | | | | | | |
| CLMN | Sample1009 | CCDS9933.1 | g.chr14: 95669715 | c.1971 G > T | p.V657V | 29 | 3 | 10.34 | − |
| | | | C > A | | | | | | |
| COG7 | Sample1020 | CCDS10610.1 | g.chr16: 23453847 | c.555 G > A | p.E185E | 34 | 9 | 26.47 | − |
| | | | C > T | | | | | | |
| CREBRF | Sample1018 | CCDS34293.1 | g.chr5: 172517644 | c.462 T > G | p.L154L | 42 | 16 | 38.10 | + |
| | | | T > G | | | | | | |
| CST9 | Sample1009 | CCDS33450.1 | g.chr20: 23586496 | c.6 G > A | p.S2S | 48 | 15 | 31.25 | − |
| | | | C > T | | | | | | |
| CTSW | Sample1018 | CCDS8117.1 | g.chr11: 65650257 | c.627 G > C | p.L209L | 95 | 18 | 18.95 | + |
| | | | G > C | | | | | | |
| DCST1 | Sample1020 | CCDS1083.1 | g.chr1: 155015932 | c.1119 C > T | p.A348A | 292 | 202 | 69.18 | + |
| | | | C > T | | | | | | |
| DDX10 | Sample1009 | CCDS8342.1 | g.chr11: 108544223 | c.216 A > G | p.S72S | 20 | 4 | 20.00 | + |
| | | | A > G | | | | | | |
| DGKZ | Sample1022 | CCDS55757.1 | g.chr11: 46388493 | c.161 + 19123 | p.R229R | 71 | 24 | 33.80 | + |
| | | | G > A | G > A | | | | | |
| DMRTB1 | Sample1008 | CCDS581.1 | g.chr1: 53925399 | c.273 G > C | p.P91P | 18 | 3 | 16.67 | + |
| | | | G > C | | | | | | |
| DOCK2 | Sample1005 | CCDS4371.1 | g.chr5: 169145665 | c.2137 T > C | p.L713L | 29 | 9 | 31.03 | + |
| | | | T > C | | | | | | |
| FAM149B1 | Sample1007 | CCDS44435.1 | g.chr10: 74937664 | c.213 T > G | p.S71S | 22 | 7 | 31.82 | + |
| | | | T > G | | | | | | |
| FAM171A2 | Sample1002 | CCDS45701.1 | g.chr17: 42433160 | c.945 C > A | p.T315T | 147 | 32 | 21.77 | − |
| | | | G > T | | | | | | |
| HDAC6 | Sample1005 | CCDS14306.1 | g.chrX: 48663917 | c.384 C > T | p.C128C | 171 | 45 | 26.32 | + |
| | | | C > T | | | | | | |
| IL17RC | Sample1012 | CCDS46746.1 | g.chr3: 9975235 | c.2121 G > C | p.G677G | 30 | 8 | 26.67 | + |
| | | | G > C | | | | | | |
| ITPRIP | Sample1015 | CCDS7557.1 | g.chr10: 106074847 | c.963 C > T | p.F321F | 65 | 24 | 36.92 | − |
| | | | G > A | | | | | | |
| KCNQ3 | Sample1011 | CCDS56554.1 | g.chr8: 133186552 | c.618 G > A | p.T206T | 133 | 25 | 18.80 | − |
| | | | C > T | | | | | | |
| KIAA1524 | Sample1018 | CCDS33812.1 | g.chr3: 108285436 | c.1323 A > C | p.T441T | 34 | 15 | 44.12 | − |
| | | | T > G | | | | | | |
| LMBR1L | Sample1019 | CCDS8780.2 | g.chr12: 49491485 | c.1440 C > T | p.P460P | 16 | 8 | 50.00 | − |
| | | | G > A | | | | | | |
| LRRK2 | Sample1022 | CCDS31774.1 | g.chr12: 40699634 | c.3825 C > T | p.V1275V | 52 | 17 | 32.69 | + |
| | | | C > T | | | | | | |
| LUZP1 | Sample1019 | CCDS30628.1 | g.chr1: 23420374 | c.381 C > T | p.F127F | 60 | 18 | 30.00 | − |
| | | | G > A | | | | | | |
| MDFIC | Sample1006 | CCDS34737.1 | g.chr7: 114562510 | c.39 C > T | p.A13A | 72 | 15 | 20.83 | + |
| | | | C > T | | | | | | |
| MEP1B | Sample1018 | CCDS45846.1 | g.chr18: 29790543 | c.999 C > T | p.Y333Y | 34 | 13 | 38.24 | + |
| | | | C > T | | | | | | |
| MGA | Sample1018 | CCDS55960.1 | g.chr15: 42005387 | c.3123 G > A | p.R1041R | 37 | 8 | 21.62 | + |
| | | | G > A | | | | | | |
| MRVI1 | Sample1019 | CCDS44538.2 | g.chr11: 10650311 | c.612 C > T | p.V204V | 165 | 48 | 29.09 | − |
| | | | G > A | | | | | | |
| MSRB3 | Sample1017 | CCDS8973.1 | g.chr12: 65847599 | c.405 C > T | p.C135C | 59 | 15 | 25.42 | + |
| | | | C > T | | | | | | |
| MTMR2 | Sample1017 | CCDS8306.1 | g.chr11: 95568499 | c.1671 C > A | p.A629A | 15 | 4 | 26.67 | − |
| | | | G > T | | | | | | |
| MUC16 | Sample1011 | CCDS54212.1 | g.chr19: 9090324 | c.1491 A > G | p.T497T | 93 | 36 | 38.71 | − |
| | | | T > C | | | | | | |
| MYH2 | Sample1022 | CCDS11156.1 | g.chr17: 10429954 | c.4149 G > A | p.T1383T | 44 | 11 | 25.00 | − |
| | | | C > T | | | | | | |
| MYT1 | Sample1019 | CCDS13558.1 | g.chr20: 62848471 | c.1683 G > C | p.R561R | 17 | 4 | 23.53 | + |
| | | | G > C | | | | | | |
| N4BP2L1 | Sample1004 | CCDS9345.2 | g.chr13: 32981873 | c.216 C > T | p.F72F | 60 | 15 | 25.00 | − |
| | | | G > A | | | | | | |
| NANOS1 | Sample1015 | CCDS7607.1 | g.chr10: 120789991 | c.678 C > G | p.L226L | 27 | 12 | 44.44 | + |
| | | | C > G | | | | | | |
| ONECUT2 | Sample1011 | CCDS42440.1 | g.chr18: 55103254 | c.306 G > A | p.S102S | 95 | 41 | 43.16 | + |
| | | | G > A | | | | | | |
| OPN5 | Sample1015 | CCDS4923.1 | g.chr6: 47762990 | c.447 C > T | p.A149A | 35 | 8 | 22.86 | + |
| | | | C > T | | | | | | |
| OR5J2 | Sample1011 | CCDS31522.1 | g.chr11: 55944495 | c.402 A > T | p.V134V | 135 | 54 | 40.00 | + |
| | | | A > T | | | | | | |
| PAPPA2 | Sample1020 | CCDS41438.1 | g.chr1: 176664925 | c.2676 G > A | p.Q892Q | 305 | 67 | 21.97 | + |
| | | | G > A | | | | | | |
| PAPPA2 | Sample1007 | CCDS41438.1 | g.chr1: 176709306 | c.4125 C > T | p.D1375D | 50 | 7 | 14.00 | + |
| | | | C > T | | | | | | |
| PAX5 | Sample1004 | CCDS6607.1 | g.chr9: 36923359 | c.903 T > A | p.I193I | 22 | 6 | 27.27 | − |
| | | | A > T | | | | | | |
| PAX7 | Sample1005 | CCDS186.1 | g.chr1: 19027296 | c.936 C > A | p.T312T | 53 | 13 | 24.53 | + |
| | | | C > A | | | | | | |
| PCDHA4 | Sample1004 | CCDS54914.1 | g.chr5: 140188050 | c.2388 + 11113 | p.T426T | 641 | 113 | 17.63 | + |
| | | | C > T | C > T | | | | | |
| PCDHB12 | Sample1012 | CCDS4254.1 | g.chr5: 140590657 | c.2178 G > A | p.S726S | 568 | 141 | 24.82 | + |
| | | | G > A | | | | | | |
| PCDHGB4 | Sample1009 | CCDS54924.1 | g.chr5: 140769317 | c.2421 + 27194 | p.G622G | 1011 | 287 | 28.39 | + |
| | | | C > T | C > T | | | | | |
| PHACTR4 | Sample1017 | CCDS41294.1 | g.chr1: 28792909 | c.483 G > A | p.Q161Q | 66 | 21 | 31.82 | + |
| | | | G > A | | | | | | |
| PINK1 | Sample1001 | CCDS211.1 | g.chr1: 20960254 | c.213 C > T | p.R71R | 68 | 24 | 35.29 | + |
| | | | C > T | | | | | | |
| PITX1 | Sample1006 | CCDS4182.1 | g.chr5: 134364655 | c.759 C > T | p.L253L | 179 | 45 | 25.14 | − |
| | | | G > A | | | | | | |
| PLOD1 | Sample1001 | CCDS142.1 | g.chr1: 12017059 | c.729 C > T | p.N243N | 71 | 15 | 21.13 | + |
| | | | C > T | | | | | | |
| RAB4B | Sample1017 | CCDS33030.1 | g.chr19: 41289901 | c.351 C > T | p.I117I | 238 | 103 | 43.28 | + |
| | | | C > T | | | | | | |
| RBFOX1 | Sample1007 | CCDS10532.1 | g.chr16: 7629901 | c.453 G > A | p.P151P | 51 | 16 | 31.37 | + |
| | | | G > A | | | | | | |
| RFWD3 | Sample1018 | CCDS32486.1 | g.chr16: 74670422 | c.1248 C > T | p.G416G | 13 | 3 | 23.08 | − |
| | | | G > A | | | | | | |
| RMND5B | Sample1011 | CCDS4431.1 | g.chr5: 177574771 | c.1005 G > C | p.V322V | 112 | 49 | 43.75 | + |
| | | | G > C | | | | | | |
| RNF213 | Sample1004 | CCDS58606.1 | g.chr17: 78346350 | c.12567 C > T | p.Y4189Y | 41 | 14 | 34.15 | + |
| | | | C > T | | | | | | |
| SERPINB5 | Sample1015 | CCDS32839.1 | g.chr18: 61166370 | c.585 G > T | p.V195V | 17 | 4 | 23.53 | + |
| | | | G > T | | | | | | |
| SHROOM2 | Sample1004 | CCDS14135.1 | g.chrX: 9864507 | c.2559 C > T | p.N853N | 127 | 28 | 22.05 | + |
| | | | C > T | | | | | | |
| SLC46A2 | Sample1004 | CCDS6786.1 | g.chr9: 115652773 | c.189 G > A | p.R63R | 135 | 28 | 20.74 | − |
| | | | C > T | | | | | | |
| SLC9A2 | Sample1006 | CCDS2062.1 | g.chr2: 103274192 | c.459 C > T | p.A153A | 132 | 36 | 27.27 | + |
| | | | C > T | | | | | | |
| SPTBN1 | Sample1003 | CCDS33198.1 | g.chr2: 54880757 | c.5589 G > T | p.L1850L | 44 | 5 | 11.36 | + |
| | | | G > T | | | | | | |
| SPTBN5 | Sample1020 | NM_016642 | g.chr15: 42185218 | c.258 C > T | p.D86D | 65 | 26 | 40.00 | − |
| | | | G > A | | | | | | |
| SYNDIG1L | Sample1016 | CCDS41970.1 | g.chr14: 74876370 | c.78 G > A | p.P26P | 198 | 77 | 38.89 | − |
| | | | C > T | | | | | | |
| TAF4 | Sample1011 | CCDS33500.1 | g.chr20: 60581740 | c.2049 A > G | p.P683P | 60 | 30 | 50.00 | − |
| | | | T > C | | | | | | |
| TBX4 | Sample1003 | CCDS11629.1 | g.chr17: 59557478 | c.819 C > T | p.S273S | 26 | 7 | 26.92 | + |
| | | | C > T | | | | | | |
| TMUB2 | Sample1016 | CCDS11479.1 | g.chr17: 42266939 | c.525 C > T | p.T175T | 174 | 91 | 52.30 | + |
| | | | C > T | | | | | | |
| TPRX1 | Sample1022 | CCDS33066.1 | g.chr19: 48305782 | c.486 G > A | p.P162P | 27 | 7 | 25.93 | − |
| | | | C > T | | | | | | |
| TRPM3 | Sample1020 | CCDS6634.1 | g.chr9: 73254051 | c.1047 G > A | p.V502V | 61 | 16 | 26.23 | − |
| | | | C > T | | | | | | |
| VSIG10 | Sample1003 | CCDS44992.1 | g.chr12: 118506219 | c.1530 C > T | p.N510N | 52 | 17 | 32.69 | − |
| | | | G > A | | | | | | |
| WDR90 | Sample1020 | CCDS42092.1 | g.chr16: 702523 | c.1110 C > T | p.H370H | 189 | 72 | 38.10 | + |
| | | | C > T | | | | | | |
| ZFHX3 | Sample1015 | CCDS10908.1 | g.chr16: 72821612 | c.10563 C > T | p.G2607G | 34 | 5 | 14.71 | − |
| | | | G > A | | | | | | |
| ZFP69B | Sample1017 | CCDS452.2 | g.chr1: 40928610 | c.954 A > G | p.Q318Q | 157 | 42 | 26.75 | + |
| | | | A > G | | | | | | |
| ZNF467 | Sample1017 | CCDS5899.1 | g.chr7: 149462373 | c.1218 G > A | p.A406A | 203 | 41 | 20.20 | − |
| | | | C > T | | | | | | |
| ZNF804B | Sample1017 | CCDS5613.1 | g.chr7: 88963295 | c.999 T > C | p.D333D | 81 | 50 | 61.73 | + |
| | | | T > C | | | | | | |
| ZNF808 | Sample1020 | CCDS46167.1 | g.chr19: 53057402 | c.1233 A > G | P.Q411Q | 269 | 92 | 34.20 | + |
| | | | A > G | | | | | | |
| ZSCAN5B | Sample1004 | CCDS46203.1 | g.chr19: 56701298 | c.1386 C > T | p.S462S | 112 | 33 | 29.46 | − |
| | | | G > A | | | | | | |

PCR amplification was conducted with Platinum Taq Polymerase (Life Technologies). The PCR program included one cycle at 95° C. for 10 min, 35 cycles at 95° C. for 30 s, 58° C. for 30 s and 72° C. for 1 min and one cycle at 72° C. for 10 min. The BigDye Terminator v.3.1 kit (Applied Biosystems) was used for bidirectional sequencing on generated PCR amplicons, and products were fractionated using the ABI PRISM 3730 Genetic Analyzer (Applied Biosystems). Sequencing traces were aligned to reference sequences using Lasergene 10.1 (DNASTAR) and were visually analysed. The present disclosure selected 60 putative somatic mutations for Sanger validation (both tumor and normal sample) comprising mutations in recurrently mutated genes, cancer-associated genes and randomly selected genes. Of these, 54 mutations were successfully validated, 4 were found to be false positives and 2 failed to sequence, indicating a true positive rate of 90%. Validated mutations are highlighted with an asterisk in Table 3.

TABLE 3
List of candidate somatic mutations identified from whole-exome sequencing of 22 cases of phyllodes tumors

| | | | | | Variant | Total | Variant Allele | In | | |
|---|---|---|---|---|---|---|---|---|---|---|
| Gene | Publish | | Amino acid | | Read | Read | Frequency | COSMIC | | |
| Symbol | ID | Nucleotide (genomic) | (protein) | Mutation type | Depth | Depth | (%) | ? | PROVEAN | SIFT |
| A2M | Sample1 | g.chr12: 9260214C > T | p.G262E | Missense_ | 3 | 38 | 8 | No | Deleterious | Damaging |
| | 020 | | | Mutation | | | | | | |
| ABCA3 | Sample1 | g.chr16: 2328293G > A | p.H1572Y | Missense_ | 36 | 102 | 35 | No | Deleterious | Damaging |
| | 015 | | | Mutation | | | | | | |
| ABCB1 | Sample1 | g.chr7: 87229457_87229459del | p.14delK | In_Frame_Del | 14 | 28 | 50 | No | Deletion | Neutral |
| | 015 | TTC | | | | | | | | |
| ABCB4 | Sample1 | g.chr7: 87073059_87073060del | p.R383fs | Frame_Shift_Del | 11 | 29 | 38 | No | NA | NA |
| | 011 | CT | | | | | | | | |
| ABCB9 | Sample1 | g.chr12: 123419901_123419902 | p.V607fs | Frame_Shift_Del | 39 | 127 | 31 | No | NA | NA |
| | 011 | delCA | | | | | | | | |
| ABCC9 | Sample1 | g.chr12: 22001135G > A | p.R939W | Missense_ | 21 | 85 | 25 | No | Deleterious | Damaging |
| | 022 | | | Mutation | | | | | | |
| ABR | Sample1 | g.chr17: 986763_986764delTG | p.S166fs | Frame_Shift_Del | 13 | 39 | 33 | No | NA | NA |
| | 011 | | | | | | | | | |
| ACSF3 | Sample1 | g.chr16: 89178494_89178529del | Splice_Site | Splice_Site | 60 | 160 | 38 | No | NA | NA |
| | 011 | TCGTAGGTTTGGGAAAAGTTCTTAAGTTC | | | | | | | | |
| | | TGAAACG | | | | | | | | |
| ACSM3 | Sample1 | g.chr16: 20797427A > G | p.M391V | Missense_ | 3 | 28 | 11 | No | Deleterious | Damaging |
| | 017 | | | Mutation | | | | | | |
| ACTN1 | Sample1 | g.chr14: 69387836C > T | p.R76H | Missense_ | 7 | 21 | 33 | No | Deleterious | Damaging |
| | 020 | | | Mutation | | | | | | |
| ACVR1 | Sample1 | g.chr2: 158655996C > T | p.G4R | Missense_ | 9 | 18 | 50 | No | Neutral | Damaging |
| | 009 | | | Mutation | | | | | | |
| ADAM32 | Sample1 | g.chr8: 38975647T > C | p.I34T | Missense_ | 5 | 16 | 31 | No | Deleterious | Damaging |
| | 011 | | | Mutation | | | | | | |
| ADAMTS | Sample1 | g.chr16: 77387722delC | p.D508fs | Frame_Shift_Del | 38 | 66 | 58 | No | NA | NA |
| 18* | 019 | | | | | | | | | |
| ADAMTS | Sample1 | g.chr11: 130275962G > A | p.R721W | Missense_ | 51 | 157 | 32 | No | Deleterious | Damaging |
| 8 | 005 | | | Mutation | | | | | | |
| ADCY4 | Sample1 | g.chr14: 24795037T > C | p.E571G | Missense_ | 30 | 94 | 32 | No | Neutral | Tolerated |
| | 007 | | | Mutation | | | | | | |
| A2M | Sample1 | g.chr12: 9260214C > T | p.G262E | Missense_ | 3 | 38 | 8 | No | Deleterious | Damaging |
| | 020 | | | Mutation | | | | | | |
| ABCA3 | Sample1 | g.chr16: 2328293G > A | p.H1572Y | Missense_ | 36 | 102 | 35 | No | Deleterious | Damaging |
| | 015 | | | Mutation | | | | | | |
| ABCB1 | Sample1 | g.chr7: 87229457_87229459del | p.14delK | In_Frame_Del | 14 | 28 | 50 | No | Deletion | Neutral |
| | 015 | TTC | | | | | | | | |
| ABCB4 | Sample1 | g.chr7: 87073059_87073060del | p.R383fs | Frame_Shift_Del | 11 | 29 | 38 | No | NA | NA |
| | 011 | CT | | | | | | | | |
| ABCB9 | Sample1 | g.chr12: 123419901_123419902 | p.V607fs | Frame_Shift_Del | 39 | 127 | 31 | No | NA | NA |
| | 011 | delCA | | | | | | | | |
| ABCC9 | Sample1 | g.chr12: 22001135G > A | p.R939W | Missense_ | 21 | 85 | 25 | No | Deleterious | Damaging |
| | 022 | | | Mutation | | | | | | |
| ABR | Sample1 | g.chr17: 986763_986764delTG | p.S166fs | Frame_Shift_Del | 13 | 39 | 33 | No | NA | NA |
| | 011 | | | | | | | | | |
| ACSF3 | Sample1 | g.chr16: 89178494_89178529de; | Splice_Site | Splice_Site | 60 | 160 | 38 | No | NA | NA |
| | 011 | TCGTAGGTTTGGGAAAAGTTCTTAAGTTC | | | | | | | | |
| | | TGAAACG | | | | | | | | |
| ACSM3 | Sample1 | g.chr16: 20797427A > G | p.M391V | Missense_ | 3 | 28 | 11 | No | Deleterious | Damaging |
| | 017 | | | Mutation | | | | | | |
| ACTN1 | Sample1 | g.chr14: 69387836C > T | p.R76H | Missense_ | 7 | 21 | 33 | No | Deleterious | Damaging |
| | 020 | | | Mutation | | | | | | |
| ACVR1 | Sample1 | g.chr2: 158655996C > T | p.G4R | Missense_ | 9 | 18 | 50 | No | Neutral | Damaging |
| | 009 | | | Mutation | | | | | | |
| ADAM32 | Sample1 | g.chr8: 38975647T > C | p.I34T | Missense_ | 5 | 16 | 31 | No | Deleterious | Damaging |
| | 011 | | | Mutation | | | | | | |
| ADAMTS | Sample1 | g.chr16: 77387722delC | p.D508fs | Frame_Shift_Del | 38 | 66 | 58 | No | NA | NA |
| 18* | 019 | | | | | | | | | |
| ADAMTS | Sample1 | g.chr11: 130275962G > A | p.R721W | Missense_ | 51 | 157 | 32 | No | Deleterious | Damaging |
| 8 | 005 | | | Mutation | | | | | | |
| ADCY4 | Sample1 | g.chr14: 24795037T > C | p.E571G | Missense_ | 30 | 94 | 32 | No | Neutral | Tolerated |
| | 007 | | | Mutation | | | | | | |
| ADIPOQ | Sample1 | g.chr3: 186572218A > G | p.I154V | Missense_ | 34 | 84 | 40 | No | Neutral | Tolerated |
| | 018 | | | Mutation | | | | | | |
| AFTPH | Sample1 | g.chr2: 64780470_6478047insC | p.S622fs | Frame_Shift_Ins | 14 | 50 | 28 | No | NA | NA |
| | 013 | | | | | | | | | |
| AHDC1 | Sample1 | g.chr1: 27877033C > T | p.V532M | Missense_ | 30 | 128 | 23 | No | Nuetral | Tolerated |
| | 006 | | | Mutation | | | | | | |
| AHR | Sample1 | g.chr7: 17378879G > A | p.S477N | Missense_ | 14 | 54 | 26 | No | Neutral | Tolerated |
| | 002 | | | Mutation | | | | | | |
| AIFM3 | Sample1 | g.chr22: 21328426G > T | p.D144Y | Missense_ | 10 | 40 | 25 | No | Deleterious | Damaging |
| | 012 | | | Mutation | | | | | | |
| AKAP11 | Sample1 | g.chr13: 42875001C > G | p.Q707E | Missense_ | 11 | 41 | 27 | No | Neutral | Damaging |
| | 015 | | | Mutation | | | | | | |
| ALOX15 | Sample1 | g.chr17: 4542851_4542864del | p.R66fs | Frame_Shift_Del | 11 | 180 | 6 | No | NA | NA |
| | 010 | GGAGGTGCCGTTTG | | | | | | | | |
| AMBP | Sample1 | g.chr9: 116840480G > | p.L4F | Missense_ | 36 | 150 | 24 | No | Neutral | Damaging |
| | 007 | | | Mutation | | | | | | |
| ANKS1B | Sample1 | g.chr12: 100200391G > C | p.P154A | Missense_ | 6 | 27 | 22 | No | Deleterious | Tolerated |
| | 019 | | | Mutation | | | | | | |
| ANO2 | Sample1 | g.chr12: 5848527G > A | p.R460X | Nonsense_ | 12 | 36 | 33 | No | NA | NA |
| | 012 | | | Mutation | | | | | | |
| ANPEP | Sample1 | g.chr15: 90349528T > C | p.N96S | Missense_ | 67 | 253 | 26 | No | Neutral | Tolerated |
| | 007 | | | Mutation | | | | | | |
| ARHGAP2 | Sample1 | g.chr17: 36666590G > C | p.E1192D | Missense_ | 14 | 31 | 45 | No | Neutral | Damaging |
| 3 | 018 | | | Mutation | | | | | | |
| ARHGAP3 | Sample1 | g.chr19: 47491278G > A | p.G1287R | Missense_ | 10 | 54 | 19 | No | Deleterious | Damaging |
| 5 | 012 | | | Mutation | | | | | | |
| ARHGAP9 | Sample1 | g.chr12: 57869165_57869172del | p.P485fs | Frame_Shift_Del | 107 | 217 | 49 | No | NA | NA |
| | 017 | GCCTTCGG | | | | | | | | |
| ARHGEF40 | Sample1 | g.chr14: 21543514_21543515del | p.L492fs | Frame_Shift_Del | 24 | 59 | 41 | No | NA | NA |
| | 015 | CT | | | | | | | | |
| ASGR2 | Sample1 | g.chr17: 7004974G > A | p.R286C | Missense_ | 37 | 98 | 38 | No | Deleterious | Damaging |
| | 018 | | | Mutation | | | | | | |
| ASXL1* | Sample1 | g.chr20: 31022441_31022442ins | p.G643fs | Frame_Shift_Ins | 13 | 85 | 15 | Yes | NA | NA |
| | 015 | G | | | | | | | | |
| ATL2 | Sample1 | g.chr2: 38540302C > G | p.K265N | Missense_ | 3 | 19 | 16 | No | Deleterious | Tolerated |
| | 015 | | | Mutation | | | | | | |
| ATP1A1 | Sample1 | g.chr1: 116933028_116933029 | p.Q406fs | Frame_Shift_Del | 9 | 40 | 23 | No | NA | NA |
| | 011 | delAG | | | | | | | | |
| BCOR* | Sample1 | g.chrX:? 39933692C > A | p.A303S | Missense_ | 34 | 135 | 25 | No | Neutral | Tolerated |
| | 015 | | | Mutation | | | | | | |
| BCOR* | Sample1 | g.chrX: 39933221T > A | p.K460X | Nonsense_ | 38 | 104 | 37 | No | NA | NA |
| | 009 | | | Mutation | | | | | | |
| BCORL1 | Sample1 | g.chrX: 129149183_129149184 | p.I813fs | Frame_Shift_Ins | 50 | 121 | 41 | No | NA | NA |
| | 009 | insT | | | | | | | | |
| BRCA1* | Sample1 | g.chr17: 41243803T > G | p.T1249P | Missense_ | 14 | 545 | 26 | No | Neutral | Damaging |
| | 007 | | | Mutation | | | | | | |
| C11orf65 | Sample1 | g.chr11: 10827239A > C | p.S159R | Missense_ | 38 | 79 | 48 | No | Neutral | Tolerated |
| | 018 | | | Mutation | | | | | | |
| C14orf23 | Sample1 | g.chr14: 29261307_29261308 | p.K115fs | Frame_Shift_Ins | 26 | 26 | 100 | No | NA | NA |
| | 006 | insC | | | | | | | | |
| C16orf70 | Sample1 | g.chr16: 67174454C > T | p.S279L | Missense_ | 23 | 109 | 21 | No | Deleterious | Damaging |
| | 003 | | | Mutation | | | | | | |
| C19orf44 | Sample1 | g.chr19: 16620509delC | p.S450fs | Frame_Shift_Del | 31 | 108 | 29 | No | NA | NA |
| | 005 | | | | | | | | | |
| C1RL | Sample1 | g.chr12: 7254441G > T | p.D181E | Missense_ | 38 | 146 | 26 | No | Neutral | Tolerated |
| | 022 | | | Mutation | | | | | | |
| CACNA1C | Sample1 | g.chr12: 2783799C > G | p.P1655A | Missense_ | 11 | 33 | 33 | No | Deleterious | Damaging |
| | 015 | | | Mutation | | | | | | |
| CACNA1H | Sample1 | g.chr16: 1254123G > A | p.A706T | Missense_ | 40 | 125 | 32 | No | Neutral | Tolerated |
| | 018 | | | Mutation | | | | | | |
| CAPN6 | Sample1 | g.chrX: 110494819_110494820 | p.N284fs | Frame_Shift_Del | 13 | 33 | 39 | No | NA | NA |
| | 011 | delTT | | | | | | | | |
| CARM1 | Sample1 | g.chr19: 11015690A > G | p.K95R | Missense_ | 19 | 56 | 34 | No | Neutral | Tolerated |
| | 001 | | | Mutation | | | | | | |
| CCDC105 | Sample1 | g.chr19: 15131436C > T | p.T280M | Missense_ | 13 | 71 | 18 | No | Deleterious | Damaging |
| | 008 | | | Mutation | | | | | | |
| CCDC40 | Sample1 | g.chr17: 78069131_78069132 | p.R968fs | Frame_Shift_Del | 59 | 151 | 39 | No | NA | NA |
| | 011 | delAG | | | | | | | | |
| CCDC87 | Sample1 | g.chr11: 66359086G > T | p.N467K | Missense_ | 47 | 79 | 59 | No | Deleterious | Damaging |
| | 019 | | | Mutation | | | | | | |
| CD244 | Sample1 | g.chr1: 160811264T > C | p.K131E | Missense_ | 27 | 75 | 36 | No | Neutral | Damaging |
| | 011 | | | Mutation | | | | | | |
| CD36 | Sample1 | g.chr7: 80300370C > T | p.A299V | Missense_ | 7 | 19 | 37 | No | Neutral | Tolerated |
| | 001 | | | Mutation | | | | | | |
| CEP57L1 | Sample1 | g.chr6: 109475109A > G | p.E179G | Missense_ | 5 | 18 | 28 | No | Deleterious | Damaging |
| | 007 | | | Mutation | | | | | | |
| CEP95 | Sample1 | g.chr17: 62512894C > T | p.P141S | Missense_ | 3 | 24 | 13 | No | Neutral | Tolerated |
| | 020 | | | Mutation | | | | | | |
| CERCAM | Sample1 | g.chr9: 131193476A > G | p.N366S | Missense_ | 15 | 60 | 25 | No | Neutral | Tolerated |
| | 010 | | | Mutation | | | | | | |
| CHD4* | Sample1 | g.chr12: 6701695C > T | p.A938T | Missense_ | 24 | 65 | 37 | No | Neutral | Tolerated |
| | 009 | | | Mutation | | | | | | |
| CHD8* | Sample1 | g.chr14: 21883749C > T | p.R651Q | Missense_ | 26 | 76 | 34 | Yes | Deleterious | Damaging |
| | 016 | | | Mutation | | | | | | |
| CHL1 | Sample1 | g.chr3: 391164A > T | p.Y324F | Missense_ | 22 | 59 | 37 | No | Deleterious | Damaging |
| | 011 | | | Mutation | | | | | | |
| CHSY1 | Sample1 | g.chr15: 101775690C > G | p.G138A | Missense_ | 10 | 50 | 20 | No | Deleterious | Damaging |
| | 001 | | | Mutation | | | | | | |
| CLN3 | Sample1 | g.chr16: 28500652C > T | p.D61N | Missense_ | 21 | 51 | 41 | No | Deleterious | Damaging |
| | 004 | | | Mutation | | | | | | |
| CNOT3 | Sample1 | g.chr19: 54651974C > T | p.P329L | Missense_ | 46 | 120 | 38 | No | Neutral | Damaging |
| | 020 | | | Mutation | | | | | | |
| COG6 | Sample1 | g.chr13: 40253706_40253707del | p.Q191fs | Frame_Shift_Del | 20 | 89 | 22 | No | NA | NA |
| | 011 | AG | | | | | | | | |
| COL27A1* | Sample1 | g.chr9: 117047025delC | p.P1319fs | Frame_Shift_Del | 14 | 70 | 20 | No | NA | NA |
| | 003 | | | | | | | | | |
| COL6A3 | Sample1 | g.chr2: 238287314C > T | p.S821N | Missense_ | 17 | 28 | 61 | No | Neutral | Tolerated |
| | 019 | | | Mutation | | | | | | |
| COL6A6 | Sample1 | g.chr3: 130284165_130284166 | p.Q330fs | Frame_Shift_Del | 27 | 68 | 40 | No | NA | NA |
| | 011 | delAG | | | | | | | | |
| CPZ | Sample1 | g.chr4: 8605805C > T | p.T200M | Missense_ | 43 | 98 | 44 | No | Neutral | Damaging |
| | 013 | | | Mutation | | | | | | |
| CREB3 | Sample1 | g.chr9: 35736505_35736506del | p.C300fs | Frame_Shift_Del | 49 | 148 | 33 | No | NA | NA |
| | 011 | TG | | | | | | | | |
| CRELD2 | Sample1 | g.chr22: 50315936_50315973del | Splice_Site | Splice_Site | 58 | 111 | 52 | No | NA | NA |
| | 022 | CCTCAGCAGTCAGGACCGGCCTCTCCGAT | | | | | | | | |
| | | TCTTACCCG | | | | | | | | |
| CSNK1E | Sample1 | g.chr22: 38694894T > C | p.D261G | Missense_ | 14 | 49 | 29 | No | Deleterious | Damaging |
| | 003 | | | Mutation | | | | | | |
| CTCF | Sample1 | g.chr16: 67654643G > A | p.R377H | Missense_ | 31 | 71 | 44 | Yes | Deleterious | Damaging |
| | 016 | | | Mutation | | | | | | |
| CTSB | Sample1 | g.chr8: 11710887_11710888del | p.L26fs | Frame_Shift_Del | 57 | 151 | 38 | No | NA | NA |
| | 011 | AG | | | | | | | | |
| CUL7 | Sample1 | g.chr6: 43021566AG > C | p.L11V | Missense_ | 5 | 21 | 24 | No | Neutral | Damaging |
| | 015 | | | Mutation | | | | | | |
| DACT3 | Sample1 | g.chr19: 47152031C > A | p.G533V | Missense_ | 8 | 17 | 47 | No | Deleterious | Damaging |
| | 018 | | | Mutation | | | | | | |
| DAPK1 | Sample1 | g.chr9: 90262269C > T | p.T427I | Missense_ | 7 | 28 | 25 | No | Neutral | Tolerated |
| | 020 | | | Mutation | | | | | | |
| DARS | Sample1 | g.chr2: 13674299G > A | p.R15W | Missense_ | 59 | 136 | 43 | No | Neutral | Damaging |
| | 020 | | | Mutation | | | | | | |
| DBR1 | Sample1 | g.chr3: 137886001_137886002 | p.E212fs | Frame_Shift_Del | 14 | 33 | 42 | No | NA | NA |
| | 011 | delCT | | | | | | | | |
| DDX26B | Sample | g.chrX: 134683595C > A | p.S257R | Missense_ | 3 | 17 | 18 | No | Neutral | Tolerated |
| | 008 | | | Mutation | | | | | | |
| DENND4A | Sample1 | g.chr15: 660340171T > C | p.K305R | Missense_ | 3 | 31 | 10 | No | Neutral | Tolerated |
| | 016 | | | Mutation | | | | | | |
| DIEXF | Sample1 | g.chr1: 210024734C > T | p.A738V | Missense_ | 50 | 100 | 50 | No | Deleterious | Tolerated |
| | 019 | | | Mutation | | | | | | |
| DLAT | Sample1 | g.chr11: 111899638C > A | p.A210D | Missense_ | 4 | 24 | 17 | No | Neutral | Damaging |
| | 015 | | | Mutation | | | | | | |
| DNAH11 | Sample1 | g.chr7: 21630855C > T | p.T776M | Missense_ | 12 | 37 | 32 | Yes | Deleterious | Damaging |
| | 015 | | | Mutation | | | | | | |
| DNAH9 | Sample1 | g.chr17: 11774964A > G | p.N3368S | Missense_ | 24 | 74 | 32 | No | Neutral | Tolerated |
| | 017 | | | Mutation | | | | | | |
| DNAJB7 | Sample1 | g.chr22: 41257875_41257877del | p.41delE | In_Frame_Del | 29 | 120 | 24 | No | Deletion | Deleterious |
| | 011 | CTT | | | | | | | | |
| DNAJC13 | Sample1 | g.chr3: 132193779T > G | p.F765L | Missense_ | 3 | 21 | 14 | No | Deleterious | Tolerated |
| | 016 | | | Mutation | | | | | | |
| DNAJC6 | Sample1 | g.chr1: 65855042C > G | p.P376A | Missense_ | 49 | 114 | 43 | No | Deleterious | Tolerated |
| | 019 | | | Mutation | | | | | | |
| DRD1 | Sample1 | g.chr5: 174870020C > A | p.C28F | Missense_ | 16 | 92 | 17 | No | Deleterious | Damaging |
| | 003 | | | Mutation | | | | | | |
| DYX1C1 | Sample1 | g.chr5: 55731751C > T | p.R271K | Missense_ | 13 | 37 | 35 | No | Neutral | Tolerated |
| | 009 | | | Mutation | | | | | | |
| EDEM1 | Sample1 | g.chr3: 5248868C > G | p.D416E | Missense_ | 3 | 47 | 6 | No | Neutral | Tolerated |
| | 022 | | | Mutation | | | | | | |
| EGFR* | Sample1 | g.chr7: 55210075T > G | p.L62R | Missense_ | 10 | 37 | 27 | Yes | Neutral | Damaging |
| | 022 | | | Mutation | | | | | | |
| EHBP1L1 | Sample1 | g.chr11: 65353024G > A | p.D1299N | Missense_ | 7 | 26 | 27 | No | Deleterious | Damaging |
| | 018 | | | Mutation | | | | | | |
| EIF2S1 | Sample1 | g.chr14: 67831626C > G | p.L48V | Missense_ | 4 | 24 | 17 | No | Deleterious | Damaging |
| | 020 | | | Mutation | | | | | | |
| EIF4E1B | Sample1 | g.chr5: 176072180G > A | p.R137H | Missense_ | 43 | 183 | 23 | No | Deleterious | Damaging |
| | 001 | | | Mutation | | | | | | |
| EIF5 | Sample1 | g.chr14: 103803555A > C | p.N144H | Missense_ | 15 | 33 | 45 | No | Deleterious | Damaging |
| | 022 | | | Mutation | | | | | | |
| EMR2 | Sample1 | g.chr19: 14865840T > C | p.R506G | Missense_ | 21 | 64 | 33 | No | Neutral | Tolerated |
| | 011 | | | Mutation | | | | | | |
| EOGT | Sample1 | g.chr3: 69053547T > C | p.K201R | Missense_ | 19 | 42 | 45 | No | Neutral | Tolerated |
| | 009 | | | Mutation | | | | | | |
| ERBB4* | Sample1 | g.chr2: 212568899A > G | p.W407R | Missense_ | 10 | 42 | 24 | No | Deleterious | Damaging |
| | 014 | | | Mutation | | | | | | |
| EXPH5 | Sample1 | g.chr11: 108381510_108381511 | p.N1575fs | Frame_Shift_Del | 48 | 145 | 33 | No | NA | NA |
| | 011 | delTT | | | | | | | | |
| FAM135B | Sample1 | g.chr8: 139153543G > A | p.R1230W | Missense_ | 20 | 55 | 36 | No | Deleterious | Damaging |
| | 015 | | | Mutation | | | | | | |
| FAM58A* | Sample1 | g.chrX: 152858095C > A | p.D142Y | Missense_ | 26 | 51 | 51 | No | Deleterious | Damaging |
| | 011 | | | Mutation | | | | | | |
| FAT3 | Sample1 | g.chr11: 92088328G > A | p.R1017Q | Missense_ | 38 | 118 | 32 | No | Neutral | Damaging |
| | 018 | | | Mutation | | | | | | |
| FBXL18 | Sample1 | g.chr7: 5541530C > T | p.V124M | Missense_ | 24 | 68 | 35 | No | Neutral | Tolerated |
| | 018 | | | Mutation | | | | | | |
| FBXO5 | Sample1 | g.chr6: 153304004G > C | p.R31G | Missense_ | 20 | 67 | 30 | No | Neutral | Tolerated |
| | 020 | | | Mutation | | | | | | |
| FCAR | Sample1 | g.chr19: 55401207G > A | p.R281Q | Missense_ | 10 | 44 | 23 | No | Neutral | Tolerated |
| | 008 | | | Mutation | | | | | | |
| FCHO1 | Sample1 | g.chr19: 17873655C > T | p.R38W | Missense_ | 11 | 53 | 21 | No | Deleterious | Damaging |
| | 004 | | | Mutation | | | | | | |
| FERMT2 | Sample1 | g.chr14: 53345393_53345394del | p.P290fs | Frame_Shift_Del | 9 | 22 | 41 | No | NA | NA |
| | 011 | TC | | | | | | | | |
| FGD3 | Sample1 | g.chr9: 95780476G > T | p.R445M | Missense_ | 17 | 62 | 27 | No | Deleterious | Damaging |
| | 005 | | | Mutation | | | | | | |
| FGFBP1* | Sample1 | g.chr4: 15937592_15937593del | p.L221fs | Frame_Shift_Del | 23 | 132 | 17 | No | NA | NA |
| | 003 | AG | | | | | | | | |
| FGG | Sample1 | g.chr4: 155528074A > T | p.Y304X | Nonsense_ | 7 | 48 | 15 | No | NA | NA |
| | 003 | | | Mutation | | | | | | |
| FLNA* | Sample1 | g.chrX: 153588592C > T | p.A1191T | Missense_ | 26 | 95 | 27 | No | Deleterious | Damaging |
| | 002 | | | Mutation | | | | | | |
| FLNA* | Sample1 | g.chrX: 153588433G > A | p.P1244S | Missense_ | 41 | 211 | 19 | No | Deleterious | Damaging |
| | 014 | | | Mutation | | | | | | |
| FLNA* | Sample1 | g.chrX: 153586590C > A | p.G1578C | Missense_ | 42 | 157 | 27 | No | Deleterious | Damaging |
| | 015 | | | Mutation | | | | | | |
| FLNA* | Sample1 | g.chrX:153588460A > G | p.Y1235H | Missense_ | 95 | 267 | 36 | No | Deleterious | Damaging |
| | 020 | | | Mutation | | | | | | |
| FN1 | Sample1 | g.chr2: 216232664G > A | p.R2314X | Nonsense_ | 49 | 127 | 39 | Yes | NA | NA |
| | 020 | | | Mutation | | | | | | |
| FRMD5 | Sample1 | g.chr15: 44166164G > T | p.F544L | Missense_ | 31 | 91 | 34 | No | Neutral | Tolerated |
| | 010 | | | Mutation | | | | | | |
| GAK | Sample1 | g.chr4: 884326_884327delTG | p.Y358fs | Frame_Shift_Del | 18 | 45 | 40 | No | NA | NA |
| | 011 | | | | | | | | | |
| GAL3ST4 | Sample1 | g.chr7: 99757748G > A | p.R422C | Missense_ | 19 | 73 | 26 | Yes | Deleterious | Damaging |
| | 004 | | | Mutation | | | | | | |
| GATAD2B | Sample1 | g.chr1: 153789891C > T | p.R286H | Missense_ | 14 | 39 | 36 | No | Neutral | Damaging |
| | 016 | | | Mutation | | | | | | |
| GGNBP2 | Sample1 | g.chr17: 34935826G > T | p.D333Y | Missense_ | 13 | 50 | 26 | No | Deleterious | Damaging |
| | 015 | | | Mutation | | | | | | |
| GLG1 | Sample1 | g.chr16: 74640733G > T | p.P87Q | Missense_ | 26 | 159 | 16 | No | Neutral | Tolerated |
| | 006 | | | Mutation | | | | | | |
| GOLGB1 | Sample1 | g.chr3: 121416264C > T | p.E1031K | Missense_ | 27 | 127 | 21 | No | Neutral | Damaging |
| | 004 | | | Mutation | | | | | | |
| GPR87 | Sample1 | g.chr3: 151012291C > T | p.R248Q | Missense_ | 11 | 45 | 24 | Yes | Neutral | Damaging |
| | 007 | | | Mutation | | | | | | |
| GRINA | Sample1 | g.chr8: 145066702A > G | p.I298V | Missense_ | 75 | 260 | 29 | No | Neutral | Tolerated |
| | 011 | | | Mutation | | | | | | |
| GTF3A | Sample1 | g.chr13: 28009327_28009330del | Splice_Site | Slice_Site | 24 | 66 | 36 | No | NA | NA |
| | 011 | AAAG | | | | | | | | |
| H2BFWT | Sample1 | g.ch4X: 103268004G > A | p.R77C | Missense_ | 72 | 206 | 35 | Yes | Neutral | Tolerated |
| | 015 | | | Mutation | | | | | | |
| HIST2H2AC | Sample1 | g.chr1: 149858631G > A | p.R36H | Missense_ | 58 | 209 | 28 | No | Deleterious | Damaging |
| | 001 | | | Mutation | | | | | | |
| HNRNPM | Sample1 | g.chr19: 8528536_8528537delAT | p.H135fs | Frame_Shift_Del | 46 | 96 | 48 | No | NA | NA |
| | 019 | | | | | | | | | |
| HSD17B14 | Sample1 | g.chr19: 49337579C > G | p.G55A | Missense_ | 30 | 136 | 22 | No | Neutral | Tolerated |
| | 010 | | | Mutation | | | | | | |
| IGLON5 | Sample1 | g.chr19: 51830039A > G | p.E178G | Missense_ | 21 | 62 | 34 | No | Deleterious | Damaging |
| | 018 | | | Mutation | | | | | | |
| IKZF2 | Sample1 | g.chr2: 213872521C > A | p.G382C | Missense_ | 75 | 187 | 40 | No | Deleterious | Damaging |
| | 019 | | | Mutation | | | | | | |
| INPP4B | Sample1 | g.chr4: 143045802G > A | p.A611V | Missense_ | 10 | 22 | 45 | No | Neutral | Damaging |
| | 018 | | | Mutation | | | | | | |
| ITGA11 | Sample1 | g.chr15: 68643023T > C | p.N331S | Missense_ | 15 | 82 | 18 | No | Deleterious | Damaging |
| | 019 | | | Mutation | | | | | | |
| ITGB6 | Sample1 | g.chr2: 161052787T > C | p.K96E | Missense_ | 9 | 28 | 32 | No | Neutral | Tolerated |
| | 019 | | | Mutation | | | | | | |
| ITGB7 | Sample1 | g.chr12: 53585670_53585672del | p.763delK | In_Frame_Del | 34 | 119 | 29 | No | Deletion | Deleterious |
| | 011 | CTT | | | | | | | | |
| JAK2* | Sample1 | g.chr9: 5126787G > A | p.G1132E | Missense_ | 24 | 56 | 32 | No | Neutral | Damaging |
| | 011 | | | Mutation | | | | | | |
| KHDRBS1 | Sample1 | g.chr1: 32508212A > C | p.Y440S | Missense_ | 14 | 40 | 35 | No | Deleterious | Damaging |
| | 011 | | | Mutation | | | | | | |
| KIAA0100 | Sample1 | g.chr17: 26955366C > T | p.R1504Q | Missense_ | 44 | 67 | 66 | No | Neutral | Tolerated |
| | 017 | | | Mutation | | | | | | |
| KIAA1549 | Sample1 | g.chr7: 138597137T > C | p.N933S | Missense_ | 37 | 72 | 51 | No | Neutral | Tolerated |
| | 019 | | | Mutation | | | | | | |
| KIAA1731 | Sample1 | g.chr11: 93462600_93462603del | Splice_Site | Splice_Site | 21 | 73 | 29 | No | Deleterious | NA |
| | 011 | GTGA | | | | | | | | |
| KLHL18 | Sample1 | g.chr3: 47364180G > T | p.C128F | Missense_ | 4 | 15 | 27 | No | Deleterious | Damaging |
| | 004 | | | Mutation | | | | | | |
| KLRG2 | Sample1 | g.chr7: 139138324G > T | p.S296Y | Missense_ | 15 | 29 | 52 | No | Neutral | Damaging |
| | 016 | | | Mutation | | | | | | |
| KRT35 | Sample1 | g.chr17: 39637022C > T | p.G110S | Missense_ | 32 | 174 | 18 | No | Neutral | Tolerated |
| | 014 | | | Mutation | | | | | | |
| KRT81 | Sample1 | g.chr12: 52681800G > A | p.R290W | Missense_ | 33 | 92 | 36 | No | Deleterious | Damaging |
| | 016 | | | Mutation | | | | | | |
| L1CAM | Sample1 | g.chrX: 153136367C > T | p.G191D | Missense_ | 43 | 101 | 43 | No | Deleterious | Damaging |
| | 018 | | | Mutation | | | | | | |
| LAMA3 | Sample1 | g.chr18: 21416962C > A | p.A1001D | Missense_ | 3 | 39 | 8 | No | Deleterious | Damaging |
| | 009 | | | Mutation | | | | | | |
| LHB | Sample1 | g.chr19: 49519442_49519443del | p.P103fs | Frame_Shift_Del | 137 | 424 | 32 | No | NA | NA |
| | 011 | AG | | | | | | | | |
| LONP2 | Sample1 | g.chr16: 48311278G > A | p.R242H | Missense_ | 11 | 36 | 31 | Yes | Deleterious | Damaging |
| | 006 | | | Mutation | | | | | | |
| LPCAT1 | Sample1 | g.chr5: 1489895_1489896delTT | p.K191fs | Frame_Shift_Del | 17 | 52 | 33 | No | NA | NA |
| | 011 | | | | | | | | | |
| LRP1 | Sample1 | g.chr12: 57602905C > T | p.A4062V | Missense_ | 70 | 184 | 38 | No | Deleterious | Tolerated |
| | 016 | | | Mutation | | | | | | |
| LRP1B | Sample1 | g.chr2: 141473612A > G | p.S1985P | Missense_ | 3 | 18 | 17 | No | Deleterious | Damaging |
| | 019 | | | Mutation | | | | | | |
| LRP2 | Sample1 | g.chr2: 170101380G > A | p.R1085C | Missense_ | 8 | 28 | 29 | Yes | Deleterious | Damaging |
| | 011 | | | Mutation | | | | | | |
| LRTOMT | Sample1 | g.chr11: 71800199C > A | p.H24N | Missense_ | 3 | 46 | 7 | No | Neutral | Tolerated |
| | 009 | | | Mutation | | | | | | |
| LZTS2 | Sample1 | g.chr10: 102763885C > T | p.R344W | Missense_ | 84 | 108 | 78 | No | Deleterious | Damaging |
| | 017 | | | Mutation | | | | | | |
| MAGEF1 | Sample1 | g.chr3: 1844289084G > A | p.L176F | Missense_ | 22 | 64 | 34 | No | Neutral | Tolerated |
| | 011 | | | Mutation | | | | | | |
| MAGI3 | Sample1 | g.chr1: 114226044G > T | p.R1285L | Missense_ | 11 | 39 | 28 | No | Neutral | Damaging |
| | 015 | | | Mutation | | | | | | |
| MARK4 | Sample1 | g.chr19: 45805827_45805828ins | p.E707fs | Frame_Shift_Ins | 5 | 55 | 9 | No | NA | NA |
| | 001 | G | | | | | | | | |
| MAST1 | Sample1 | g.chr19: 12958197G > A | p.E141K | Missense_ | 84 | 180 | 47 | No | Deleterious | Tolerated |
| | 019 | | | Mutation | | | | | | |
| MAST4* | Sample1 | g.chr5: 66550553_66440554del | p.S929fs | Frame_Shift_Del | 22 | 51 | 43 | No | NA | NA |
| | 011 | TG | | | | | | | | |
| MATN4 | Sample1 | g.chr20: 43933326C > T | p.R62Q | Missense_ | 50 | 113 | 44 | No | Neutral | NA |
| | 020 | | | Mutation | | | | | | |
| MDC1 | Sample1 | g.chr6: 30679695_30679696del | p.E675fs | Frame_Shift_Del | 16 | 40 | 40 | No | NA | NA |
| | 011 | TC | | | | | | | | |
| MED12* | Sample1 | g.chrX: 70339234_70339257del | p.A38_ | In_Frame_Del | 7 | 32 | 22 | No | Deletion | Deleterious |
| | 015 | GGCCTTGAATGTAAAACAAGGTTT | F45del | | | | | | | |
| MED12* | Sample1 | g.chrX: 70339253G > T | p.G44C | Missense_ | 18 | 74 | 24 | Yes | Deleterious | Damaging |
| | 003 | | | Mutation | | | | | | |
| MED12* | Sample1 | g.chrX: 70339253G > A | p.G44S | Missense_ | 13 | 73 | 18 | Yes | Deleterious | Damaging |
| | 004 | | | Mutation | | | | | | |
| MED12* | Sample1 | g.chrX: 70339253G > T | p.G44C | Missense_ | 8 | 48 | 17 | Yes | Deleterious | Damaging |
| | 006 | | | Mutation | | | | | | |
| MED12* | Sample1 | g.chrX: 70339254G > T | p.G44V | Missense_ | 17 | 53 | 32 | Yes | Deleterious | Damaging |
| | 007 | | | Mutation | | | | | | |
| MED12* | Sample1 | g.chrX: 70339251A > C | p.Q43P | Missense_ | 11 | 58 | 19 | Yes | Deleterious | Damaging |
| | 008 | | | Mutation | | | | | | |
| MED12* | Sample1 | g.chrX: 70338240T > G | p.L36R | Missense_ | 14 | 52 | 27 | Yes | Deleterious | Damaging |
| | 010 | | | Mutation | | | | | | |
| MED12* | Sample1 | g.chrX: 70339254G > A | p.G44D | Missense_ | 26 | 34 | 76 | Yes | Deleterious | Damaging |
| | 016 | | | Mutation | | | | | | |
| MED12* | Sample1 | g.chrX: 70339253G > A | p.G44S | Missense_ | 16 | 54 | 30 | Yes | Deleterious | Damaging |
| | 020 | | | Mutation | | | | | | |
| MED12* | Sample1 | g.chrX: 70339254G > A | p.G44D | Missense_ | 22 | 71 | 31 | Yes | Deleterious | Damaging |
| | 022 | | | Mutation | | | | | | |
| MGA* | Sample1 | g.chr15: 42005646G > C | p.E1128Q | Missense_ | 9 | 24 | 38 | No | Neutral | Damaging |
| | 018 | | | Mutation | | | | | | |
| MIB2 | Sample1 | g.chr1: 1550850C > G | p.A4G | Missense_ | 10 | 18 | 56 | No | Neutral | Damaging |
| | 006 | | | Mutation | | | | | | |
| MID2 | Sample1 | g.chrX: 107167621_107167622 | p.Q495fs | Frame_Shift_Del | 19 | 57 | 33 | No | NA | NA |
| | 011 | delAG | | | | | | | | |
| MIDN | Sample1 | g.chr19: 1254357_1254358delCC | p.C192fs | Frame_Shift_Del | 29 | 91 | 32 | No | NA | NA |
| | 016 | | | | | | | | | |
| MLH3 | Sample1 | g.chr14: 75497276_75497277 | p.G1319fs | Frame_Shift_Del | 32 | 69 | 46 | No | NA | NA |
| | 011 | delTC | | | | | | | | |
| MLL2* | Sample1 | g.chr12: 49435199delG | p.P2118fs | Frame_Shift_Del | 8 | 26 | 31 | No | NA | NA |
| | 010 | | | | | | | | | |
| MLL2* | Sample1 | g.chr12: 49427051G > A | p.Q3813X | Nonsense_ | 43 | 95 | 45 | No | NA | NA |
| | 019 | | | Mutation | | | | | | |
| MLLT6 | Sample1 | g.chr17: 36863752G > A | p.C68Y | Missense_ | 17 | 71 | 24 | No | Deleterious | Damaging |
| | 004 | | | Mutation | | | | | | |
| MLXIPL | Sample1 | g.chr7: 73011949G > A | p.P389L | Missense_ | 16 | 47 | 34 | No | Neutral | Tolerated |
| | 004 | | | Mutation | | | | | | |
| MPG | Sample1 | g.chr16: 135747_135748delAG | p.R290fs | Frame_Shift_Del | 45 | 141 | 32 | No | NA | NA |
| | 011 | | | | | | | | | |
| MPI | Sample1 | g.chr15: 75189913A > T | p.M372L | Missense_ | 16 | 57 | 28 | No | Neutral | Tolerated |
| | 020 | | | Mutation | | | | | | |
| MPST* | Sample1 | g.chr22: 37420806C > T | p.R204X | Nonsense_ | 14 | 52 | 27 | No | NA | NA |
| | 022 | | | Mutation | | | | | | |
| MRPS5 | Sample1 | g.chr2: 95756252C > T | p.C316Y | Missense_ | 18 | 35 | 51 | No | Deleterious | Damaging |
| | 016 | | | Mutation | | | | | | |
| MS4A15 | Sample1 | g.chr11: 60535115G > T | p.W112L | Missense_ | 13 | 28 | 46 | No | Deleterious | Damaging |
| | 018 | | | Mutation | | | | | | |
| MUC16 | Sample1 | g.chr19: 9057237G > T | p.T10070N | Missense_ | 70 | 210 | 33 | No | Deleterious | Damaging |
| | 020 | | | Mutation | | | | | | |
| MYH14 | Sample1 | g.chr19: 50760622G > A | p.R704H | Missense_ | 16 | 94 | 17 | No | Deleterious | Damaging |
| | 015 | | | Mutation | | | | | | |
| MYO19B | Sample1 | g.chr22: 26422577G > A | p.V2213I | Missense_ | 42 | 99 | 42 | No | Neutral | Damaging |
| | 011 | | | Mutation | | | | | | |
| MYO5C | Sample1 | g.chr15: 52517719T > A | p.E1073V | Missense_ | 16 | 45 | 36 | No | Deleterious | Tolerated |
| | 020 | | | Mutation | | | | | | |
| MYOC | Sample1 | g.chr1: 171605163delA | p.Y473fs | Frame_Shift_Del | 45 | 169 | 27 | No | NA | NA |
| | 010 | | | | | | | | | |
| NACA | Sample1 | g.chr12: 57111535G > T | p.1260H | Missense_ | 3 | 25 | 12 | No | Neutral | Damaging |
| | 004 | | | Mutation | | | | | | |
| NCDN | Sample1 | g.chr1: 36026386A > T | p.K212X | Nonsense_ | 30 | 100 | 30 | No | NA | NA |
| | 013 | | | Mutation | | | | | | |
| NEFH | Sample1 | g.chr22: 29885581_29885604del | p.A652_ | In_Frame_Del | 117 | 289 | 40 | No | Deletion | Deleterious |
| | 017 | AGGCCAAGTCCCCAGAGAAGGAAG | E659del | | | | | | | |
| NEK9 | Sample1 | g.chr14: 75570684G > C | p.A531G | Missense_ | 12 | 32 | 38 | No | Neutral | Tolerated |
| | 011 | | | Mutation | | | | | | |
| NF1* | Sample1 | g.chr17: 29684022_29684023del | p.K2595fs | Frame_Shift_Del | 40 | 42 | 95 | Yes | NA | NA |
| | 019 | AA | | | | | | | | |
| NOSIP | Sample1 | g.chr19: 50063886_50063888del | p.21delK | In_Frame_Del | 14 | 66 | 21 | No | Deletion | Deleterious |
| | 004 | CTT | | | | | | | | |
| NR1H4 | Sample1 | g.chr12: 100886416G > A | Splice_Site | Splice_Site | 6 | 17 | 35 | No | NA | NA |
| | 004 | | | | | | | | | |
| NRXN2 | Sample1 | g.chr11: 64410197G > A | p.P27S | Missense_ | 3 | 34 | 9 | No | Neutral | Tolerated |
| | 008 | | | Mutation | | | | | | |
| NUDT5 | Sample1 | g.chr10: 12212728_12212730del | p.179delL | In_Frame_Del | 39 | 140 | 28 | No | Deletion | Deleterious |
| | 003 | GCA | | | | | | | | |
| OCRL | Sample1 | g.chrX: 128696586G > A | p.G356D | Missense_ | 9 | 24 | 38 | No | Deleterious | Damaging |
| | 018 | | | Mutation | | | | | | |
| OGT | Sample1 | g.chrX: 70775875C > G | p.N332K | Missense_ | 18 | 69 | 26 | No | Deleterious | Damaging |
| | 013 | | | Mutation | | | | | | |
| OR11L1 | Sample1 | g.chr1: 248005192G > T | p.P3T | Missense_ | 48 | 69 | 70 | No | Neutral | Tolerated |
| | 011 | | | Mutation | | | | | | |
| OR2AG1 | Sample1 | g.chr11: 6806896T > C | p.S210P | Missense_ | 49 | 229 | 21 | No | Neutral | Tolerated |
| | 001 | | | Mutation | | | | | | |
| OR4C13 | Sample1 | g.chr11: 49974047A > T | p.I25L | Missense_ | 15 | 31 | 48 | No | Neutral | Tolerated |
| | 007 | | | Mutation | | | | | | |
| OR5B12 | Sample1 | g.chr11: 58207105G > A | p.H174Y | Missense_ | 32 | 79 | 41 | No | Deleterious | Damaging |
| | 017 | | | Mutation | | | | | | |
| OR5D13 | Sample1 | g.chr11: 55541516C > A | p.C201X | Nonsense_ | 16 | 52 | 31 | No | NA | NA |
| | 001 | | | Mutation | | | | | | |
| OR5H15 | Sample1 | g.chr3: 97888212_97888213del | p.F223fs | Frame_Shift_Del | 68 | 170 | 40 | No | NA | NA |
| | 011 | CA | | | | | | | | |
| OSTN | Sample1 | g.chr3: 190930393A > T | p.K24N | Missense_ | 31 | 48 | 65 | No | Neutral | Damaging |
| | 019 | | | Mutation | | | | | | |
| P2RX1 | Sample1 | g.chr17: 3819429C > T | p.V31I | Missense_ | 6 | 26 | 23 | No | Neutral | Tolerated |
| | 004 | | | Mutation | | | | | | |
| P4HA2 | Sample1 | g.chr5: 131546091C > T | p.G199R | Missense_ | 11 | 53 | 21 | No | Neutral | Damaging |
| | 008 | | | Mutation | | | | | | |
| PAK7 | Sample1 | g.chr20: 9561027T > C | p.Y252C | Missense_ | 86 | 182 | 47 | No | Neutral | Tolerated |
| | 009 | | | Mutation | | | | | | |
| PCDH10 | Sample1 | g.chr4: 134073618G > T | p.A775S | Missense_ | 43 | 138 | 31 | No | Neutral | Tolerated |
| | 017 | | | Mutation | | | | | | |
| PCGF3 | Sample1 | g.chr4: 727587C > T | p.40S | Missense_ | 12 | 49 | 24 | No | Neutral | Tolerated |
| | 010 | | | Mutation | | | | | | |
| PCK2 | Sample1 | g.chr14: 24573136A > C | p.E629A | Missense_ | 20 | 51 | 39 | No | Deleterious | Damaging |
| | 016 | | | Mutation | | | | | | |
| PCLO* | Sample1 | g.chr7: 82581306C > A | p.G2988V | Missense_ | 19 | 97 | 20 | No | Deleterious | Damaging |
| | 002 | | | Mutation | | | | | | |
| PCNX | Sample1 | g.chr14: 71413792C > A | p.A105D | Missense_ | 4 | 20 | 20 | No | Neutral | Tolerated |
| | 006 | | | Mutation | | | | | | |
| PCNXL4* | Sample1 | g.chr14: 60582498_60582499del | p.S239fs | Frame_Shift_Del | 17 | 41 | 41 | No | NA | NA |
| | 011 | AG | | | | | | | | |
| PIK3CA* | Sample1 | g.chr3: 178952085A > T | p.H1047L | Missense_ | 19 | 37 | 51 | Yes | Neutral | Tolerated |
| | 011 | | | Mutation | | | | | | |
| PIK3CG* | Sample1 | g.chr7: 106509548G > A | p.M514I | Missense_ | 37 | 159 | 23 | No | Neutral | Damaging |
| | 022 | | | Mutation | | | | | | |
| PLEC | Sample1 | g.chr8: 144997935_144997936del | p.H2191fs | Frmae_Shift_Del | 73 | 276 | 26 | No | NA | NA |
| | 011 | GT | | | | | | | | |
| POLDIP3 | Sample1 | g.chr22: 42992248T > C | p.M253V | Missense_ | 16 | 49 | 33 | No | Neutral | Tolerated |
| | 004 | | | Mutation | | | | | | |
| POLQ | Sample1 | g.chr3: 121215763C > A | p.V724L | Missense_ | 4 | 19 | 21 | No | Neutral | Tolerated |
| | 017 | | | Mutation | | | | | | |
| PPARGC1A | Sample1 | g.chr4: 23833217C > G | p.G131A | Missense_ | 3 | 44 | 7 | No | Neutral | Tolerated |
| | 016 | | | Mutation | | | | | | |
| PPIL2 | Sample1 | g.chr22: 22049750C > T | p.R512W | Missense_ | 13 | 75 | 17 | No | Neutral | Tolerated |
| | 002 | | | Mutation | | | | | | |
| PPL | Sample1 | g.chr16: 4940333G > A | p.A722V | Missense_ | 18 | 61 | 30 | No | Neutral | Tolerated |
| | 003 | | | Mutation | | | | | | |
| PRDM5 | Sample1 | g.chr4: 121702298T > A | Splice_Site | Splice_Site | 10 | 30 | 33 | No | Neutral | Damaging |
| | 011 | | | | | | | | | |
| PRKDC | Sample1 | g.chr8: 48748950_48748951insC | p.Q2633fs | Frame_Shift_Ins | 5 | 90 | 5 | No | NA | NA |
| | 009 | | | | | | | | | |
| PRKRIR | Sample1 | g.chr11: 76063270_76063271del | p.V308fs | Frame_Shift_Del | 30 | 86 | 35 | No | NA | NA |
| | 011 | CA | | | | | | | | |
| PROC | Sample1 | g.chr2: 128186454C > T | p.R440C | Missense_ | 28 | 125 | 22 | No | Deleterious | NA |
| | 004 | | | Mutation | | | | | | |
| PSMD4 | Sample1 | g.chr1: 15123805_151238055del | p.G207fs | Frame_Shift_Del | 40 | 172 | 23 | No | NA | NA |
| | 011 | GAGTA | | | | | | | | |
| PTGER3 | Sample1 | g.chr1: 71512746C > G | p.R172P | Missense_ | 35 | 142 | 25 | No | Deleterious | Damaging |
| | 010 | | | Mutation | | | | | | |
| RALGPS2 | Sample1 | g.chr1: 178854324A > C | p.K340Q | Missense_ | 7 | 21 | 33 | No | Deleterious | Damaging |
| | 004 | | | Mutation | | | | | | |
| RARA* | Sample1 | g.chr17: 38512307_38512316del | p.M406fs | Frame_Shift_Del | 34 | 76 | 45 | No | NA | NA |
| | 001 | GCCGCCTCTC | | | | | | | | |
| RARA* | Sample1 | g.chr17: 38512312_38512313del | p.P408fs | Frame_Shift_Del | 21 | 91 | 23 | No | NA | NA |
| | 003 | CT | | | | | | | | |
| RARA* | Sample1 | g.chr17: 38510642A > G | p.N299S | Missense_ | 43 | 170 | 25 | No | Deleterious | Tolerated |
| | 007 | | | Mutation | | | | | | |
| RARA* | Sample1 | g.chr17: 38510611G > A | p.G289R | Missense_ | 50 | 157 | 32 | No | Deleterious | Damaging |
| | 015 | | | Mutation | | | | | | |
| RARA* | Sample1 | g.chr17: 38510560C > G | p.R272G | Missense_ | 46 | 142 | 32 | No | Deleterious | Damaging |
| | 022 | | | Mutation | | | | | | |
| RASAL3 | Sample1 | g.chr19: 15563988G > A | p.P867L | Missense_ | 46 | 114 | 40 | No | Neutral | Damaging |
| | 016 | | | Mutation | | | | | | |
| RB1* | Sample1 | g.chr13: 48955394C > T | p.Q504X | Nonsense_ | 20 | 20 | 100 | No | NA | NA |
| | 019 | | | Mutation | | | | | | |
| RBBP7 | Sample1 | g.chrX: 16871893C > T | p.G268S | Missense_ | 3 | 32 | 9 | No | Deleterious | Damaging |
| | 017 | | | Mutation | | | | | | |
| RBM15 | Sample1 | g.chr1: 110882625_110882628 | p.V200fs | Frame_Shift_Del | 11 | 46 | 24 | No | NA | NA |
| | 012 | delGTAA | | | | | | | | |
| RBM6* | Sample1 | g.chr3: 50095415C > T | p.R650C | Missense_ | 13 | 23 | 57 | No | Neutral | NA |
| | 011 | | | Mutation | | | | | | |
| REV1 | Sample1 | g.chr2: 100020912A > G | p.S1014P | Missense_ | 3 | 33 | 9 | No | Deleterious | Damaging |
| | 020 | | | Mutation | | | | | | |
| REXO2 | Sample1 | g.chr11: 114320578G > T | p.D199Y | Missense_ | 3 | 22 | 14 | No | Deleterious | Damaging |
| | 012 | | | Mutation | | | | | | |
| RMND1 | Sample1 | g.chr6: 151754342C > A | p.G213C | Missense_ | 4 | 22 | 18 | No | Deleterious | Damaging |
| | 007 | | | Mutation | | | | | | |
| RNF167 | Sample1 | g.chr17: 4848295_4848296delCT | p.P346fs | Frame_Shift_Del | 11 | 34 | 32 | No | NA | NA |
| | 012 | | | | | | | | | |
| RTFDC1 | Sample1 | g.chr20: 55092018T > A | p.S207T | Missense_ | 11 | 48 | 23 | No | Neutral | Tolerated |
| | 019 | | | Mutation | | | | | | |
| RYR3 | Sample1 | g.chr15: 33905440G > A | p.V741M | Missense_ | 16 | 95 | 17 | No | Deleterious | Damaging |
| | 001 | | | Mutation | | | | | | |
| SART3 | Sample1 | g.chr12: 108920274G > C | p.Q658E | Missense_ | 20 | 53 | 38 | No | Neutral | Tolerated |
| | 011 | | | Mutation | | | | | | |
| SNC9A | Sample1 | g.chr2: 167055456C > T | p.R1887H | Missense_ | 20 | 85 | 24 | No | Deleterious | Damaging |
| | 017 | | | Mutation | | | | | | |
| SETD2* | Sample1 | g.chr3: 47139458delC | p.R1710fs | Frame_Shift_Del | 7 | 25 | 28 | No | NA | NA |
| | 002 | | | | | | | | | |
| SETD2* | Sample1 | g.chr3: 47163092delT | p.S1012fs | Frame_Shift_Del | 10 | 43 | 23 | No | NA | NA |
| | 010 | | | | | | | | | |
| SETD2* | Sample1 | g.chr3: 47139504_47139505del | p.R1694fs | Frame_Shift_Del | 20 | 30 | 67 | No | NA | NA |
| | 011 | CT | | | | | | | | |
| SETD2* | Sample1 | g.chr3: 47139564_47139566del | p.1674delE | Frame_Shift_Del | 6 | 17 | 35 | No | Deletion | Deleterious |
| | 001 | CTT | | | | | | | | |
| SETD2* | Sample1 | g.chr3: 47125841_47125852del | p.L1807_ | Frame_Shift_Del | 20 | 22 | 91 | No | Deletion | Deleterious |
| | 016 | GGAATGGGCAAG | P1810del | | | | | | | |
| SFMBT1 | Sample1 | g.chr3: 52941188T > C | p.Q743R | Missense_ | 3 | 33 | 9 | No | Neutral | Tolerated |
| | 022 | | | Mutation | | | | | | |
| SFXN3 | Sample1 | g.chr10: 102798419C > T | p.A268V | Missense_ | 35 | 87 | 40 | No | Deleterious | Damaging |
| | 018 | | | Mutation | | | | | | |
| SHROOM4 | Sample1 | g.chrX: 50350758_50350759ins | p.1128Ins | Frame_Shift_Del | 11 | 44 | 25 | No | NA | NA |
| | 013 | TGCTGCTGCTGT | GGGG | | | | | | | |
| SIN3A | Sample1 | g.chr15: 75693216T > G | p.E531A | Missense_ | 12 | 50 | 24 | No | Neutral | |
| | 003 | | | Mutation | | | | | Tolerated | |
| SIRPB1* | Sample1 | g.chr20: 1552478C > A | p.R213S | Missense_ | 28 | 114 | 25 | No | Neutral | Tolerated |
| | 017 | | | Mutation | | | | | | |
| SKP1 | Sample1 | g.chr5: 133494242G > C | p.C120W | Missense_ | 11 | 27 | 41 | No | Deleterious | Damaging |
| | 016 | | | Mutation | | | | | | |
| SLC22A17 | Sample1 | g.chr14: 23821195_23821196del | p.Y76fs | Frame_Shift_Del | 24 | 84 | 29 | No | NA | NA |
| | 011 | TA | | | | | | | | |
| SLC25A35 | Sample1 | g.chr17: 8194233A > G | p.V219A | Missense_ | 28 | 106 | 26 | No | Deleterious | Damaging |
| | 012 | | | Mutation | | | | | | |
| SLC30A4 | Sample1 | g.chr15: 45814243_45814244del | p.E103fs | Frame_Shift_Del | 21 | 68 | 31 | No | NA | NA |
| | 011 | TC | | | | | | | | |
| SLC38A10 | Sample1 | g.chr17: 79219486_79219487del | p.L1077fs | Frame_Shift_Del | 34 | 75 | 45 | No | NA | NA |
| | 011 | AG | | | | | | | | |
| SLC4A4 | Sample1 | g.chr4: 72215752G > A | p.M171I | Missense_ | 15 | 35 | 43 | No | Neutral | Damaging |
| | 202 | | | Mutation | | | | | | |
| SLC5A10 | Sample1 | g.chr17: 18872378C > T | p.A156V | Missense_ | 44 | 113 | 39 | No | Deleterious | Damaging |
| | 003 | | | Mutation | | | | | | |
| SLC8A2 | Sample1 | g.chr19: 47960208G > T | p.A440E | Missense_ | 3 | 31 | 10 | No | Deleterious | Damaging |
| | 015 | | | Mutation | | | | | | |
| SLCO1B1 | Sample1 | g.chr12: 21392050C > G | p.A668G | Missense_ | 7 | 23 | 30 | No | Neutral | Tolerated |
| | 012 | | | Mutation | | | | | | |
| SLITRK2 | Sample1 | g.chrX: 144905888G > T | p.G649X | Nonsense_ | 25 | 73 | 34 | No | NA | NA |
| | 020 | | | Mutation | | | | | | |
| SMAD4* | Sample1 | g.chr18: 48573563G > C | p.E49D | Missense_ | 8 | 36 | 22 | No | Neutral | Tolerated |
| | 010 | | | Mutation | | | | | | |
| SMCR8 | Sample1 | g.chr17: 18225968_18225969del | p.L800fs | Frame_Shift_Del | 29 | 88 | 33 | No | NA | NA |
| | 011 | CT | | | | | | | | |
| SNAP25 | Sample1 | g.chr20: 10273821G > T | p.R59L | Missense_ | 3 | 21 | 14 | No | Deleterious | Damaging |
| | 001 | | | Mutation | | | | | | |
| SOAT1 | Sample1 | g.chr1: 179292615G > T | p.L54F | Missense_ | 20 | 51 | 39 | No | Neutral | Tolerated |
| | 013 | | | Mutation | | | | | | |
| SOCS5 | Sample1 | g.chr2: 46986105_46986106insG | p.R146fs | Frame_Shift_Del | 22 | 58 | 38 | No | NA | NA |
| | 003 | | | | | | | | | |
| SOC30 | Sample1 | g.chr5: 157078621C > T | p.V156I | Missense_ | 20 | 79 | 25 | No | Neutral | Damaging |
| | 003 | | | Mutation | | | | | | |
| SPATA31E1 | Sample1 | g.chr9: 90502016C > A | p.Q872K | Missense_ | 25 | 63 | 40 | No | Neutral | Damaging |
| | 011 | | | Mutation | | | | | | |
| SPR | Sample1 | g.chr2: 73114599C > G | p.T13S | Missense_ | 3 | 16 | 19 | No | Deleterious | Damaging |
| | 008 | | | Mutation | | | | | | |
| SPTBN4 | Sample1 | g.chr19: 41073626A > C | p.K2132Q | Missense_ | 3 | 35 | 9 | No | Neutral | Damaging |
| | 017 | | | Mutation | | | | | | |
| SRRM1 | Sample1 | g.chr1: 24979011_24979013del | p.272delE | Frame_Shift_Del | 13 | 45 | 29 | No | Deletion | Neutral |
| | 011 | AGG | | | | | | | | |
| SS18L1 | Sample1 | g.chr20: 60738637T > C | p.M227T | Missense_ | 16 | 56 | 29 | No | Neutral | Damaging |
| | 001 | | | Mutation | | | | | | |
| ST6GALNAC4 | Sample1 | g.chr9: 130670841_130670842del | p.S246fs | Frame_Shift_Del | 13 | 64 | 20 | No | NA | NA |
| | 019 | CT | | | | | | | | |
| STAT3* | Sample1 | g.chr17: 40481766_40481767del | Splice_Site | Splice_Site | 26 | 82 | 32 | No | NA | NA |
| | 011 | CT | | | | | | | | |
| SUPT16H | Sample1 | g.chr14: 21938052C > T | p.D163N | Missense_ | 10 | 41 | 24 | No | Deleterious | Damaging |
| | 012 | | | Mutation | | | | | | |
| SUPT5H | Sample1 | g.chr19: 39960802A > C | p.K473T | Missense_ | 41 | 91 | 45 | No | Deleterious | Tolerated |
| | 016 | | | Mutation | | | | | | |
| SYNE1 | Sample1 | g.chr6: 152774743C > T | p.R1002Q | Missense_ | 7 | 39 | 18 | Yes | Deleterious | Damaging |
| | 004 | | | Mutation | | | | | | |
| SYNJ1 | Sample1 | g.chr21: 34030132C > A | p.K785N | Missense_ | 8 | 42 | 19 | No | Deleterious | Damaging |
| | 004 | | | Mutation | | | | | | |
| SYTL5 | Sample1 | g.chrX: 37893165T > C | p.I8T | Missense_ | 12 | 43 | 28 | No | Deleterious | Damaging |
| | 019 | | | Mutation | | | | | | |
| TBL3 | Sample1 | g.chr16: 2024769_202477delTG | p.C129fs | Frame_Shift_Del | 57 | 135 | 42 | No | NA | NA |
| | 011 | | | | | | | | | |
| TCF7L2 | Sample1 | g.chr10: 114900944_114900945 | Splice_Site | Splice_Site | 32 | 76 | 42 | No | NA | NA |
| | 016 | insGA | | | | | | | | |
| TET3* | Sample1 | g.chr2: 74273547T > C | p.L33P | Missense_ | 27 | 84 | 32 | No | Neutral | Damaging |
| | 013 | | | Mutation | | | | | | |
| TMC1 | Sample1 | g.chr9: 75366786G > T | p.A186S | Missense_ | 9 | 26 | 35 | No | Deleterious | Damaging |
| | 002 | | | Mutation | | | | | | |
| TNS1 | Sample1 | g.chr2: 218762627_218762628 | p.R21fs | Frame_Shift_Del | 43 | 156 | 28 | No | NA | NA |
| | 011 | delCT | | | | | | | | |
| TP53* | Sample1 | g.chr17: 7577058_7577059delCC | p.G293fs | Frame_Shift_Del | 43 | 56 | 77 | Yes | NA | NA |
| | 019 | | | | | | | | | |
| TP53* | Sample1 | g.chr17: 7577594_7577595delAC | p.C229fs | Frame_Shift_Del | 7 | 68 | 10 | Yes | NA | NA |
| | 021 | | | | | | | | | |
| TRAPPC8 | Sample1 | g.chr18: 29437566T > C | p.E1042G | Missense_ | 11 | 44 | 25 | No | Deleterious | Tolerated |
| | 022 | | | Mutation | | | | | | |
| TRERF1 | Sample1 | g.chr6: 42231057C > A | p.V629L | Missense_ | 56 | 154 | 36 | No | Neutral | Damaging |
| | 019 | | | Mutation | | | | | | |
| TRIM23 | Sample1 | g.chr5: 64887305T > C | p.Y559C | Missense_ | 3 | 18 | 17 | No | Deleterious | Tolerated |
| | 007 | | | Mutation | | | | | | |
| TRIM52 | Sample1 | g.chr5: 180687359_180687361 | p.152delE | Frame_Shift_Del | 59 | 113 | 52 | No | Deletion | Deleterious |
| | 019 | delTTC | | | | | | | | |
| TRIM66 | Sample1 | g.chr11: 8642801_8642802delGT | p.T932fs | Frame_Shift_Del | 12 | 29 | 41 | No | NA | NA |
| | 011 | | | | | | | | | |
| TRIP4 | Sample1 | g.chr15: 64716244C > A | p.S458Y | Missense_ | 3 | 25 | 12 | No | Deleterious | Damaging |
| | 022 | | | Mutation | | | | | | |
| TRMT2B | Sample1 | g.chrX: 100290653_100290654 | p.V141fs | Frame_Shift_Del | 13 | 30 | 43 | No | NA | NA |
| | 018 | delCA | | | | | | | | |
| TRPM6 | Sample1 | g.chr9: 77431832C > T | p.A395T | Missense_ | 19 | 66 | 29 | No | Deleterious | Tolerated |
| | 006 | | | Mutation | | | | | | |
| TSEN2 | Sample1 | g.chr3: 12531387G > A | p.D30N | Missense_ | 15 | 64 | 23 | No | Deleterious | Tolerated |
| | 004 | | | Mutation | | | | | | |
| TSPAN13 | Sample1 | g.chr7: 16815912G > A | p.G47D | Missense_ | 8 | 27 | 30 | No | Deleterious | Damaging |
| | 015 | | | Mutation | | | | | | |
| TSPYL2 | Sample1 | g.chrX: 53114186C > A | p.P350T | Missense_ | 41 | 148 | 28 | No | Deleterious | Damaging |
| | 015 | | | Mutation | | | | | | |
| TTBK2 | Sample1 | g.chr15: 43044235G > A | p.S1070L | Missense_ | 14 | 47 | 30 | No | Neutral | Damaging |
| | 001 | | | Mutation | | | | | | |
| TTC19 | Sample1 | g.chr17: 15928476_15927477del | p.R274fs | Frame_Shift_Del | 9 | 26 | 35 | No | NA | NA |
| | 011 | AC | | | | | | | | |
| TTC31 | Sample1 | g.chr2: 74717177T > C | p.L52P | Missense_ | 47 | 120 | 39 | No | Neutral | Tolerated |
| | 011 | | | Mutation | | | | | | |
| TTI1 | Sample1 | g.chr20: 36634688_36634690del | p.805delT | Frame_Shift_Del | 27 | 68 | 40 | No | Deletion | Neutral |
| | 011 | GTG | | | | | | | | |

pNeutral

| | | | | | | | | | | |
|---|---|---|---|---|---|---|---|---|---|---|
| UBAP2 | Sample1 | g.chr9: 33943533C > A | p.V534L | Missense_ | 11 | 50 | 22 | No | Neutral | Tolerated |
| | 022 | | | Mutation | | | | | | |
| UGGT2 | Sample1 | g.chr13: 96543180A > G | p.F965S | Missense_ | 13 | 37 | 35 | No | Deleterious | Damaging |
| | 011 | | | Mutation | | | | | | |
| URB2 | Sample1 | g.chr1: 229771684G > A | p.E442K | Missense_ | 20 | 81 | 25 | No | Neutral | Damaging |
| | 011 | | | Mutation | | | | | | |
| VPS13D | Sample1 | g.chr1: 12336226C > T | p.R861X | Nonsense_ | 21 | 74 | 28 | No | NA | NA |
| | 019 | | | Mutation | | | | | | |
| VPS18 | Sample1 | g.chr15: 41192356delC | p.A447fs | Frame_Shift_Del | 20 | 86 | 23 | No | NA | NA |
| | 013 | | | | | | | | | |
| WDR35 | Sample1 | g.chr2: 20130295C > A | p.G1006C | Missense_ | 3 | 24 | 13 | No | Deleterious | Damaging |
| | 017 | | | Mutation | | | | | | |
| WDR43 | Sample1 | g.chr2: 29152457G > T | p.E440X | Nonsense_ | 4 | 17 | 24 | No | NA | NA |
| | 006 | | | Mutation | | | | | | |
| WDR48 | Sample1 | g.chr3: 39108-74T > C | p.W102R | Missense_ | 14 | 66 | 21 | No | Deleterious | Damaging |
| | 004 | | | Mutation | | | | | | |
| WDR63 | Sample1 | g.chr1: 85583502C > T | p.P626L | Missense_ | 4 | 23 | 17 | No | Deleterious | Tolerated |
| | 006 | | | Mutation | | | | | | |
| WDR70 | Sample1 | g.chr5: 37727012G > C | p.G581A | Missense_ | 3 | 34 | 9 | No | Deleterious | Damaging |
| | 009 | | | Mutation | | | | | | |
| XIRP2 | Sample1 | g.chr2: 168103963G > T | p.A2021S | Missense_ | 17 | 71 | 24 | Yes | Neutral | Damaging |
| | 011 | | | Mutation | | | | | | |
| XPR1 | Sample1 | g.chr1: 180775261C > T | p.R171C | Missense_ | 10 | 24 | 42 | No | Neutral | Tolerated |
| | 016 | | | Mutation | | | | | | |
| YEATS4 | Sample1 | g.chr12: 69783933A > C | p.E174A | Missense_ | 6 | 18 | 33 | No | Deleterious | Tolerated |
| | 010 | | | Mutation | | | | | | |
| ZBBX | Sample1 | g.chr3: 167000034G > T | p.S749Y | Missense_ | 8 | 15 | 53 | No | Neutral | Damaging |
| | 006 | | | Mutation | | | | | | |
| ZNF326 | Sample1 | g.chr1: 90486423C > T | p.A416V | Missense_ | 3 | 21 | 14 | No | Neutral | Tolerated |
| | 009 | | | Mutation | | | | | | |
| ZNF396 | Sample1 | g.chr18: 32953858_32953859del | p.E133fs | Frame_Shift_Del | 73 | 178 | 41 | No | NA | NA |
| | 011 | CT | | | | | | | | |
| ZNF608 | Sample1 | g.chr5: 123982981_123982983 | p.1032delK | Frame_Shift_Del | 50 | 145 | 34 | No | Deletion | Deleterious |
| | 011 | delCTT | | | | | | | | |
| ZNF687 | Sample1 | g.chr1: 15129489C > G | p.S241C | Missense_ | 51 | 106 | 48 | No | Neutral | Tolerated |
| | 019 | | | Mutation | | | | | | |
| ZNF704 | Sample1 | g.chr8: 81605271G > A | p.P98L | Missense_ | 5 | 32 | 16 | No | Deleterious | Damaging |
| | 001 | | | Mutation | | | | | | |
| ZNF711 | Sample1 | g.chrX: 84525751C > A | p.H401Q | Missense_ | 23 | 59 | 39 | No | Deleterious | Damaging |
| | 011 | | | Mutation | | | | | | |
| ZNF729 | Sample1 | g.chr19: 22498740A > G | p.K841E | Missense_ | 119 | 306 | 39 | No | Neutral | Tolerated |
| | 018 | | | Mutation | | | | | | |
| ZNF740 | Sample1 | g.chr19: 57955523T > C | p.M336T | Missense_ | 33 | 131 | 25 | No | Neutral | Tolerated |
| | 012 | | | Mutation | | | | | | |
| ZNF829 | Sample1 | g.chr19: 37382602C > T | p.S445N | Missense_ | 75 | 301 | 25 | No | Neutral | Tolerated |
| | 002 | | | Mutation | | | | | | |
| ZP4 | Sample1 | g.chr1: 23804607G > A | p.L488F | Missense_ | 8 | 35 | 23 | No | Neutral | Damaging |
| | 001 | | | Mutation | | | | | | |
| ZWILCH | Sample1 | g.chr15: 66825323G > T | Splice_Site | Splice_Site | 4 | 15 | 27 | No | Deleterious | Damaging |
| | 005 | | | | | | | | | |

Note:
Recurrent genes in bold and validated by Sanger sequencing
*Mutations validated by Sanger Sequencing

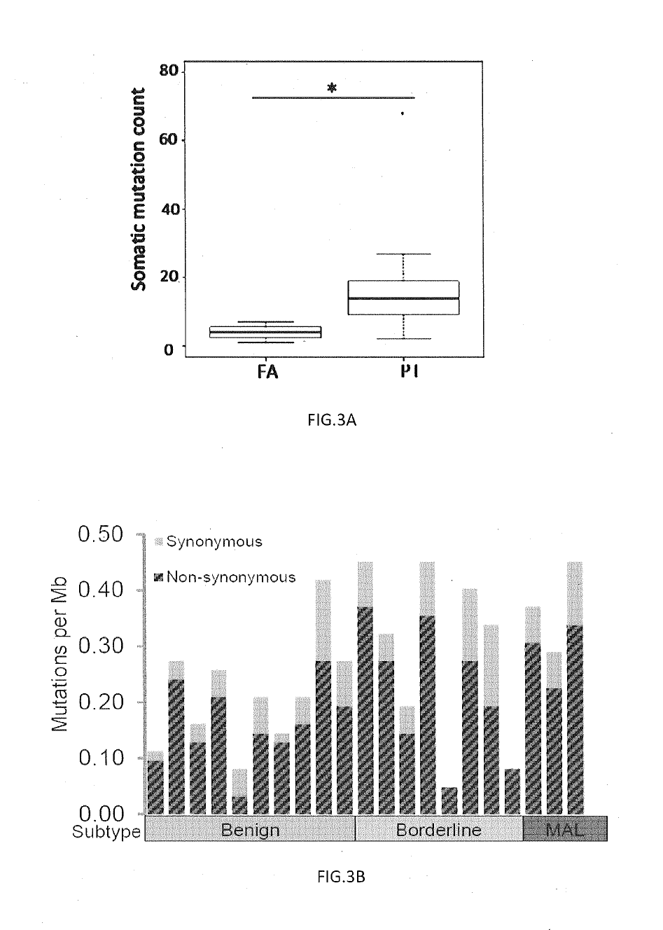

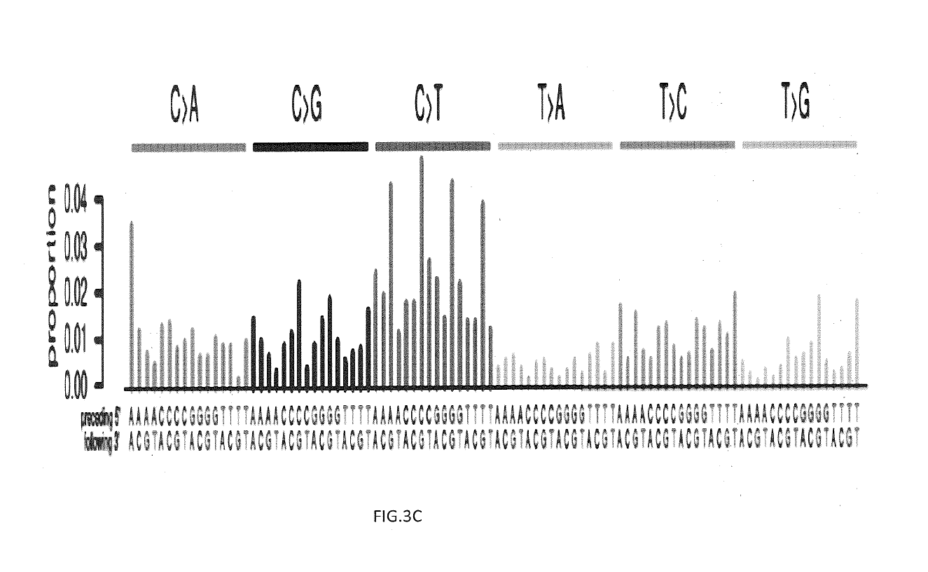

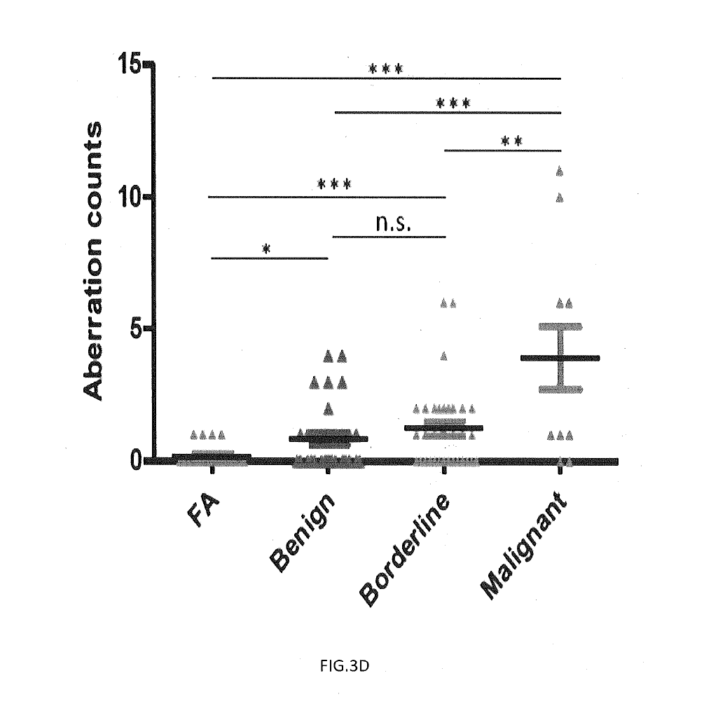

The present disclosure performed whole-exome sequencing of 22 matched tumor-normal pairs of PTs, including 10 benign, 8 borderline and 4 malignant PTs (Table 1). The PT exomes and matched normal samples were sequenced to a mean coverage of 66-fold, and on average 78% of bases were covered by at least 20 reads (Table 2). Inventors of the present disclosure identified a total of 333 non-synonymous or splice site somatic mutations in 310 genes. Sanger sequencing of recurrent (mutated in at least two cases) and singleton mutations of interest attained a 90% validation rate (Table 3). Despite a relatively lower depth of coverage compared to previous FA study (66× vs 124×) conducted by the inventors, the median count of non-silent somatic mutations/case in PTs was higher than in FAs (13 vs 5, p<0.001) as shown in FIG. 3A The relatively low mutation count in PTs is comparable to that of other mesenchymal tumors such as sarcomas and leiomyomas²⁴⁻²⁶ The mean non-synonymous mutation rate per megabase in PT was 0.192 (NS/S ratio=3.2, FIG. 3B) and the predominant mutation signature was that of C>T substitutions at NpCpG sites as indicated in FIG. 3C.

### Example 3

A panel of 50 selected genes (including recurrently mutated genes in the PT discovery cohort, genes mutated in FA², and also genes associated with breast cancer²⁰) was designed using the SureDesign tool (Agilent). Sequencing libraries were prepared from extracted DNA from 68 paired tumor-normal samples and 32 tumors using the SureSelect XT2 Target Enrichment System for Illumina Multiplexed Sequencing platform (Illumina) according to manufacturer's instructions. Target-enriched libraries were then sequenced on Illumina's HiSeq 2000 sequencing platform to generate 76 bp paired-end reads. For paired tumor-normal samples, analysis was performed as described in the exome sequencing analysis portion. In addition, due to higher sequencing coverage (samples had an average coverage in target region of at least 228×), the Strelka⁴⁸ (Illumina) somatic variant caller was used to identify low-allele frequency variants (at least 3%). All candidate variants were visually inspected in IGV to confirm that they are probably somatic. For patients where only the tumor sample was available, only variants in genes that were recurrently mutated among the paired tumor-normal samples were considered. The present disclosure also used a stricter variant allele frequency cut-off for the SNVs (at least 5%) and indels (at least 10%). Variants overlapping simple repeat regions were discarded, as were variants with dbSNP⁴⁹ (version 137) entries. Variants were also filtered against an in-house database containing germline variants identified in approximately 512 East Asian exomes to further remove likely germline polymorphisms. These variants were also visually inspected in IGV to exclude probable sequencing artefacts.

To validate the PT exome-sequencing data, inventors of the present disclosure performed targeted deep sequencing in a prevalence cohort of 100 fibroepithelial tumors (21 FAs, 34 benign, 35 borderline and 10 malignant PTs as shown in Table 1), which included 22 cases from the discovery cohort. The present disclosure sequenced a total of 50 genes, comprising recurrently mutated genes and singletons of interest in our discovery cohort, as well as genes previously reported to be mutated in FAs2 and BCs^{20-22,27}. The mean average coverage of target genes was 524× (minimum of 228×). The present disclosure acknowledges that the relatively low average depth of coverage (66×) attained in the exome sequencing of PTs is a limitation of the present study and may have resulted in under-calling of sequence variants. This is supported by further observation that 11 of 59 mutations identified by targeted sequencing (cut-off at 20% variant frequency) were missed by exome sequencing, resulting in a false-negative rate of 18.6%, likely due to low coverage. Also, due to the rarity of PTs and a relatively small discovery cohort, this study may have missed mutations occurring at low frequencies across patients as these would have been excluded from the targeted sequencing panel.

### Example 4

Copy number estimates for each of the genes in the targeted sequencing study were obtained using the OncoCNV²⁸. Briefly, depth of coverage information for each targeted regions were generated from BAM files and normalized against a pool of normal samples as well as GC content. Probe-level copy number estimates were then aggregated to obtain gene-level copy number estimates. Genes with copy number estimates less than 1.5 or more than 3 were considered to have copy number gains or losses. To identify copy number alterations (CNAs) and regions with LOH in our exome sequencing cohort, we used Control-FREEC²⁹.

To investigate the potential functions of the point mutations, we performed the mutation prediction algorithms, such as SIFT⁵⁰, Polyphen2⁵¹, CHASM⁵² and PROVEAN⁵³, respectively. The functional mutations were shown as damaging or probably damaging and deleterious in Table 4. Cancer-specific mutations were shown as drivers or passengers. Neutral mutations were shown as tolerated or benign.

Somatic mutations detected by targeted sequencing in 100 fibroepithelial tumors

| | | | | Total | Variant | | | | | | |
|---|---|---|---|---|---|---|---|---|---|---|---|
| Gene | | Nucleotide | | Read | Read | Variant Allele | In | | | | |
| Symbol | Speciman ID | (genomic) | Mutation type | Depth | Depth | Frequency | COSMIC‡ | SIFT | POLYPHEN2 | CHASM | PROVEAN |
| ADAMTS18 | Sample1019 | g.chr16: 77387 | Frameshift | 822 | 419 | 50.97 | No | NA | NA | NA | NA |
| | | 721 delC | | | | | | | | | |
| BCOR | Sample1074 | g.chrX: 399340 | Frameshift | 727 | 249 | 34.25 | No | NA | NA | NA | NA |
| | | 71delTGTT | | | | | | | | | |
| BCOR | Sample1015 | g.chrX: 399336 | Missense_ | 365 | 96 | 26.30 | No | Tolerated | Benign | passenger | Neutral |
| | | 92C > A | Mutation | | | | | | | | |
| BCOR | Sample1039* | g.chrX: 399114 | Missense_ | 1024 | 132 | 12.89 | No | Damaging | Probably | passenger | Deleterious |
| | | 14G > A | Mutation | | | | | | damaging | | |
| BCOR | Sample1009 | g.chrX: 399332 | Nonsense_ | 779 | 297 | 38.13 | No | NA | NA | NA | NA |
| | | 21T > A | Mutation | | | | | | | | |
| BCOR | Sample1068* | g.chrX: 399329 | Nonsense_ | 1101 | 375 | 34.06 | No | NA | NA | NA | NA |
| | | 97C > T | Mutation | | | | | | | | |
| BRCA1 | Sample1007 | g.chr17: 41243 | Missense_ | 875 | 235 | 26.86 | No | Damaging | Benign | driver | Neutral |
| | | 803T > G | Mutation | | | | | | | | |
| CDH1 | Sample1009 | g.chr16: 68857 | Missense_ | 465 | 16 | 3.44 | No | Tolerated | Benign | passenger | Neutral |
| | | 437C > T | Mutation | | | | | | | | |
| CHD4 | Sample1009 | g.chr12: 67016 | Missense_ | 562 | 193 | 34.34 | No | Tolerated | Possibly | driver | Neutral |
| | | 95C > T | Mutation | | | | | | damaging | | |
| CHD8 | Sample024 | g.chr14: 21871 | Missense_ | 1196 | 135 | 11.29 | No | Damaging | Possibly | driver | Deleterious |
| | | 630T > C | Mutation | | | | | | damaging | | |
| CHD8 | Sample1016 | g.chr14: 21883 | Missense_ | 309 | 131 | 42.39 | Yes | Damaging | Probably | driver | Deleterious |
| | | 749C > T | Mutation | | | | | | damaging | | |
| COL27A1 | Sample1003 | g.chr9: 117047 | Frameshift | 366 | 61 | 16.67 | No | NA | NA | NA | NA |
| | | 024delC | | | | | | | | | |
| CTCF | Sample1016 | g.chr16: 67654 | Missense_ | 731 | 300 | 41.04 | No | Damaging | Probably | passenger | Deleterious |
| | | 643G > A | Mutation | | | | | | damaging | | |
| DNAH11 | Sample1047* | g.chr7: 218562 | Missense | 682 | 217 | 31.82 | No | Tolerated | Benign | passenger | Deleterious |
| | | 18G > C | Mutation | | | | | | | | |
| DNAH11 | Sample1015 | g.chr7: 216308 | Missense_ | 300 | 69 | 23.00 | Yes | Damaging | Probably | passenger | Deleterious |
| | | 55C > T | Mutation | | | | | | damaging | | |
| DNAH11 | Sample1023 | g.chr7: 218263 | Missense_ | 732 | 159 | 21.72 | Yes | Damaging | Possibly | passenger | Deleterious |
| | | 51G > A | Mutation | | | | | | damaging | | |
| EGFR | Sample1047* | g.chr7: 552100 | Missense | 586 | 129 | 22.01 | Yes | Damaging | Possibly | driver | Neutral |
| | | 75T > G | Mutation | | | | | | damaging | | |
| EGFR | Sample1022 | g.chr7: 552100 | Missense_ | 817 | 220 | 26.93 | Yes | Damaging | Possibly | driver | Neutral |
| | | 75T > G | Mutation | | | | | | damaging | | |
| ERBB4 | Sample1014 | g.chr2: 212568 | Missense_ | 377 | 64 | 16.98 | No | Damaging | Probably | driver | Deleterious |
| | | 899A > G | Mutation | | | | | | damaging | | |
| ERBB4 | Sample1052 | g.chr2: 212248 | Nonsense_ | 398 | 197 | 49.50 | No | NA | NA | NA | NA |
| | | 765C > A | Mutation | | | | | | | | |
| FGFBP1 | Sample1003 | g.chr4: 159375 | Frameshift | 399 | 91 | 22.81 | No | NA | NA | NA | NA |
| | | 91delAG | | | | | | | | | |
| FLNA | Sample1005 | g.chrX: 153583 | Frameshift | 419 | 14 | 3.34 | No | NA | NA | NA | NA |
| | | 237delCACAC | | | | | | | | | |
| FLNA | Sample1037 | g.chrX: 153583 | Frameshift | 648 | 101 | 15.59 | No | NA | NA | NA | NA |
| | | 336delA | | | | | | | | | |
| FLNA | Sample1037 | g.chrX: 153583 | Frameshift | 634 | 72 | 11.36 | No | NA | NA | NA | NA |
| | | 339delCGTGCACACGGTGC | | | | | | | | | |
| FLNA | Sample1003 | g.chrX: 153583 | Inframe | 358 | 60 | 16.76 | No | NA | NA | NA | Deleterious |
| | | 283delTCGAAAGTGCCGTC | | | | | | | | | |
| | | CTCA | | | | | | | | | |
| FLNA | Sample1031 | g.chrX: 153588 | Inframe | 224 | 66 | 29.46 | No | NA | NA | NA | Deleterious |
| | | 907delGGG | | | | | | | | | |
| FLNA | Sample1038 | g.chrX: 153582 | Inframe | 386 | 14 | 3.63 | No | NA | NA | NA | Deleterious |
| | | 604delTTGTTGTCAG | | | | | | | | | |
| | | TG | | | | | | | | | |
| FLNA | Sample1039* | g.chrX: 153591 | Inframe | 549 | 95 | 17.30 | No | NA | NA | NA | Deleterious |
| | | 076delCCTTGAGCC | | | | | | | | | |
| FLNA | Sample1041* | g.chrX: 153586 | Inframe | 981 | 265 | 27.01 | No | NA | NA | NA | Deleterious |
| | | 688delCAT | | | | | | | | | |
| FLNA | Sample1056 | g.chrX: 153588 | Inframe | 587 | 145 | 24.70 | No | NA | NA | NA | Deleterious |
| | | 757InsGTTGTA | | | | | | | | | |
| FLNA | Sample1057 | g.chrX: 153583 | Inframe | 1091 | 287 | 26.31 | No | NA | NA | NA | Deleterious |
| | | 348delGGT | | | | | | | | | |
| FLNA | Sample1061* | g.chrX: 153583 | Inframe | 340 | 95 | 27.94 | No | NA | NA | NA | Deleterious |
| | | 241delCAG | | | | | | | | | |
| FLNA | Sample1048* | g.chrX: 153582 | Missense | 247 | 71 | 28.74 | No | Damaging | Probably | driver | Deleterious |
| | | 557T > A | Mutation | | | | | | damaging | | |
| FLNA | Sample1058* | g.chrX: 153588 | Missense | 343 | 111 | 32.36 | No | Damaging | Probably | passenger | Deleterious |
| | | 606G > C | Mutation | | | | | | damaging | | |
| FLNA | Sample1061* | g.chrX: 153594 | Missense | 195 | 76 | 38.97 | No | Tolerated | Benign | passenger | Neutral |
| | | 551T > C | Mutation | | | | | | | | |
| FLNA | Sample1064* | g.chrX: 153588 | Missense | 327 | 41 | 12.54 | No | Damaging | Probably | passenger | Deleterious |
| | | 567G > A | Mutation | | | | | | damaging | | |
| FLNA | Sample008 | g.chrX: 153588 | Missense_ | 441 | 110 | 24.94 | No | Damaging | Probably | passenger | Deleterious |
| | | 601C > T | Mutation | | | | | | damaging | | |
| FLNA | Sample1001 | g.chrX: 153588 | Missense_ | 348 | 90 | 25.86 | No | Damaging | Probably | passenger | Neutral |
| | | 616C > A | Mutation | | | | | | damaging | | |
| FLNA | Sample1002 | g.chrX: 153588 | Missense_ | 249 | 53 | 21.29 | No | Damaging | Possibly | passenger | Deleterious |
| | | 592C > T | Mutation | | | | | | damaging | | |
| FLNA | Sample1008 | g.chrX: 153588 | Missense_ | 297 | 35 | 11.78 | No | Damaging | Probably | passenger | Deleterious |
| | | 501T > G | Mutation | | | | | | damaging | | |
| FLNA | Sample1014 | g.chrX: 153588 | Missense_ | 210 | 33 | 15.71 | No | Damaging | Probably | passenger | Deleterious |
| | | 433G > A | Mutation | | | | | | damaging | | |
| FLNA | Sample1020 | g.chrX: 153588 | Missense_ | 269 | 113 | 42.01 | No | Damaging | Probably | driver | Deleterious |
| | | 460A > G | Mutation | | | | | | damaging | | |
| FLNA | Sample1023 | g.chrX: 153588 | Missense_ | 370 | 89 | 24.05 | No | Damaging | Probably | passenger | Deleterious |
| | | 567G > A | Mutation | | | | | | damaging | | |
| FLNA | Sample1024* | g.chrX: 153592 | Missense_ | 662 | 395 | 59.67 | No | Tolerated | Benign | passenger | Neutral |
| | | 736T > C | Mutation | | | | | | | | |
| FLNA | Sample1036 | g.chrX: 153588 | Missense_ | 538 | 94 | 17.47 | No | Damaging | Probably | passenger | Deleterious |
| | | 433G > A | Mutation | | | | | | damaging | | |
| FLNA | Sample1040* | g.chrX: 153588 | Missense_ | 916 | 242 | 26.42 | No | Damaging | Probably | passenger | Deleterious |
| | | 567G > A | Mutation | | | | | | damaging | | |
| FLNA | Sample1050 | g.chrX: 153588 | Missense_ | 178 | 64 | 35.96 | No | Tolerated | Probably | passenger | Deleterious |
| | | 456C > G | Mutation | | | | | | damaging | | |
| FLNA | Sample1051 | g.chrX: 153588 | Missense_ | 401 | 99 | 24.69 | No | Damaging | Possibly | passenger | Deleterious |
| | | 592C > T | Mutation | | | | | | damaging | | |
| FLNA | Sample1053 | g.chrX: 153595 | Missense_ | 427 | 158 | 37.00 | No | Damaging | Probably | driver | Deleterious |
| | | 842A > G | Mutation | | | | | | damaging | | |
| FLNA | Sample1076 | g.chrX: 153588 | Missense_ | 514 | 200 | 38.91 | No | Damaging | Probably | passenger | Deleterious |
| | | 720G > A | Mutation | | | | | | damaging | | |
| FLNA # | Sample1015 | g.chrX: 153586 | Missense_ | 130 | 30 | 23.08 | No | Damaging | Probably | driver | Deleterious |
| | | 590C > A | Mutation | | | | | | damaging | | |
| JAK2 | Sample1011 | g.chr9: 512678 | Missense_M | 516 | 193 | 37.40 | No | Damaging | Benign | passenger | Neutral |
| | | 7G > A | Mutation | | | | | | | | |
| MAP3K1 | Sample1004 | g.chr5: 561606 | Frameshift | 353 | 84 | 23.80 | No | NA | NA | NA | NA |
| | | 61InsG | | | | | | | | | |
| MAP3K1 | Sample1004 | g.chr5: 561617 | Frameshift | 531 | 91 | 17.14 | No | NA | NA | NA | NA |
| | | 30delA | | | | | | | | | |
| MAP3K1 | Sample1034 | g.chr5: 561687 | Frameshift | 799 | 42 | 5.26 | No | NA | NA | NA | NA |
| | | 49delA | | | | | | | | | |
| MAP3K1 | Sample1057 | g.chr5: 561769 | Frameshift | 18 | 3 | 16.67 | No | NA | NA | NA | NA |
| | | 50InsC | | | | | | | | | |
| MAP3K1 | Sample1023 | g.chr5: 561678 | Missense_ | 661 | 149 | 22.54 | No | Damaging | Probably | | |
| | | 23G > C | Mutation | | | | | | | | |
| MAP3K1 | Sample1027 | g.chrX: 703392 | Frameshift | 600 | 23 | 3.83 | No | NA | NA | NA | NA |
| | | 32delCGGCCTTG | | | | | | | | | |
| MED12 | Sample018 | g.chrX: 703392 | Inframe | 436 | 30 | 6.88 | No | NA | NA | NA | NA |
| | | 59delATAACCAGCCTGCTG | | | | | | | | | |
| | | TCTCTGGGGATG | | | | | | | | | |
| MED12 | Sample020 | g.chrX: 703386 | Inframe | 153 | 13 | 8.50 | No | NA | NA | NA | Deleterious |
| | | 80delCTCAGGACCCCAAAC | | | | | | | | | |
| | | AGAAGG | | | | | | | | | |
| MED12 | Sample023 | g.chrX: 703392 | inframe | 941 | 90 | 9.56 | No | NA | NA | NA | Deleterious |
| | | 37delTTGAATGTAAAACAA | | | | | | | | | |
| | | GGT | | | | | | | | | |
| MED12 | Sample024 | g.chrX: 703392 | inframe | 946 | 55 | 5.81 | No | NA | NA | NA | Deleterious |
| | | 42delTGTAAAACAAGGTTT | | | | | | | | | |
| | | CAATAACCAGCCTGC | | | | | | | | | |
| MED12 | Sample1002 | g.chrX: 703392 | Inframe | 271 | 9 | 3.32 | No | NA | NA | NA | Deleterious |
| | | 44delTAAAACAAGGTTTCA | | | | | | | | | |
| | | ATAACCAGC | | | | | | | | | |
| MED12 | Sample1015 | g.chrX: 703392 | Inframe | 241 | 33 | 13.52 | No | NA | NA | NA | Deleterious |
| | | 33delGGCCTTGAATGTAAA | | | | | | | | | |
| | | ACAAGGTTT | | | | | | | | | |
| MED12 | Sample1019 | g.chrX: 703392 | Inframe | 188 | 26 | 13.83 | No | NA | NA | NA | Deleterious |
| | | 40delAATGTAAAA | | | | | | | | | |
| MED12 | Sample1019 | g.chrX: 703392 | Inframe | 193 | 26 | 13.47 | No | NA | NA | NA | Deleterious |
| | | 51delAGGTTTCAATAACCA | | | | | | | | | |
| | | GCC | | | | | | | | | |
| MED12 | Sample1036 | g.chrX: 703392 | Inframe | 315 | 54 | 17.14 | No | NA | NA | NA | Deleterious |
| | | 37delTTGAATGTAAAACAA | | | | | | | | | |
| | | GGTTTCAATAACCAGCCTGC | | | | | | | | | |
| | | T | | | | | | | | | |
| MED12 | Sample1045 | g.chrX: 703392 | inframe | 971 | 83 | 8.55 | No | NA | NA | NA | Deleterious |
| | | 41delATGTAAAACAAGGTT | | | | | | | | | |
| | | TCAATAACC | | | | | | | | | |
| MED12 | Sample1046 | g.chrX: 703392 | inframe | 1067 | 60 | 5.62 | No | NA | NA | NA | Deleterious |
| | | 40delAATGTA | | | | | | | | | |
| MED12 | Sample1048* | g.chrX: 703392 | Inframe | 285 | 37 | 12.98 | No | NA | NA | NA | Deleterious |
| | | 45delAAAACAAGGTTTCAA | | | | | | | | | |
| | | TAACCAGCCTGC | | | | | | | | | |
| MED12 | Sample1050 | g.chrX: 703392 | Inframe | 319 | 119 | 37.30 | No | NA | NA | NA | Deleterious |
| | | 50delAAG | | | | | | | | | |
| MED12 | Sample1058* | g.chrX: 703392 | Inframe | 394 | 82 | 20.81 | No | NA | NA | NA | Deleterious |
| | | 45delAAAACAAGGTTTCAA | | | | | | | | | |
| MED12 | Sample1062* | g.chrX: 703392 | Inframe | 361 | 53 | 14.68 | No | NA | NA | NA | Deleterious |
| | | 57delCAATAACCAGCCTGC | | | | | | | | | |
| MED12 | Sample1072 | g.chrX: 703392 | Inframe | 516 | 107 | 20.74 | No | NA | NA | NA | Deleterious |
| | | 62delACCAGCCTGCTGTCT | | | | | | | | | |
| | | CTGGGGATG | | | | | | | | | |
| MED12 | Sample1074 | g.chrX: 703392 | Inframe | 393 | 61 | 15.52 | No | NA | NA | NA | Deleterious |
| | | 44delTAAAACAAGGT | | | | | | | | | |
| | | TTCAATAACCAGC | | | | | | | | | |
| MED12 | Sample1026 | g.chrX: 703392 | Splice_S ite | 545 | 77 | 14.13 | No | NA | NA | NA | |
| | | 21delGGATGAACTGAC | | | | | | | | | |
| MED12 | Sample001* | g.chrX: 703392 | Missense | 442 | 56 | 12.67 | Yes | Damaging | Probably | driver | Deleterious |
| | | 54G > A | Mutation | | | | | | damaging | | |
| MED12 | Sample013* | g.chrX: 703392 | Missense | 532 | 97 | 18.23 | Yes | Damaging | Probably | driver | Deleterious |
| | | 53G > C | Mutation | | | | | | damaging | | |
| MED12 | Sample014* | g.chrX: 703392 | Missense | 541 | 89 | 16.45 | Yes | Damaging | Probably | driver | Deleterious |
| | | 53G > A | Mutation | | | | | | damaging | | |
| MED12 | Sample016* | g.chrX: 703392 | Missense | 499 | 102 | 20.44 | Yes | Damaging | Probably | driver | Deleterious |
| | | 54G > A | Mutation | | | | | | damaging | | |
| MED12 | Sample1047* | g.chrX: 703392 | Missense | 463 | 131 | 28.29 | Yes | Damaging | Probably | driver | Deleterious |
| | | 53G > C | Mutation | | | | | | damaging | | |
| MED12 | Sample1059* | g.chrX: 703392 | Missense | 457 | 170 | 37.20 | Yes | Damaging | Probably | driver | Deleterious |
| | | 53G > T | Mutation | | | | | | damaging | | |
| MED12 | Sample1060* | g.chrX: 703392 | Missense | 358 | 87 | 24.30 | Yes | Damaging | Probably | driver | Deleterious |
| | | 53G > C | Mutation | | | | | | damaging | | |
| MED12 | Sample1063* | g.chrX: 703392 | Missense | 396 | 119 | 30.05 | Yes | Damaging | Probably | driver | Deleterious |
| | | 53G > A | Mutation | | | | | | damaging | | |
| MED12 | Sample1064* | g.chrX: 703392 | Missense | 336 | 111 | 33.04 | Yes | Damaging | Probably | driver | Deleterious |
| | | 54G > T | Mutation | | | | | | damaging | | |
| MED12 | Sample002 | g.chrX: 703392 | Missense_ | 458 | 59 | 12.88 | Yes | Damaging | Probably | driver | Deleterious |
| | | 54G > A | Mutation | | | | | | damaging | | |
| MED12 | Sample003 | g.chrX: 703392 | Missense_ | 480 | 72 | 15.00 | Yes | Damaging | Probably | driver | Deleterious |
| | | 54G > A | Mutation | | | | | | damaging | | |
| MED12 | Sample006 | g.chrX: 703392 | Missense_ | 91 | 15 | 16.48 | Yes | Damaging | Probably | driver | Deleterious |
| | | 54G > A | Mutation | | | | | | damaging | | |
| MED12 | Sample007 | g.chrX: 703392 | Missense_ | 595 | 194 | 32.61 | Yes | Damaging | Probably | driver | Deleterious |
| | | 54G > A | Mutation | | | | | | damaging | | |
| MED12 | Sample008 | g.chrX: 703392 | Missense_ | 241 | 63 | 26.14 | Yes | Damaging | Probably | driver | Deleterious |
| | | 54G > A | Mutation | | | | | | damaging | | |
| MED12 | Sample019 | g.chrX: 703392 | Missense_ | 489 | 130 | 26.58 | Yes | Damaging | Probably | driver | Deleterious |
| | | 54G > T | Mutation | | | | | | damaging | | |
| MED12 | Sample021 | g.chrX: 703392 | Missense_ | 1176 | 188 | 15.99 | Yes | Damaging | Probably | driver | Deleterious |
| | | 54G > A | Mutation | | | | | | damaging | | |
| MED12 | Sample022 | g.chrX: 703392 | Missense_ | 1075 | 58 | 5.40 | Yes | Damaging | Probably | driver | Deleterious |
| | | 54G > A | Mutation | | | | | | damaging | | |
| MED12 | Sample1003 | g.chrX: 703392 | Missense_ | 315 | 71 | 22.54 | Yes | Damaging | Probably | driver | Deleterious |
| | | 53G > T | Mutation | | | | | | damaging | | |
| MED12 | Sample1004 | g.chrX: 703392 | Missense_ | 329 | 48 | 14.59 | Yes | Damaging | Probably | driver | Deleterious |
| | | 53G > A | Mutation | | | | | | damaging | | |
| MED12 | Sample1005 | g.chrX: 703392 | Missense_ | 554 | 43 | 7.76 | Yes | Damaging | Probably | driver | Deleterious |
| | | 54G > T | Mutation | | | | | | damaging | | |
| MED12 | Sample1006 | g.chrX: 703392 | Missense_ | 440 | 82 | 18.64 | Yes | Damaging | Probably | driver | Deleterious |
| | | 53G > T | Mutation | | | | | | damaging | | |
| MED12 | Sample1007 | g.chrX: 703392 | Missense_ | 435 | 126 | 28.97 | Yes | Damaging | Probably | driver | Deleterious |
| | | 54G > T | Mutation | | | | | | damaging | | |
| MED12 | Sample1008 | g.chrX: 703392 | Missense_ | 502 | 96 | 19.12 | Yes | Damaging | Probably | driver | Deleterious |
| | | 51A > C | Mutation | | | | | | damaging | | |
| MED12 | Sample1010 | g.chrX: 703392 | Missense_ | 364 | 93 | 25.55 | No | Damaging | Probably | driver | Deleterious |
| | | 30T > G | Mutation | | | | | | damaging | | |
| MED12 | Sample1012 | g.chrX: 703392 | Missense_ | 254 | 50 | 19.69 | Yes | Damaging | Probably | driver | Deleterious |
| | | 54G > A | Mutation | | | | | | damaging | | |
| MED12 | Sample1016 | g.chrX: 703392 | Missense_ | 252 | 183 | 72.62 | yes | Damaging | Probably | driver | Deleterious |
| | | 54G > A | Mutation | | | | | | damaging | | |
| MED12 | Sample1020 | g.chrX: 703392 | Missense_ | 397 | 133 | 33.50 | Yes | Damaging | Probably | driver | Deleterious |
| | | 53G > A | Mutation | | | | | | damaging | | |
| MED12 | Sample1022 | g.chrX: 703392 | Missense_ | 562 | 169 | 30.07 | Yes | Damaging | Probably | driver | Deleterious |
| | | 54G > A | Mutation | | | | | | damaging | | |
| MED12 | Sample1023 | g.chrX: 703392 | Missense_ | 503 | 108 | 21.47 | Yes | Damaging | Probably | driver | Deleterious |
| | | 54G > A | Mutation | | | | | | damaging | | |
| MED12 | Sample1025* | g.chrX: 703392 | Missense_ | 1131 | 365 | 32.27 | Yes | Damaging | Probably | driver | Deleterious |
| | | 53G > A | Mutation | | | | | | damaging | | |
| MED12 | Sample1028 | g.chrX: 703392 | Missense_ | 724 | 209 | 28.87 | Yes | Damaging | Probably | driver | Deleterious |
| | | 54G > T | Mutation | | | | | | damaging | | |
| MED12 | Sample1029 | g.chrX: 703392 | Missense_ | 634 | 130 | 20.50 | Yes | Damaging | Probably | driver | Deleterious |
| | | 54G > A | Mutation | | | | | | damaging | | |
| MED12 | Sample1030 | g.chrX: 703392 | Missense_ | 570 | 120 | 21.05 | Yes | Damaging | Probably | driver | Deleterious |
| | | 54G > T | Mutation | | | | | | damaging | | |
| MED12 | Sample1031 | g.chrX: 703392 | Missense_ | 538 | 209 | 38.85 | Yes | Damaging | Probably | driver | Deleterious |
| | | 54G > A | Mutation | | | | | | damaging | | |
| MED12 | Sample1032 | g.chrX: 703392 | Missense_ | 641 | 62 | 9.67 | Yes | Damaging | Probably | driver | Deleterious |
| | | 53G > A | Mutation | | | | | | damaging | | |
| MED12 | Sample1034 | g.chrX: 703392 | Missense_ | 638 | 133 | 20.85 | Yes | Damaging | Probably | driver | Deleterious |
| | | 54G > A | Mutation | | | | | | damaging | | |
| MED12 | Sample1035 | g.chrX: 703392 | Missense_ | 559 | 140 | 25.04 | Yes | Damaging | Probably | passenger | Deleterious |
| | | 54G > C | Mutation | | | | | | damaging | | |
| MED12 | Sample1037 | g.chrX: 703392 | Missense_ | 552 | 184 | 33.33 | Yes | Damaging | Probably | driver | Deleterious |
| | | 54G > T | Mutation | | | | | | damaging | | |
| MED12 | Sample1038 | g.chrX: 703392 | Missense_ | 522 | 89 | 17.05 | Yes | Damaging | Probably | driver | Deleterious |
| | | 54G > A | Mutation | | | | | | damaging | | |
| MED12 | Sample1039* | g.chrX: 703392 | Missense_ | 1202 | 318 | 26.46 | Yes | Damaging | Probably | driver | Deleterious |
| | | 54G > T | Mutation | | | | | | damaging | | |
| MED12 | Sample1040* | g.chrX: 703392 | Missense_ | 1225 | 282 | 23.02 | Yes | Damaging | Probably | driver | Deleterious |
| | | 54G > T | Mutation | | | | | | damaging | | |
| MED12 | Sample1042* | g.chrX: 703392 | Missense_ | 1291 | 542 | 41.98 | Yes | Damaging | Probably | driver | Deleterious |
| | | 53G > A | Mutation | | | | | | damaging | | |
| MED12 | Sample1044* | g.chrX: 703392 | Missense_ | 1196 | 270 | 22.58 | Yes | Damaging | Probably | driver | Deleterious |
| | | 53G > A | Mutation | | | | | | damaging | | |
| MED12 | Sample1046 | g.chrX: 703392 | Missense_ | 1085 | 70 | 6.45 | No | Damaging | Probably | driver | Deleterious |
| | | 48A > C | Mutation | | | | | | damaging | | |
| MED12 | Sample1049 | g.chrX: 703392 | Missense_ | 349 | 13 | 3.72 | Yes | Damaging | Probably | driver | Deleterious |
| | | 54G > T | Mutation | | | | | | damaging | | |
| MED12 | Sample1051 | g.chrX: 703392 | Missense_ | 568 | 154 | 27.11 | Yes | Damaging | Probably | driver | Deleterious |
| | | 53G > A | Mutation | | | | | | damaging | | |
| MED12 | Sample1053 | g.chrX: 703392 | Missense_ | 548 | 184 | 33.58 | Yes | Damaging | Probably | passenger | Deleterious |
| | | 54G > C | Mutation | | | | | | damaging | | |
| MED12 | Sample1056 | g.chrX: 703392 | Missense_ | 591 | 239 | 40.44 | Yes | Damaging | Probably | driver | Deleterious |
| | | 54G > T | Mutation | | | | | | damaging | | |
| MED12 | Sample1065* | g.chrX: 703392 | Missense_ | 1460 | 518 | 35.48 | Yes | Damaging | Probably | driver | Deleterious |
| | | 53G > T | Mutation | | | | | | damaging | | |
| MED12 | Sample1067* | g.chrX: 703392 | Missense_ | 1248 | 422 | 33.81 | Yes | Damaging | Probably | driver | Deleterious |
| | | 53G > A | Mutation | | | | | | damaging | | |
| MED12 | Sample1069* | g.chrX: 703392 | Missense_ | 1214 | 333 | 27.43 | Yes | Damaging | Probably | driver | Deleterious |
| | | 54G > A | Mutation | | | | | | damaging | | |
| MED12 | Sample1071 | g.chrX: 703392 | Missense_ | 1148 | 418 | 36.41 | Yes | Damaging | Probably | driver | Deleterious |
| | | 54G > A | Mutation | | | | | | damaging | | |
| MED12 | Sample005 | g.chrX: 703392 | Splice_Site | 411 | 133 | 32.36 | Yes | Damaging | NA | passenger | Neutral |
| | | 15T > A | | | | | | | | | |
| MED12 | Sample1001 | g.chrX: 703392 | Splice_Site | 379 | 107 | 28.23 | Yes | Damaging | NA | passenger | Neutral |
| | | 15T > A | | | | | | | | | |
| MED12** | Sample1077* | g.chrX: 703392 | Missense_ | 1328 | 619 | 46.61 | Yes | Damaging | Probably | driver | Deleterious |
| | | 30T > G | Mutation | | | | | | damaging | | |
| MED12** | Sample1073* | g.chrX: 703392 | Missense | 249 | 121 | 48.59 | Yes | Damaging | Probably | driver | Deleterious |
| | | 54G > T | Mutation | | | | | | damaging | | |
| MLL2 | Sample013* | g.chr12: 49433 | Frameshift | 346 | 56 | 16.18 | No | NA | NA | NA | NA |
| | | 524delCT | | | | | | | | | |
| MLL2 | Sample1010 | g.chr12: 49435 | Frameshift | 208 | 31 | 14.90 | No | NA | NA | NA | NA |
| | | 198delG | | | | | | | | | |
| MLL2 | Sample1026 | g.chr12: 49415 | Frameshift | 859 | 221 | 25.73 | Yes | NA | NA | NA | NA |
| | | 900delCA | | | | | | | | | |
| MLL2 | Sample1043* | g.chr12: 49424 | Frameshift | 1092 | 205 | 18.77 | No | NA | NA | NA | NA |
| | | 805delG | | | | | | | | | |
| MLL2 | Sample1048* | g.chr12: 49446 | Frameshift | 183 | 45 | 24.59 | No | NA | NA | NA | NA |
| | | 165delG | | | | | | | | | |
| MLL2 | Sample1053 | g.chr12: 49443 | Frameshift | 521 | 148 | 28.41 | No | NA | NA | NA | NA |
| | | 860delG | | | | | | | | | |
| MLL2 | Sample1057 | g.chr12: 49433 | Frameshift | 564 | 118 | 20.92 | No | NA | NA | NA | NA |
| | | 545delCATGC | | | | | | | | | |
| MLL2 | Sample1071 | g.chr12: 49428 | frameshift | 988 | 218 | 22.06 | No | NA | NA | NA | NA |
| | | 434delAG | | | | | | | | | |
| MLL2 | Sample1076 | g.chr12: 49443 | Frameshift | 282 | 99 | 35.11 | No | NA | NA | NA | NA |
| | | 475delG | | | | | | | | | |
| MLL2 | Sample1070 | g.chr12: 49447 | Missense_ | 1462 | 300 | 20.52 | No | Damaging | Benign | passenger | Deleterious |
| | | 033T > A | Mutation | | | | | | | | |
| MLL2 | Sample1079 | g.chr12: 49428 | Missense_ | 528 | 26 | 4.92 | No | Damaging | Probably | passenger | Deleterious |
| | | 029G > C | Mutation | | | | | | damaging | | |
| MLL2 | Sample1019 | g.chr12: 49427 | Nonsense_ | 149 | 73 | 48.99 | No | NA | NA | NA | NA |
| | | 051G > A | Mutation | | | | | | | | |
| MLL2 | Sample1076 | g.chr12: 49443 | Nonsense_ | 304 | 120 | 39.47 | No | NA | NA | NA | NA |
| | | 956G > A | Mutation | | | | | | | | |
| NF1 | Sample1019 | g.chr17: 29684 | Frameshift | 668 | 591 | 88.47 | No | NA | NA | NA | NA |
| | | 021delAA | | | | | | | | | |
| NF1 | Sample1050 | g.chr17: 29490 | Frameshift | 340 | 87 | 25.59 | No | NA | NA | NA | NA |
| | | 332delG | | | | | | | | | |
| NF1 | Sample1050 | g.chr17: 29665 | Frameshift | 345 | 100 | 28.99 | No | NA | NA | NA | NA |
| | | 751delACTT | | | | | | | | | |
| NF1 | Sample1021 | g.chr17: 29559 | Missense_ | 332 | 16 | 4.82 | No | Tolerated | Possibly | driver | Deleterious |
| | | 719T > C | Mutation | | | | | | damaging | | |
| NF1 | Sample1022 | g.chr17: 29528 | Nonsense_ | 427 | 16 | 3.75 | Yes | NA | NA | NA | NA |
| | | 489C > T | Mutation | | | | | | | | |
| NF1 | Sample1050 | g.chr17: 29557 | Nonsense_ | 347 | 12 | 3.46 | No | NA | NA | NA | NA |
| | | 327A > T | Mutation | | | | | | | | |
| NF1 | Sample1076 | g.chr17: 29527 | Nonsense_ | 327 | 227 | 69.42 | No | NA | NA | NA | NA |
| | | 503G > T | Mutation | | | | | | | | |
| PCLO | Sample010 | g.chr7: 825806 | Missense_ | 495 | 133 | 26.87 | No | Damaging | Benign | passenger | Deleterious |
| | | 90G > A | Mutation | | | | | | | | |
| PCLO | Sample1002 | g.chr7: 825813 | Missense_ | 567 | 120 | 21.16 | No | Damaging | Probably | passenger | Deleterious |
| | | 06C > A | Mutation | | | | | | damaging | | |
| PCLO | Sample1013 | g.chr7: 827639 | Missense_ | 543 | 38 | 7.00 | No | Tolerated | Benign | passenger | Neutral |
| | | 70G > T | Mutation | | | | | | | | |
| PCNXIA | Sample002 | g.chr14: 60582 | Frameshift | 689 | 37 | 5.37 | No | NA | NA | NA | NA |
| | | 052delGT | | | | | | | | | |
| PCNXIA | Sample1011 | g.chr14: 60582 | Frameshift | 570 | 235 | 41.23 | No | NA | NA | NA | NA |
| | | 497delAG | | | | | | | | | |
| PIK3CA | Sample1017 | g.chr3: 178927 | Frameshift | 518 | 220 | 42.47 | No | NA | NA | NA | NA |
| | | 976delC | | | | | | | | | |
| PIK3CA | Sample1017 | g.chr3: 178927 | Frameshift | 501 | 221 | 44.11 | No | NA | NA | NA | NA |
| | | 978delCTGTC | | | | | | | | | |
| PIK3CA | Sample1012 | g.chr3: 178927 | Inframe | 382 | 63 | 16.49 | No | NA | NA | NA | Deleterious |
| | | 983delCAT | | | | | | | | | |
| PIK3CA | Sample1047* | g.chr3: 178917 | Missense | 541 | 81 | 14.97 | No | Damaging | Benign | passenger | Neutral |
| | | 550A > C | Mutation | | | | | | | | |
| PIK3CA | Sample1011 | g.chr3: 178952 | Missense_ | 642 | 280 | 43.61 | Yes | Tolerated | Benign | driver | Neutral |
| | | 085A > T | Mutation | | | | | | | | |
| PIK3CA | Sample1012 | g.chr3: 178927 | Missense_ | 382 | 63 | 16.49 | No | Damaging | Probably | passenger | Deleterious |
| | | 987T > G | Mutation | | | | | | damaging | | |
| PIK3CA | Sample1068* | g.chr3: 178952 | Missense_ | 1772 | 549 | 30.98 | Yes | Damaging | Benign | driver | Neutral |
| | | 085A > G | Mutation | | | | | | | | |
| PIK3CG | Sample1022 | g.chr7: 106509 | Missense_ | 790 | 177 | 22.41 | No | Damaging | Benign | passenger | Neutral |
| | | 548G > A | Mutation | | | | | | | | |
| PTEN | Sample1074 | g.chr10: 89653 | Inframe | 465 | 339 | 72.90 | No | NA | NA | NA | Deleterious |
| | | 795delATT | | | | | | | | | |
| RARA | Sample019 | g.chr17: 38512 | Frameshift | 96 | 5 | 5.21 | No | NA | NA | NA | NA |
| | | 375delT | | | | | | | | | |
| RARA | Sample1001 | g.chr17: 38512 | Frameshift | 69 | 25 | 36.23 | No | NA | NA | NA | NA |
| | | 306delGCCGCCTCTC | | | | | | | | | |
| RARA | Sample1003 | g.chr17: 38512 | Frameshift | 84 | 19 | 22.62 | No | NA | NA | NA | NA |
| | | 311delCT | | | | | | | | | |
| RARA | Sample1061* | g.chr17: 38512 | Frameshift | 84 | 30 | 35.71 | No | NA | NA | NA | |
| | | 259delG | | | | | | | | | |
| RARA | Sample1012 | g.chr17: 38510 | Inframe | 143 | 38 | 26.57 | No | NA | NA | NA | Deleterious |
| | | 600delCTT | | | | | | | | | |
| RARA | Sample1043* | g.chr17: 38510 | Inframe | 770 | 168 | 21.82 | No | NA | NA | NA | Deleterious |
| | | 600delCTT | | | | | | | | | |
| RARA | Sample1046 | g.chr17: 38512 | inframe | 282 | 30 | 10.64 | No | NA | NA | NA | Deleterious |
| | | 314delTCA | | | | | | | | | |
| RARA | Sample1072 | g.chr17: 38510 | Inframe | 363 | 107 | 29.48 | No | NA | NA | NA | Deleterious |
| | | 600delCTT | | | | | | | | | |
| RARA | Sample014* | g.chr17: 38510 | Missense | 318 | 71 | 22.33 | No | Damaging | Probably | passenger | Deleterious |
| | | 606C > T | Mutation | | | | | | damaging | | |
| RARA | Sample1058* | g.chr17: 38510 | Missense | 284 | 92 | 32.39 | No | Damaging | Probably | passenger | Deleterious |
| | | 611G > C | Mutation | | | | | | damaging | | |
| RARA | Sample1062* | g.chr17: 38512 | Missense | 92 | 27 | 29.35 | No | Damaging | Probably | passenger | Deleterious |
| | | 270G > A | Mutation | | | | | | damaging | | |
| RARA | Sample1063* | g.chr17: 38510 | Missense | 244 | 54 | 22.13 | No | Damaging | Probably | passenger | Deleterious |
| | | 641A > C | Mutation | | | | | | damaging | | |
| RARA | Sample1064* | g.chr17: 38512 | Missense | 114 | 35 | 30.70 | No | Damaging | Probably | driver | Deleterious |
| | | 315T > G | Mutation | | | | | | damaging | | |
| RARA | Sample007 | g.chr17: 38510 | Missense_ | 390 | 104 | 26.67 | Yes | Damaging | Possibly | passenger | Deleterious |
| | | 626C > T | Mutation | | | | | | damaging | | |
| RARA | Sample1007 | g.chr17: 38510 | Missense_ | 357 | 86 | 24.09 | No | Tolerated | Possibly | driver | Deleterious |
| | | 642A > G | Mutation | | | | | | damaging | | |
| RARA | Sample1015 | g.chr17: 38510 | Missense_ | 239 | 81 | 33.89 | No | Damaging | Probably | passenger | Deleterious |
| | | 611G > A | Mutation | | | | | | damaging | | |
| RARA | Sample1022 | g.chr17: 38510 | Missense_ | 163 | 47 | 28.83 | No | Damaging | Probably | passenger | Deleterious |
| | | 560C > G | Mutation | | | | | | damaging | | |
| RARA | Sample1023 | g.chr17: 38510 | Missense_ | 292 | 55 | 18.84 | Yes | Damaging | Probably | driver | Deleterious |
| | | 603T > C | Mutation | | | | | | damaging | | |
| RARA | Sample1027 | g.chr17: 38510 | Missense_ | 311 | 52 | 16.72 | No | Damaging | Probably | passenger | Deleterious |
| | | 606C > T | Mutation | | | | | | damaging | | |
| RARA | Sample1028 | g.chr17: 38512 | Missense_ | 176 | 56 | 31.82 | No | Damaging | Probably | passenger | Deleterious |
| | | 270G > A | Mutation | | | | | | damaging | | |
| RARA | Sample1034 | g.chr17: 38512 | Missense_ | 104 | 21 | 20.19 | No | Damaging | Probably | passenger | Deleterious |
| | | 270G > A | Mutation | | | | | | damaging | | |
| RARA | Sample1035 | g.chr17: 38510 | Missense_ | 398 | 130 | 32.66 | No | Damaging | Probably | passenger | Deleterious |
| | | 606C > T | Mutation | | | | | | damaging | | |
| RARA | Sample1036 | g.chr17: 38512 | Missense_ | 488 | 48 | 9.84 | No | Damaging | Probably | passenger | Deleterious |
| | | 308C > T | Mutation | | | | | | damaging | | |
| RARA | Sample1037 | g.chr17: 38512 | Missense_ | 556 | 43 | 7.73 | No | Damaging | Probably | driver | Deleterious |
| | | 315T > G | Mutation | | | | | | damaging | | |
| RARA | Sample1042* | g.chr17: 38512 | Missense_ | 291 | 116 | 39.86 | No | Damaging | Probably | driver | Deleterious |
| | | 270G > T | Mutation | | | | | | damaging | | |
| RARA | Sample1045 | g.chr17: 38510 | Missense_ | 710 | 178 | 25.07 | No | Tolerated | Possibly | passenger | Deleterious |
| | | 596A > G | Mutation | | | | | | damaging | | |
| RARA | Sample1056 | g.chr17: 38510 | Missense_ | 395 | 159 | 40.25 | No | Damaging | Probably | passenger | Deleterious |
| | | 606C > T | Mutation | | | | | | damaging | | |
| RARA | Sample1067* | g.chr17: 38510 | Missense_ | 936 | 264 | 28.21 | No | Damaging | Probably | passenger | Deleterious |
| | | 641A > C | Mutation | | | | | | damaging | | |
| RARA | Sample1068* | g.chr17: 38512 | Missense_ | 269 | 80 | 29.74 | No | Damaging | Probably | passenger | Deleterious |
| | | 309C > A | Mutation | | | | | | damaging | | |
| RARA | Sample1069* | g.chr17: 38510 | Missense_ | 978 | 328 | 33.54 | No | Damaging | Probably | passenger | Deleterious |
| | | 645C > A | Mutation | | | | | | damaging | | |
| RARA | Sample1071 | g.chr17: 38508 | Missense_ | 1037 | 372 | 35.87 | No | Tolerated | Probably | passenger | Neutral |
| | | 622C > T | utation | | | | | | damaging | | |
| RARA | Sample1004 | g.chr17: 38512 | 40 | 8 | 20.00 | No | NA | NA | NA | NA | Splice_Site |
| | | 256T > A | | | | | | | | | |
| RB1 | Sample1021 | g.chr13: 49030 | Frameshift | 571 | 40 | 7.01 | No | NA | NA | NA | NA |
| | | 472delT | | | | | | | | | |
| RB1 | Sample1021 | g.chr13: 49039 | Frameshift | 955 | 64 | 6.70 | No | NA | NA | NA | NA |
| | | 403InsTT | | | | | | | | | |
| RB1 | Sample1067* | g.chr13: 49030 | Frameshift | 1231 | 133 | 10.80 | No | NA | NA | NA | NA |
| | | 408delAG | | | | | | | | | |
| RB1 | Sample1067* | g.chr13: 48941 | Frameshift | 564 | 222 | 39.36 | No | NA | NA | NA | NA |
| | | 637delTCTT | | | | | | | | | |
| RB1 | Sample1060* | g.chr13: 48936 | Missense | 435 | 195 | 44.83 | No | Damaging | Benign | passenger | Neutral |
| | | 980C > T | Mutation | | | | | | | | |
| RB1 | Sample1023 | g.chr13: 48937 | Missense_ | 541 | 123 | 22.74 | No | Tolerated | Benign | passenger | Neutral |
| | | 022G > A | Mutation | | | | | | | | |
| RB1 | Sample1019 | g.chr13: 48955 | Nonsense_ | 256 | 222 | 86.72 | No | NA | NA | NA | NA |
| | | 394C > T | Mutation | | | | | | | | |
| RBM6 | Sample1011 | g.chr3: 500954 | Missense_ | 152 | 100 | 65.79 | No | Tolerated | Possibly | passenger | Neutral |
| | | 15C > T | Mutation | | | | | | damaging | | |
| ROS1 | Sample010 | g.chr6: 117718 | Missense_ | 72 | 28 | 38.89 | No | Damaging | Possibly | passenger | Neutral |
| | | 103A > T | Mutation | | | | | | damaging | | |
| ROS1 | Sample1077* | g.chr6: 117710 | Missense_ | 912 | 832 | 92.23 | No | Damaging | Possibly | passenger | Neutral |
| | | 680C > A | Mutation | | | | | | damaging | | |
| RUNX1 | Sample1046 | g.chr21: 36164 | Frameshift | 683 | 36 | 5.27 | No | NA | NA | NA | NA |
| | | 851insG | | | | | | | | | |
| RUNX1 | Sample1017 | g.chr21: 36252 | Missense_ | 721 | 23 | 3.19 | No | Damaging | Possibly | driver | Deleterious |
| | | 925T > G | Mutation | | | | | | damaging | | |
| SETD2 | Sample1002 | g.chr3: 471394 | Frameshift | 531 | 114 | 21.47 | No | NA | NA | NA | NA |
| | | 57delC | | | | | | | | | |
| SETD2 | Sample1010 | g.chr3: 471630 | Frameshift | 535 | 28.97 | No | NA | NA | NA | NA | NA |
| | | 91delT | | | | | | | | | |
| SETD2 | Sample1011 | g.chr3: 471395 | Frameshift | 442 | 291 | 65.84 | No | NA | NA | NA | NA |
| | | 03delCT | | | | | | | | | |
| SETD2 | Sample1025* | g.chr: 471430 | Frameshift | 860 | 507 | 58.95 | No | NA | NA | NA | NA |
| | | 09insA | | | | | | | | | |
| SETD2 | Sample1026 | g.chr3: 471296 | Frameshift | 895 | 50 | 5.59 | No | NA | NA | NA | NA |
| | | 89delAG | | | | | | | | | |
| SETD2 | Sample1027 | g.chr3: 471252 | Frameshift | 970 | 130 | 13.40 | No | NA | NA | NA | NA |
| | | 63delTG | | | | | | | | | |
| SETD2 | Sample1036 | g.chr3: 471626 | Frameshift | 632 | 179 | 28.32 | No | NA | NA | NA | NA |
| | | 66delTATT | | | | | | | | | |
| SETD2 | Sample1043* | g.chr3: 471649 | Frameshift | 1536 | 276 | 17.97 | No | NA | NA | NA | NA |
| | | 70delTCTT | | | | | | | | | |
| SETD2 | Sample1047* | g.chr3: 470612 | Frameshift | 588 | 198 | 33.67 | No | NA | NA | NA | NA |
| | | 67delT | | | | | | | | | |
| SETD2 | Sample1048* | g.chr3: 471037 | Frameshift | 451 | 109 | 24.17 | No | NA | NA | NA | NA |
| | | 29delTTTAT | | | | | | | | | |
| SETD2 | Sample1063* | g.chr3: 471475 | Frameshift | 530 | 299 | 56.42 | No | NA | NA | NA | NA |
| | | 64delTT | | | | | | | | | |
| SETD2 | Sample1065* | g.chr3: 470840 | Frameshift | 1621 | 585 | 36.09 | No | NA | NA | NA | NA |
| | | 93delG | | | | | | | | | |
| SETD2 | Sample1069* | g.chr3: 471296 | Frameshift | 1324 | 402 | 30.36 | No | NA | NA | NA | NA |
| | | 16delG | | | | | | | | | |
| SETD2 | Sample1077* | g.chr3: 471618 | Frameshift | 1942 | 1612 | 83.01 | No | NA | NA | NA | NA |
| | | 87delCTCT | | | | | | | | | |
| SETD2 | Sample1001 | g.chr3: 471395 | Inframe | 483 | 68 | 14.08 | No | NA | NA | NA | Deleterious |
| | | 63delCTT | | | | | | | | | |
| SETD2 | Sample1016 | g.chr3: 471258 | Inframe | 280 | 170 | 60.71 | No | NA | NA | NA | Deleterious |
| | | 40delGGAATGGGCAA | | | | | | | | | |
| | | G | | | | | | | | | |
| SETD2 | Sample1048* | g.chr3: 471394 | Inframe | 508 | 122 | 24.02 | No | NA | NA | NA | Deleterious |
| | | 50delCTT | | | | | | | | | |
| SETD2 | Sample1060* | g.chr3: 471395 | Inframe | 414 | 104 | 25.12 | No | NA | NA | NA | Deleterious |
| | | 63delCTT | | | | | | | | | |
| SETD2 | Sample1047* | g.chr3: 470591 | Missense | 424 | 138 | 32.55 | No | Damaging | Probably | driver | Deleterious |
| | | 43T > G | Mutation | | | | | | damaging | | |
| SETD2 | Sample1060* | g.chr3: 471475 | Missense | 536 | 97 | 18.10 | No | Damaging | Probably | driver | Deleterious |
| | | 12T > C | Mutation | | | | | | damaging | | |
| SETD2 | Sample1015 | g.chr3: 471448 | Missense_ | 405 | 113 | 27.90 | No | Damaging | Probably | driver | Deleterious |
| | | 76A > C | Mutation | | | | | | damaging | | |
| SETD2 | Sample1027 | g.chr3: 470591 | Missense_ | 525 | 26 | 4.95 | No | Damaging | Probably | driver | Deleterious |
| | | 33G > A | Mutation | | | | | | damaging | | |
| SETD2 | Sample1031 | g.chr3: 471395 | Missense_ | 774 | 269 | 34.75 | No | Damaging | Probably | driver | Deleterious |
| | | 54C > T | Mutation | | | | | | damaging | | |
| SETD2 | Sample1039* | g.chr3: 471553 | Missense_ | 1598 | 406 | 25.41 | No | Damaging | Probably | driver | Deleterious |
| | | 99C > A | Mutation | | | | | | damaging | | |
| SETD2 | Sample1010 | g.chr3: 471554 | Nonsense_ | 522 | 132 | 25.29 | No | NA | NA | NA | NA |
| | | 48G > A | Mutation | | | | | | | | |
| SETD2 | Sample1023 | g.chr3: 471037 | Nonsense_ | 900 | 197 | 21.89 | No | NA | NA | NA | NA |
| | | 59T > A | Mutation | | | | | | | | |
| SETD2 | Sample1069* | g.chr3: 471642 | Nonsense_ | 1267 | 388 | 30.62 | Yes | NA | NA | NA | NA |
| | | 68G > A | Mutation | | | | | | | | |
| SF3B1 | Sample1016 | g.chr2: 198267 | Missense_ | 648 | 279 | 43.06 | No | Damaging | Probably | passenger | Deleterious |
| | | 720A > C | Mutation | | | | | | damaging | | |
| SMAD4 | Sample1010 | g.chr18: 48573 | Missense_ | 424 | 90 | 21.23 | No | Tolerated | Probably | passenger | Neutral |
| | | 563G > C | Mutation | | | | | | damaging | | |
| STAT3 | Sample1011 | g.chr17: 40481 | Frameshift | 369 | 125 | 33.88 | No | NA | NA | NA | NA |
| | | 765delCT | | | | | | | | | |
| SYNE1 | Sample1064* | g.chr6: 152623 | Missense | 229 | 102 | 44.54 | No | Damaging | Possibly | passenger | Neutral |
| | | 012C > T | Mutation | | | | | | damaging | | |
| SYNE1 | Sample1004 | g.chr6: 152774 | Missense_ | 463 | 70 | 15.12 | Yes | Damaging | Probably | driver | Deleterious |
| | | 743C > T | Mutation | | | | | | damaging | | |
| TBX3 | Sample024 | g.chr12: 11511 | Frameshift | 1371 | 134 | 9.77 | No | NA | NA | NA | NA |
| | | 7310delT | | | | | | | | | |
| TET3 | Sample1013 | g.chr2: 742735 | Missense_ | 245 | 92 | 37.55 | No | Damaging | Possibly | passenger | Neutral |
| | | 47T > C | Mutation | | | | | | damaging | | |
| TP53 | Sample1019 | g.chr17: 75770 | Frameshift | 417 | 368 | 88.25 | No | NA | NA | NA | NA |
| | | 57delCC | | | | | | | | | |
| TP53 | Sample1021 | g.chr17: 75775 | Frameshift | 327 | 30 | 9.17 | No | NA | NA | NA | NA |
| | | 93delAC | | | | | | | | | |
| TP53 | Sample1073* | g.chr17: 75771 | Missense | 205 | 126 | 61.46 | Yes | Damaging | Probably | driver | Deleterious |
| | | 29A > G | Mutation | | | | | | damaging | | |
| ZBED4 | Sample1002 | g.chr22: 50279 | Missense_ | 228 | 48 | 21.05 | No | Tolerated | Benign | passenger | Neutral |
| | | 904A > G | Mutation | | | | | | | | |

Note:
Recurrent genes are in bold
*Samples with no matched normal tissue
‡COSMIC version v69 was used
***CHASM pvalue <0.05(driver) > 0.05(passenger)
#Detected in exome sequencing and validated by Sanger sequencing for cDNA + gDNA. Variant reads present in targeted sequencing but not called due to strand bias.
**Filtered out due to allele frequency >45% and <55% in tumor-only sample, but retained as this mutation has been confirmed to be somatic in other paried samples
(and also listed in COSMIC)

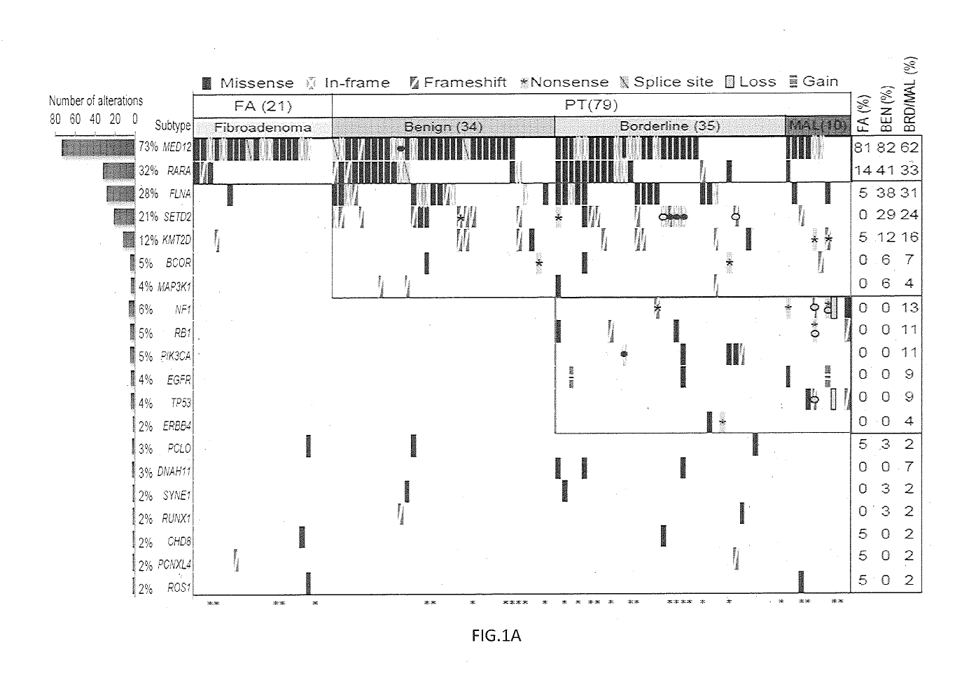

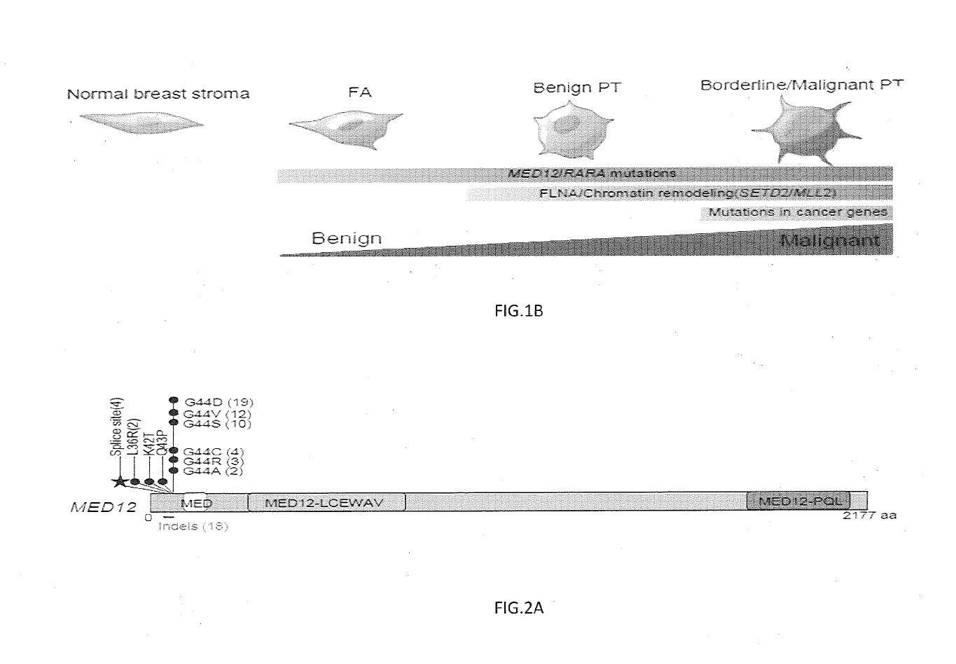

From the experiments carried as described above, the present disclosure identified 20 recurrently mutated genes in the fibroepithelial tumors as being summarized in FIG. 1a and Table 4. In addition, the present disclosure used OncoCNV²⁸ to detect copy number alterations in targeted genes. The acquired results as revealed in FIG. 1A, FIG. 3D and Table 6 confirmed loss-of-heterozygosity (LOH) patterns for mutations in samples with exome sequencing data using Control-FREEC²⁹.

TABLE 5
Copy number alterations of the 50 targeted genes in 100 fibroepithelial tumors

| Specimen | Gene | Transcript | | | | Copy | | |
|---|---|---|---|---|---|---|---|---|
| ID | Symbol | ID | Chromosome | Start | End | Number | P-value | Q-value |
| Sample1076 | EGFR | CCDS5514.1 | chr7 | 55086911 | 55273341 | 18 | 1.56E−15 | 3.27E−14 |
| Sample1056 | EGFR | CCDS5514.1 | chr7 | 55086911 | 55273341 | 11.5 | 2.23E−15 | 4.24E−14 |
| Sample1076 | NF1 | CCDS42292.1 | chr17 | 29422297 | 29701221 | 1 | 9.78E−34 | 2.25E−32 |
| Sample1078 | NF1 | CCDS42292.1 | chr17 | 29422297 | 29592421 | 1 | 1.88E−18 | 4.52E−17 |
| Sample1078 | NF1 | CCDS42292.1 | chr17 | 29652813 | 29665892 | 0 | 1.89E−08 | 4.16E−07 |
| Sample1074 | PTEN | CCDS31238.1 | chr10 | 89624175 | 89725256 | 1 | 2.84E−05 | 5.10E−04 |
| Sample1011 | SETD2 | CCDS2749.2 | chr3 | 47058543 | 47205468 | 1 | 6.35E−17 | 1.27E−15 |
| Sample1078 | TP53 | CCDS45606.1 | chr17 | 7569531 | 7579965 | 1 | 4.82E−07 | 9.64E−06 |

Note:
Genes with copy number estimates less than 1.5 or more than 10 were considered to have copy number losses or gains. For copy number alterations, only loss of tumor supressor genes and amplification of oncogenes are included. P-values and q-values are generated by the OncoCNV algorithm.

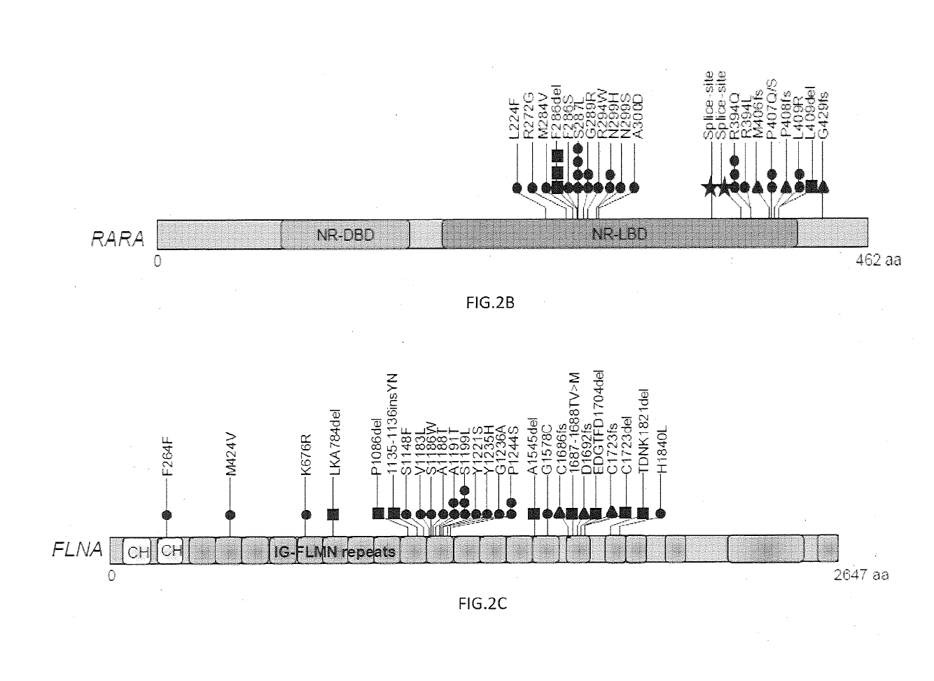

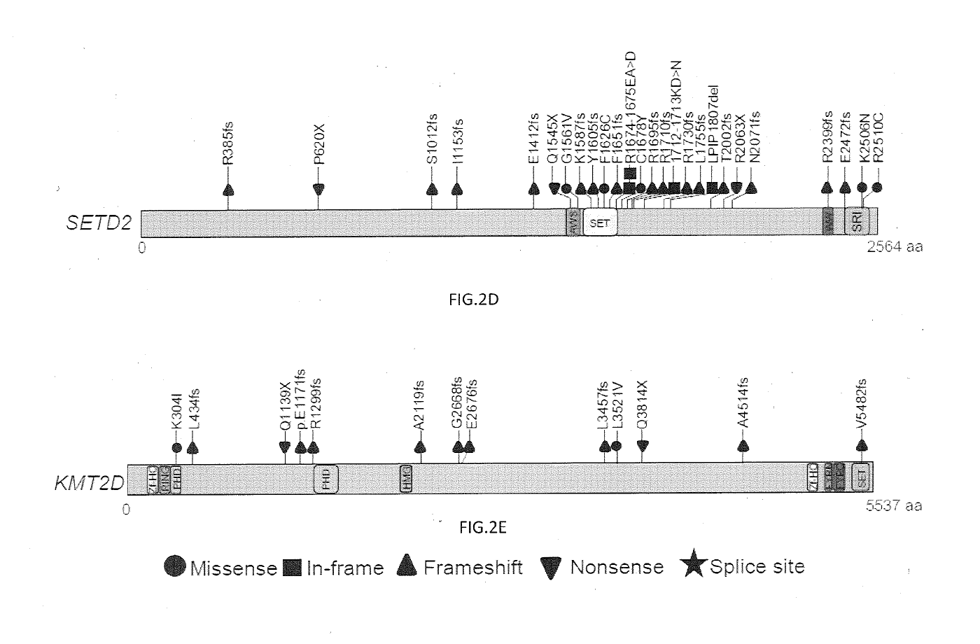

A comparison of recurrent mutations across the fibroepithelial tumors revealed distinct patterns of mutations and pathways associated with different phases in the breast fibroepithelial tumor spectrum as shown in FIGS. 1A and 1B. First, mutations in MED12 and RARA (nuclear retinoic acid receptor alpha), were frequently found in all phases of fibroepithelial tumors, occurring in 73% and 32% of tumors respectively. Confirming earlier studies, the MED12 exon 2 mutations in fibroepithelial tumors were identical to those reported in uterine leiomyomas, but were distinct in both pattern and location from MED12 mutations found in prostate and adrenocortical carcinoma^{30,31}. Notably, the present disclosure also observed RARA mutations in more than one-third of the fibroepithelial tumors (FIG. 2B). Prior to the present study, somatic mis sense mutations in PML-RARA have only been previously reported in therapy-resistant acute promyelocytic leukemia (APL)³², or sporadically in other solid tumors at low frequencies (<5%). The fibroepithelial RARA mutations were highly clustered within the nuclear hormone receptor ligand binding domain (LBD) and comprised missense mutations and in-frame deletions, consistent with these mutations possibly affecting interactions between RARA and other binding partners. Interestingly, MED12 and RARA have both been associated with estrogen signalling and estrogen-regulated transcription^{33,34}, and mutations in MED12 and RARA co-occurred at rates higher than expected by chance (permutation test p-value=0.0046, 100000 trials). These results suggest that PTs and FAs may share a common origin, where MED12 and RARA mutations are early events which may interact or collaborate to cause hormonal dysregulation in this tumor type.

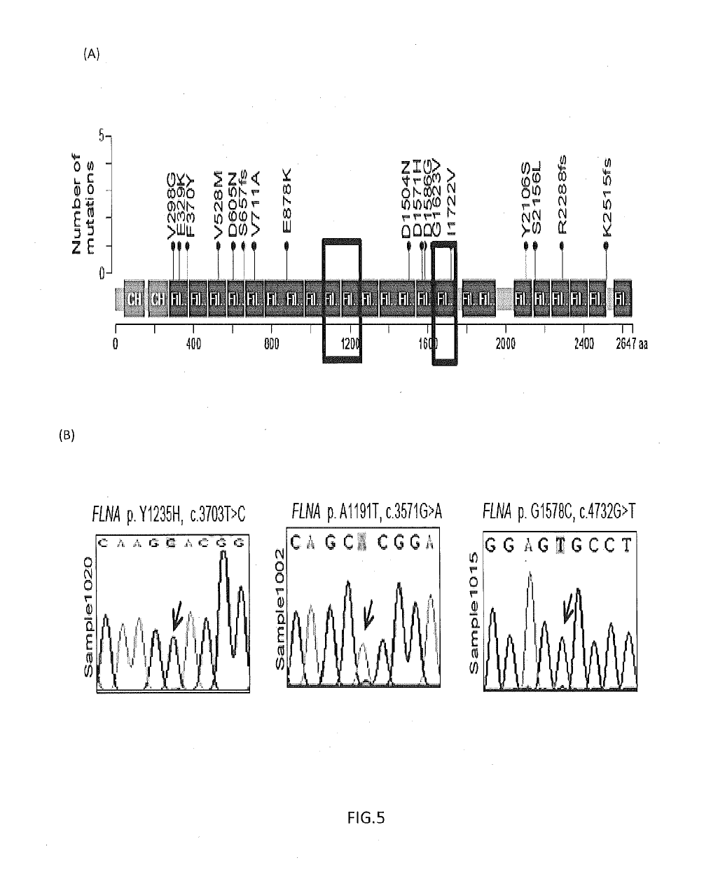

Second, the present disclosure also observed novel mutations in FLNA, SETD2, MLL2, BCOR and MAP3K1 in PTs (benign, borderline and malignant) that were rarely present in FAs (FIG. 1A, Fisher's exact test p-value compared to FA=1E-04). This finding suggests that PT tumorigenesis is likely to involve these additional mutated genes. Of these, FLNA is particularly novel. An X chromosome gene, FLNA encodes filamin A, a F-actin cross-linking protein, which functions as a scaffolding protein regulating signalling events involved in cell motility and invasion by interacting with integrin receptors³⁵. The FLNA mutations in fibroepithelial tumors (28%, 28/100) were specifically observed in the F-actin binding regions, particularly in immunoglobulin (Ig)-like repeat 9-15 domains (80%, 24/30)³⁶. In contrast, FLNA mutations in BCs, while reported, have been mainly found to affect other Ig-like repeat domains rather than the F-actin binding region as illustrated in FIG. 5A and FIG. 5B. These results suggest a functional role for FLNA in PT pathogenesis that may be distinct to that in BCs. Using cDNA Sanger sequencing, the present disclosure confirmed expression of the FLNA mutant transcripts, indicating that the FLNA mutations are likely to occur on the active X chromosome.

Besides FLNA, over one third (35%) of PTs also harboured mutations in at least one of two chromatin modifying enzymes; SETD2 (21%) and MLL2 (12%) (Fisher's test p-value compared to FA=0.0058 and further in view of FIG. 1A). The SETD2 and MLL2 mutations showed a classical tumor suppressor loss-of-function mutation pattern comprising inactivating mutations and deletions^{37, 38}. Both SETD2 and MLL2 are histone methyltransferases that mediate epigenetic regulation and the inactivation of these genes may result in aberrant transcriptional regulation through chromatin modification.

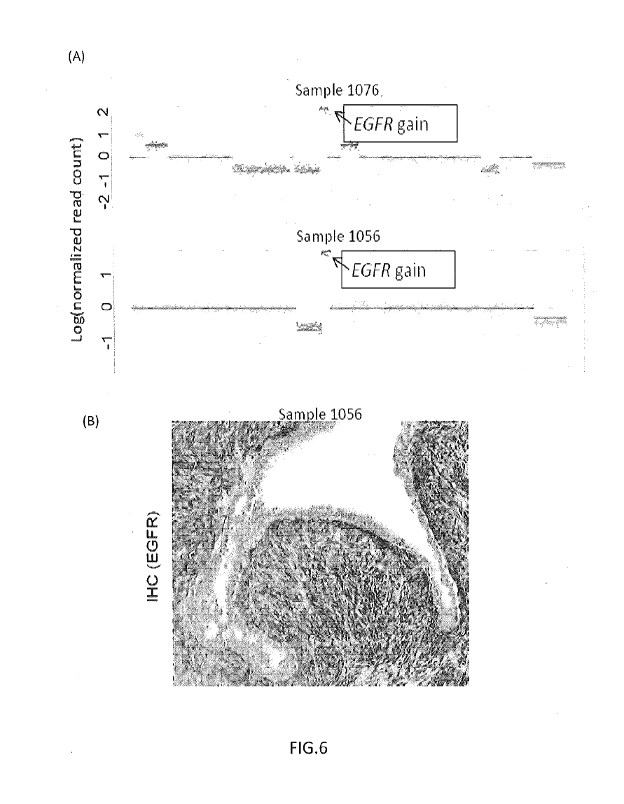

Third, compared to benign PTs, borderline and malignant PTs also exhibited additional mutations in NF1, RB1, TP53, PIK3CA, ERBB4 and EGFR, which are known cancer-driver genes that have transforming ability. Copy number alterations (CNAs) of these genes were also found in borderline/malignant PTs. These findings are consistent with previous studies whereby TP53 and RB1 were found to be deregulated in malignant PTs³⁹⁻⁴¹. Interestingly, although the frequency of alterations in each individual cancer-related gene was low, 29% (13/45) of borderline/malignant PTs exhibited probable driver alterations (defined as either COSMIC recurrent mutations and loss-of-function mutations (nonsense/frameshift) or high-level CNAs) in at least one cancer-related gene. In contrast, none of the 55 FAs and benign PTs (0/55, 0%) harboured genetic alterations in these genes (Fisher's exact test, p-value=1.02E-05). These results suggest that these cancer-related genes may be involved in a subset of higher-grade PTs. Notably, two tumors clearly contained bona-fide PIK3CA activating mutations (H1047R/L), and two tumors harboured high level EGFR amplifications as shown in FIG. 6A and FIG. 6B. Taken together, these findings provide important insights into the genetic basis of tumorigenesis across various subtypes of fibroepithelial tumors.

### Example 5

Like FAs, PTs are fibroepithelial tumors, comprising an admixture of epithelial and stromal compartments. To determine the location and distribution of the PT-associated mutations identified in this study, the present disclosure further performed laser capture microdissection (LCM) on 6 PTs from the discovery series. Isolated epithelial and stromal components were analysed separately for mutations in MED12, RARA, FLNA, SETD2, BRCA1 and PIK3CA, the latter two genes being frequently mutated in BCs but less so in PTs (see next paragraph). Briefly, 6 fresh frozen tissues from phyllodes tumors were embedded in Optimal Cutting Temperature (OCT) compound (Tissue-Tek, Sakura Finetek), and sections (8 μm thick) were cut in a Microtome-cryostat (Leica), mounted onto Arcturus® PEN membrane glass slides (Life Technologies), and then stored at −80° C. till required. Slides were dehydrated & stained with Arcturus® Histogene® following manufacturer's recommendations. The stained slide was loaded onto the laser capture microscope stage (ArcturusXT™ Laser Capture Microdissection (LCM) System). A Capsure™ Macro LCM cap (Life Technologies) was then placed automatically over the chosen area of the tissue. Once the cells of interest that were highlighted by the software were verified by the user, the machine automatically dissected out the highlighted cells of interest using a near infrared laser or UV pulse that transferred them onto the Capsure™ Macro LCM Cap. The DNA was extracted directly from LCM caps using Qiagen FFPE DNA Tissue kit following manufacturer's protocol with the following modifications. Each sample cap was incubated with the lysis buffer (ATL & Proteinase K) in a 500 μl microcentrifuge at 60° C. for 5 hrs and enzyme deactivation was carried out at 90° C. for 10 minutes. The eluted DNA was used directly for PCR and Big Dye® sequencing.

The present disclosure found that all of the PT-associated mutations were present in the stromal cells and not in the epithelial cells. These observations are consistent with previous study in FAs where MED12 mutations are detected exclusively in the tumor stroma, suggesting that FAs and PTs likely originate from stromal cells rather than epithelial cells, in spite of their biphasic epithelial-stromal morphological appearance. It is important to note, however, that the present results do not exclude the possibility that genetic alterations may also be present in the epithelial compartment of fibroepithelial tumors. By using OncoCNV analysis, LOH in chrlq was observed in 21% of fibroepithelial tumors in the present disclosure, consistent with a previous study⁴². Furthermore, epithelial alterations are frequently observed in PT⁴³ and histopathological assessment, as in Table 6, indicates that 49% of PTs (32/65, with assessable epithelial compartments) can exhibit moderate-to-florid usual ductal hyperplasia, an epithelial phenotype⁴. It is thus possible that breast fibroepithelial tumor development may involve a complex interplay between the epithelial and stromal compartments of these tumors, warranting further studies.

TABLE 6
Epithelial hyperplasia in phyllodes tumors

| | Number of phyllodes tumors | |
|---|---|---|
| | with assessable epithelial compartment | Percentage |
| hyperlasia | (total number = 65) | (%) |
| Not Aavailable | 10 | 15.4 |
| Mild | 23 | 35.4 |
| Moderate | 28 | 43.1 |
| Florid | 4 | 6.1 |

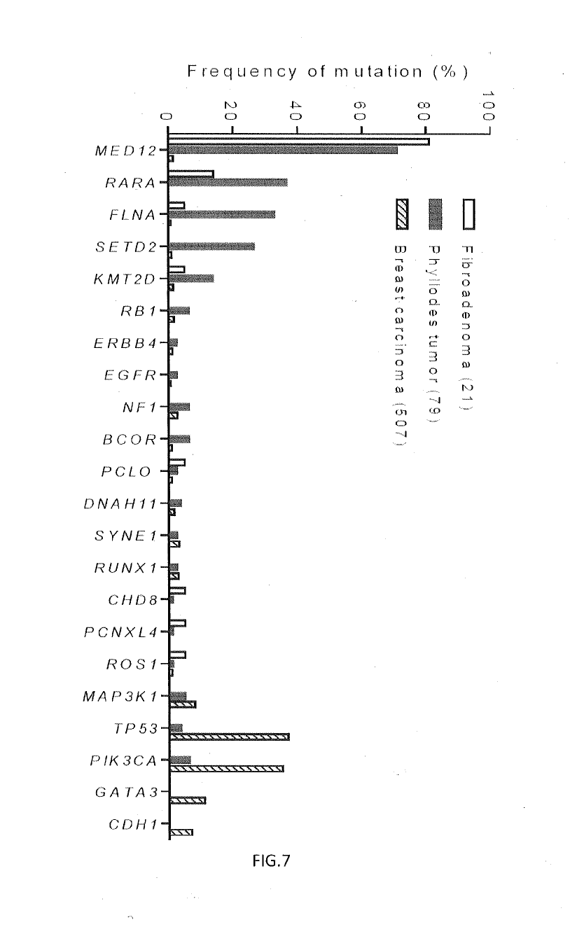

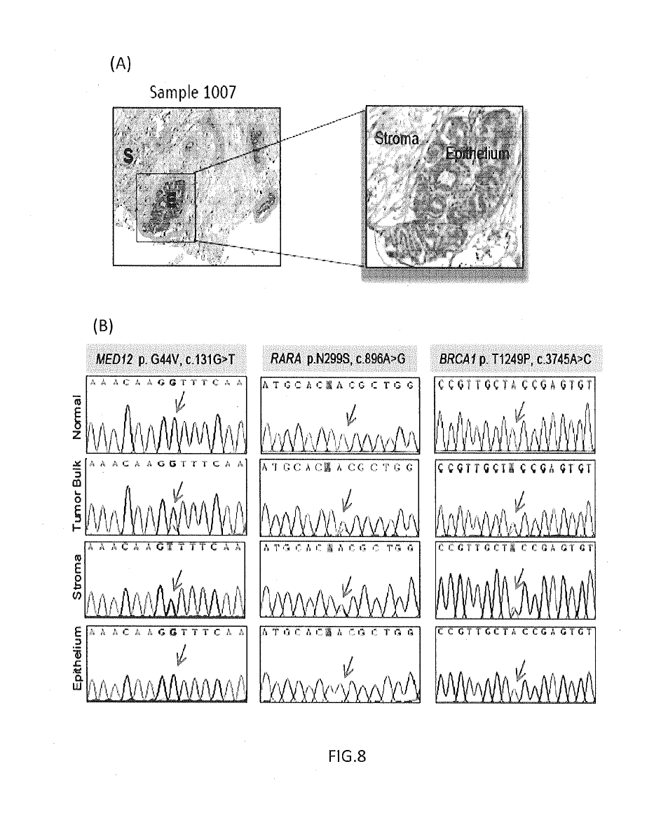

Comparisons between the spectrum and frequency of mutations in fibroepithelial tumors compared to BCs revealed significant distinctions as summarized in FIG. 7. MED12, RARA, FLNA, SETD2 and MLL2 are often mutated in FAs and PTs but are uncommonly mutated in BCs, while TP53, PIK3CA, GATA3 and CDH1 mutations are rare in fibroepithelial tumors but prevalent in BCs. These mutation patterns, as well as the stromal localization of the fibroepithelial-associated driver mutations, indicate distinct molecular pathogenic mechanisms between BCs and breast fibroepithelial tumors, supporting existing guidelines that these two tumors types are distinct diseases entities that should be managed differently.

### Example 6

Full-length RARA cDNAs were cloned into pcDNA3.1 with a 3× Flag tag. The patient-derived mutations were introduced using the QuikChange II XL site-directed mutagenesis kit (Agilent) as described by manufacturer's instructions. The transcriptional activity of wild-type and mutant RARA was assessed by a luciferase assay using the RARE (retinoic acid response element) Cignal reporter assay kit (Qiagen). HEK293 cells were transiently transfected with the RARE reporter construct and Renilla luciferase constructs from the kit, together with the wild-type or mutant RARA plasmids as described above. The transfected cells were then incubated with the indicated concentrations of RA for 24 hours. The luciferase assay was performed using the Dual Luciferase Reporter Assay System (Promega) according to the manufacturer's instructions. Results were normalized to co-expressed Renilla.

For mammalian two-hybrid assays, the RARA ligand binding domain was cloned into pACT vector (Promega) to generate the bait plasmid while the cDNA sequence coding for the CoRNR1 peptide region (THRLITLADHICQIITQDFARNQV) of the NCoR1 protein was inserted into pBIND create the prey plasmid. Mammalian two hybrid screens were carried out with CheckMate Mammalian Two-Hybrid System (Promega) following the manufacturer's protocol. Briefly, transfected HEK293T cells were treated with the indicated concentrations of RA for 24 hours and assayed for luciferase activity. Results were normalized to co-expressed Renilla.

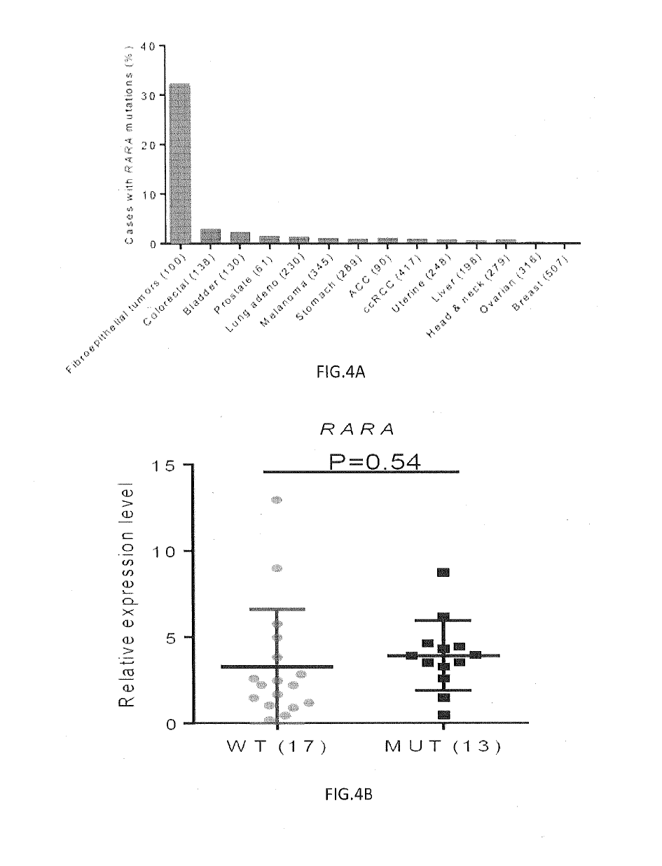

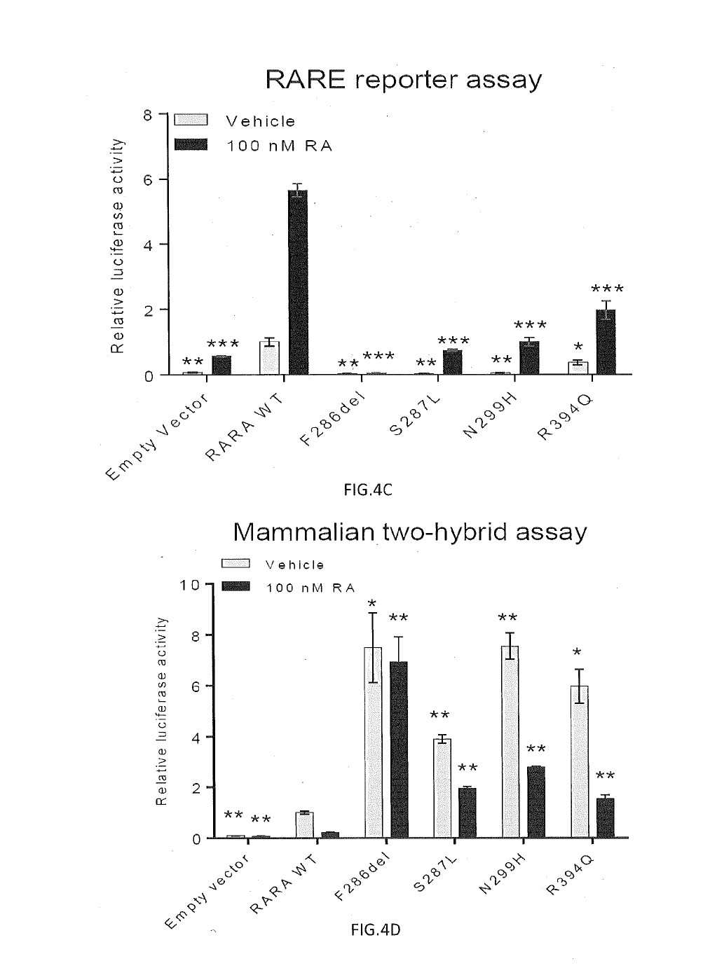

Given the strikingly high frequency of RARA mutations specifically in fibroepithelial tumors as shown in FIG. 4A, present study then proceeded to investigate their functional importance. Previous research has established that RARA is a transcription factor that can interact with co-repressor and co-activator proteins to regulate gene expression. There was no significant difference in RARA expression levels between fibroepithelial tumors harbouring wild-type and mutated RARA genes in view of the results illustrated in FIG. 4B. However, almost all the RARA missense mutations were classified as damaging or deleterious by computational analysis, suggesting that they are biologically consequential. To examine the effects of the RARA mutations on RARA-mediated transcriptional activation, the present disclosure transfected HEK293 cells with a RARE (Retinoic Acid Response Element) reporter construct and cDNA vectors expressing either wild-type RARA or the RARA mutations (F286del, S287L, N299H and R394Q). RARA transcriptional activity was then measured before and after retinoic acid (RA) stimulation. In cells expressing wild-type RARA, stimulation with RA caused a significant increase in RARE-associated transcription. In contrast, cells expressing the mutant forms of RARA exhibited markedly attenuated transcriptional activity, even after RA stimulation as shown in FIG. 4C. The present disclosure hypothesized that the attenuated transcriptional activity of the RARA mutants might be due, at least in part, from these mutations causing enhanced binding of RARA to co-repressor proteins. To test this possibility, the present disclosure used a mammalian two-hybrid assay to probe interactions of both wild-type and mutant RARA proteins with the NcoR1 co-repressor (a known RARA interactor)⁴⁴. In comparison to wild-type RARA, the RARA mutants exhibited higher binding signals to NCoR1 both before and after RA stimulation as being indicated in FIG. 4D, suggesting that the mutant RARA is a more efficient recruiter of co-repressors. Taken together, these results suggest that in breast fibroepithelial tumors, the clustered mutations in RARA may promote the interaction of RARA with co-repressors and hence alter the transcription of RARA target genes.

### Example 7

To confirm the expression of mutant FLNA, the present study also sequenced the cDNA of three FLNA mutant samples with available fresh frozen tissue. One hundred ng of RNA were converted to cDNA with SuperScript III First-Strand Synthesis SuperMix from Invitrogen according to manufacturer's recommended protocol. PCR was performed according to the primers listed in the Table 7. PCR amplification, sequencing and fractionation were performed as described above for Sanger sequencing of genomic DNA.

TABLE 7
Primers used in FLNA cDNA sequencing

| Primer | Forward-sequence 5′ --> 3′ | Reverse-sequence 5′ --> 3′ |
|---|---|---|
| FLNA-A1191 + Y1235 | CTCTTCGCTGACACCCACATCC | TCCACACTGAACTCAGTGGTGG |
| FLNA-G1578 | CCCAGACCGTCAATTATGTGCC | GGGATCTCGTCACCACCGTACT |

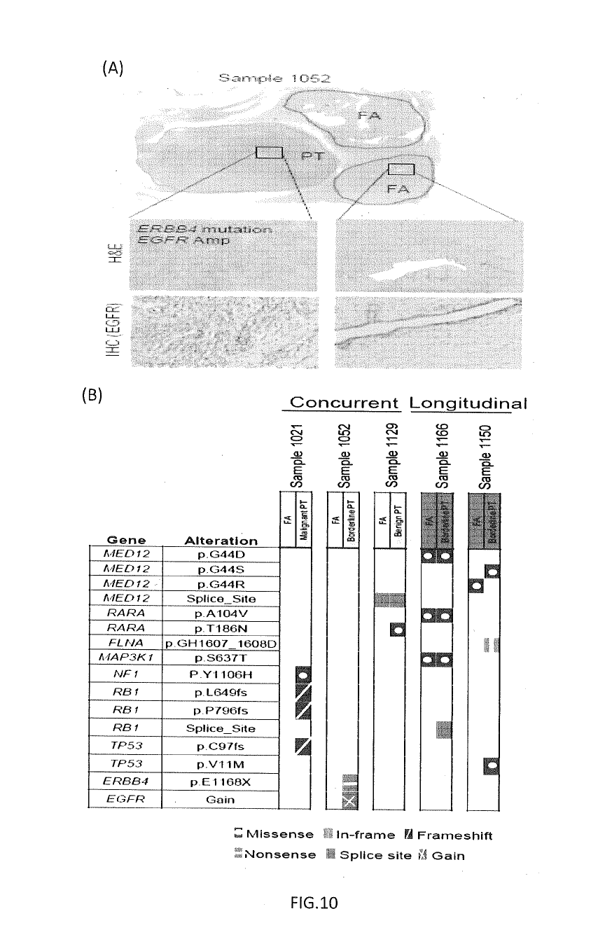

Finally, the present disclosure investigated if FAs might progress to malignant PT in a linear fashion, as proposed in previous studies⁶⁻¹⁰. Using the same targeted 50-gene panel, the experiment sequenced a set of paired concurrent FA and PT-like regions isolated from the same patients (N=3). The present disclosure also analysed paired longitudinal tumors from two patients originally diagnosed with FAs that were subsequently followed by PT-like recurrences. It was found that even in the same patient, higher-grade PTs harboured more mutations than the paired FA regions, especially in cancer-associated genes as indicated in Table 8 and FIG. 10B. Among these patients, two patients, one being concurrent and one being longitudinal, had paired FA and PTs sharing common mutations, consistent with linear progression. However, a third patient (longitudinal) exhibited FA and PT lesions with divergent MED12 mutations, supporting a multifocal origin. The remaining two concurrent cases did not exhibit mutations in the FA and were thus deemed non-informative. Taken collectively, these observations suggest that breast fibroepithelial tumor development may not always follow a strict linear progression model, but may also arise in a multi-focal manner arising from independent lesions in the same breast.

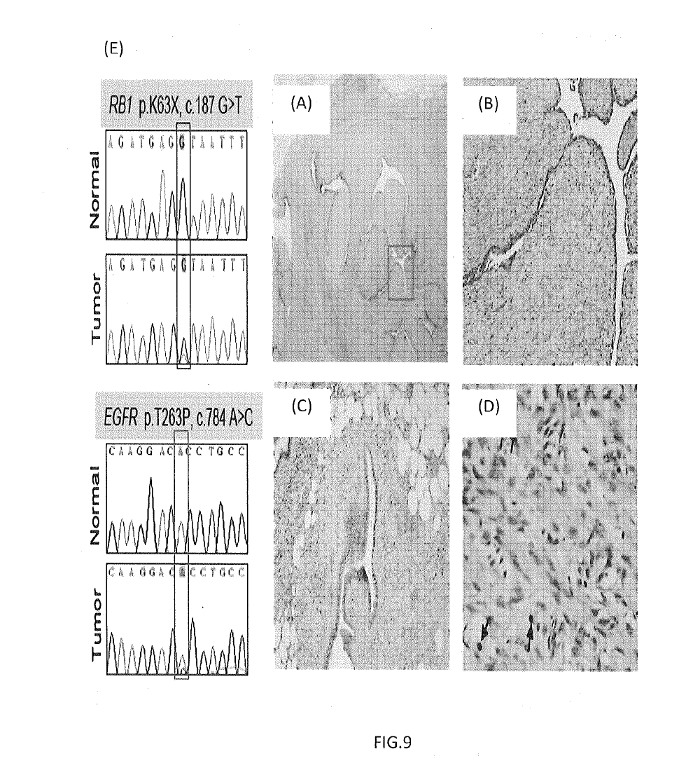

The genomic landscape of breast fibroepithelial tumors described in the present disclosure may have significant clinical implications. As mentioned earlier, the diagnosis and histopathologic classification of PT often present challenges to pathologists. The present disclosure provides the foundation for a genomics-based classification of breast fibroepithelial tumors, which may increase diagnostic accuracy when used in combination with histopathological criteria. For example, based on the acquired sequencing data, Sample 004, previously classified histologically as a benign FA, was found to harbour RB1 truncating and EGFR activating mutations, in addition to MED12, RARA and FLNA mutations referring to FIG. 9, consistent with a borderline/malignant PT signature. This case was subsequently re-evaluated by 2 expert breast pathologists and confirmed to be a borderline PT. Such cases support the notion that ordered mutation profiling may improve the ability to classify fibroepithelial tumors, particularly those associated with malignant status. Beyond diagnosis, the present disclosure also uncovers candidate therapeutic targets for PT. Specifically, canonical activating mutations in PIK3CA and high-level amplifications of EGFR were exclusively found in higher grade PT patients, revealing a potential therapeutic opportunity for EGFR- and PI3K-targeted treatments. This is especially relevant for aggressive malignant PTs, for which there are currently no effective therapeutic options apart from surgery. Also of interest are the mutations affecting MED12 and RARA, which are highly frequent in fibroepithelial tumors and likely influence nuclear hormone receptor signalling^{34,45}. The present experimental data, which establishes for the first time a role for missense RARA mutations in solid tumors, further emphasizes the importance of RARA in fibroepithelial tumors. These genes may thus represent potential therapeutic targets.

Although disclosed method and kit have been described in their preferred form with a degree of particularity, it is understood that the present disclosure of the preferred forms have been made only by way of example and that numerous changes in the details of construction and the combination and arrangements of parts may be resorted to without departing from the scope of the present disclosure.

### REFERENCES

1. Lakhani, S., Ellis, I., Schnitt, S., Tan, P. & Van de Vijver, M. World Health Organisation Classification of tumors of the breast, vol 4., 142-147 (International Agency for Research on Cancer, Lyon, 2012).
2. Lim, W. K. et al. Exome sequencing identifies highly recurrent MED12 somatic mutations in breast fibroadenoma. Nat Genet 46, 877-80 (2014).
3. Coriaty Nelson, Z., Ray, R. M., Gao, D. L. & Thomas, D. B. Risk factors for fibroadenoma in a cohort of female textile workers in Shanghai, China. Am J Epidemiol 156, 599-605 (2002).
4. Tan, P. H. et al. Phyllodes tumors of the breast: the role of pathologic parameters. Am J Clin Pathol 123, 529-40 (2005).
5. Krishnamurthy, S., Ashfaq, R., Shin, H. J. & Sneige, N. Distinction of phyllodes tumor from fibroadenoma: a reappraisal of an old problem. Cancer 90, 342-9 (2000).
6. Hodges, K. B. et al. Evidence for transformation of fibroadenoma of the breast to malignant phyllodes tumor. Appl Immunohistochem Mol Morphol 17, 345-50 (2009).
7. Kuijper, A. et al. Analysis of the progression of fibroepithelial tumours of the breast by PCR-based clonality assay. J Pathol 197, 575-81 (2002).
8. Noguchi, S. et al. Progression of fibroadenoma to phyllodes tumor demonstrated by clonal analysis. Cancer 76, 1779-85 (1995).
9. Kasami, M. et al. Monoclonality in fibroadenomas with complex histology and phyllodal features. Breast Cancer Res Treat 50, 185-91 (1998).
10. Abe, M. et al. Malignant transformation of breast fibroadenoma to malignant phyllodes tumor: long-term outcome of 36 malignant phyllodes tumors. Breast Cancer 18, 268-72 (2011).
11. Cani, A. K. et al. Next-Gen Sequencing Exposes Frequent MED12 Mutations and Actionable Therapeutic Targets in Phyllodes Tumors. Mol Cancer Res 13, 613-9 (2015).
12. Yoshida, M. et al. Frequent MED12 mutations in phyllodes tumours of the breast. Br J Cancer 112, 1703-8 (2015).
13. Piscuoglio, S. et al. MED12 somatic mutations in fibroadenomas and phyllodes tumours of the breast. Histopathology (2015).
14. Nagasawa, S. et al. MED12 exon 2 mutations in phyllodes tumors of the breast. Cancer Med (2015).
15. Jones, A. M. et al. mRNA expression profiling of phyllodes tumours of the breast: identification of genes important in the development of borderline and malignant phyllodes tumours. J Pathol 216, 408-17 (2008).
16. Ang, M. K. et al. Molecular classification of breast phyllodes tumors: validation of the histologic grading scheme and insights into malignant progression. Breast Cancer Res Treat 129, 319-29 (2011).
17. Huang, K. T. et al. DNA methylation profiling of phyllodes and fibroadenoma tumours of the breast. Breast Cancer Res Treat 124, 555-65 (2010).
18. Tan, W. J. et al. Novel genetic aberrations in breast phyllodes tumours: comparison between prognostically distinct groups. Breast Cancer Res Treat 145, 635-45 (2014).
19. Jones, A. M. et al. A comprehensive genetic profile of phyllodes tumours of the breast detects important mutations, intra-tumoral genetic heterogeneity and new genetic changes on recurrence. J Pathol 214, 533-44 (2008).
20. Comprehensive molecular portraits of human breast tumours. Nature 490, 61-70 (2012).
21. Banerji, S. et al. Sequence analysis of mutations and translocations across breast cancer subtypes. Nature 486, 405-9 (2012).
22. Curtis, C. et al. The genomic and transcriptomic architecture of 2,000 breast tumours reveals novel subgroups. Nature 486, 346-52 (2012).
23. Ellis, M. J. et al. Whole-genome analysis informs breast cancer response to aromatase inhibition. Nature 486, 353-60 (2012).
24. Brohl, A. S. et al. The genomic landscape of the Ewing Sarcoma family of tumors reveals recurrent STAG2 mutation. PLoS Genet 10, e1004475 (2014).
25. Lawrence, M. S. et al. Mutational heterogeneity in cancer and the search for new cancer-associated genes. Nature 499, 214-8 (2013).
26. Makinen, N., Vahteristo, P., Butzow, R., Sjoberg, J. & Aaltonen, L. A. Exomic landscape of MED12 mutation-negative and -positive uterine leiomyomas. Int J Cancer 134, 1008-12 (2014).
27. Stephens, P. J. et al. The landscape of cancer genes and mutational processes in breast cancer. Nature 486, 400-4 (2012).
28. Boeva, V. et al. Multi-factor data normalization enables the detection of copy number aberrations in amplicon sequencing data. Bioinformatics 30, 3443-50 (2014).
29. Boeva, V. et al. Control-FREEC: a tool for assessing copy number and allelic content using next-generation sequencing data. Bioinformatics 28, 423-5 (2012).
30. Barbieri, C. E. et al. Exome sequencing identifies recurrent SPOP, FOXA1 and MED12 mutations in prostate cancer. Nat Genet 44, 685-9 (2012).
31. Assie, G. et al. Integrated genomic characterization of adrenocortical carcinoma. Nat Genet 46, 607-12 (2014).
32. Zhu, H. H., Qin, Y. Z. & Huang, X. J. Resistance to arsenic therapy in acute promyelocytic leukemia. N Engl J Med 370, 1864-6 (2014).
33. Kang, Y. K., Guermah, M., Yuan, C. X. & Roeder, R. G. The TRAP/Mediator coactivator complex interacts directly with estrogen receptors alpha and beta through the TRAP220 subunit and directly enhances estrogen receptor function in vitro. Proc Natl Acad Sci USA 99, 2642-7 (2002).
34. Ross-Innes, C. S. et al. Cooperative interaction between retinoic acid receptor-alpha and estrogen receptor in breast cancer. Genes Dev 24, 171-82 (2010).
35. Savoy, R. M. & Ghosh, P. M. The dual role of filamin A in cancer: can't live with (too much of) it, can't live without it. Endocr Relat Cancer 20, R341-56 (2013).
36. Nakamura, F., Osborn, T. M., Hartemink, C. A., Hartwig, J. H. & Stossel, T. P. Structural basis of filamin A functions. J Cell Biol 179, 1011-25 (2007).
37. Zhu, X. et al. Identification of functional cooperative mutations of SETD2 in human acute leukemia. Nat Genet 46, 287-93 (2014).
38. Pasqualucci, L. et al. Analysis of the coding genome of diffuse large B-cell lymphoma. Nat Genet 43, 830-7 (2011).
39. Cimino-Mathews, A. et al. A subset of malignant phyllodes tumors harbors alterations in the Rb/p16 pathway. Hum Pathol 44, 2494-500 (2013).
40. Feakins, R. M., Mulcahy, H. E., Nickols, C. D. & Wells, C. A. p53 expression in phyllodes tumours is associated with histological features of malignancy but does not predict outcome. Histopathology 35, 162-9 (1999).
41. Millar, E. K. et al. Malignant phyllodes tumours of the breast display increased stromal p53 protein expression. Histopathology 34, 491-6 (1999).
42. Sawyer, E. J. et al. Molecular analysis of phyllodes tumors reveals distinct changes in the epithelial and stromal components. Am J Pathol 156, 1093-8 (2000).
43. Jara-Lazaro, A. R. & Tan, P. H. Molecular pathogenesis of progression and recurrence in breast phyllodes tumors. Am J Transl Res 1, 23-34 (2009).
44. Altucci, L. & Gronemeyer, H. The promise of retinoids to fight against cancer. Nat Rev Cancer 1, 181-93 (2001).
45. Markowski, D. N. et al. MED12 mutations in uterine fibroids—their relationship to cytogenetic subgroups. Intl Cancer 131, 1528-36 (2012).
46. Chan-On, W. et al. Exome sequencing identifies distinct mutational patterns in liver fluke-related and non-infection-related bile duct cancers. Nat Genet 45, 1474-8 (2013).
47. Thorvaldsdottir, H., Robinson, J. T. & Mesirov, J. P. Integrative Genomics Viewer (IGV): high-performance genomics data visualization and exploration. Brief Bioinform 14, 178-92 (2013).
48. Saunders, C. T. et al. Strelka: accurate somatic small-variant calling from sequenced tumor-normal sample pairs. Bioinformatics 28, 1811-7 (2012).
49. Sherry, S. T. et al. dbSNP: the NCBI database of genetic variation. Nucleic Acids Res 29, 308-11 (2001).
50. Ng, P. C. & Henikoff, S. SIFT: Predicting amino acid changes that affect protein function. Nucleic Acids Res 31, 3812-4 (2003).
51. Reva, B., Antipin, Y. & Sander, C. Predicting the functional impact of protein mutations: application to cancer genomics. Nucleic Acids Res 39, e118 (2011).
52. Carter, H. et al. Cancer-specific high-throughput annotation of somatic mutations: computational prediction of driver missense mutations. Cancer Res 69, 6660-7 (2009).
53. Choi, Y. & Chan, A. P. PROVEAN web server: a tool to predict the functional effect of amino acid substitutions and indels. Bioinformatics (2015).

1. A method for identifying type of neoplasm in a breast tissue of a subject comprising:
performing one or more nucleic-acid based assays to identify mutations present in the breast tissue acquired from the subject through a first test module and a second test module, each of the first and second test module being associated with detection of at least one predetermined mutation of one or more genes and configured to provide a positive outcome corresponding to at least one predetermined mutation detected in the tissue or a negative outcome corresponding to absence of detectable predetermined mutation in the sample, the first test module being associated with detection of mutation in MED12 gene and/or mutation in RARA gene and the second test module being associated with detection of mutation in FLNA gene, mutation in SETD2 gene and/or mutation in MLL2 gene; and
identifying the type of neoplasm of the breast tissue based upon the provided outcome of the first and second test modules;
wherein the type of neoplasm is regarded as fibroadenomas when the outcome of the first test module and the second test module are respectively positive and negative, and the type of neoplasm is regarded as phyllodes tumor when the outcome of the first test module and the second test module are both positive.

2. The method of claim 1, wherein the performing one or more nucleic-acid based assays further comprising a third test module being associated with detection of mutation in NF1 gene, mutation in RB1 gene and/or mutation in PIK3CA gene.

3. The method of claim 1, wherein the first test module is further associated with detection of mutation in TERT gene of the subject.

4. The method of claim 2, wherein the type of neoplasm is regarded as malignant phyllodes tumor when the outcome of the first test module, the second test module and the third test module are all positive.

5. The method of claim 1, wherein the breast tissue is stromal cells.

6. The method of claim 1, wherein the mutation in MED12 gene is a splice site mutation located at position −8 of exon 2 of the MED12 gene, a missense mutation located at codon 44 of cDNA of the MED12 gene or a missense mutation located at codon 36 of cDNA of the MED12 gene.

7. The method of claim 3, wherein the mutation in TERT gene corresponds to missense mutation located at −124 and/or −146 of a promoter region of the TERT gene.

8. The method of claim 1, wherein the mutation in RARA gene corresponds to p.F286del, p.F287L, p.N299H, p.R394Q, p.L409del and/or p.G289R found in a polypeptide translated thereof.

9. The method of claim 1, wherein the mutation in FLNA gene corresponds to p.A1191T, p.S1199L, p.P1244S, p. 1687-1688TV>M and/or p.S1186W found in a polypeptide translated thereof.

10. The method of claim 1, wherein the mutation in SETD2 gene corresponds to p.R1674-1675EA>D, p.K1587fs, p.Q1545*, p.Y1605fs and/or p.F1651fs found in a polypeptide translated thereof.

11. The method of claim 1, wherein the mutation in MLL2 gene corresponds to p.V5482fs, p.Q1139*, p.G2668fs, p.Q3814* and/or p.L3457fs found in a polypeptide translated thereof.

12. The method of claim 2, wherein the mutation in NF1 gene corresponds to p.K1014*, p.R416* and/or p.D2283fs found in polypeptide translated thereof, the mutation in RB1 gene corresponds to p.Q504*, p.N316fs, and/or p.P796fs found in polypeptide translated thereof and the mutation in PIK3CA gene corresponds to p.H1047R/L found in polypeptide translated thereof.

13. A kit for identifying type of neoplasm in a breast tissue of a subject comprising
at least one platform capable of performing one or more nucleic-acid based assays to identify mutations present in the breast tissue acquired from the subject through a first test module and a second test module, each of the first and second test modules being associated with detection of at least one predetermined mutation of one or more genes and configured to provide a positive outcome corresponding to at least one predetermined mutation detected in the tissue or a negative outcome corresponding to absence of detectable predetermined mutation in the sample, the first test module being associated with detection of mutation in MED12 gene, mutation in TERT gene and/or mutation in RARA gene, the second test module being associated with detection of mutation in FLNA gene, mutation in SETD2 gene and/or mutation in MLL2 gene,
wherein the test modules are configured to emit a detectable or visual signal corresponds to any positive outcome,
wherein the type of neoplasm is regarded as fibroadenomas when the outcome of the first test module and the second test module are respectively positive and negative, and the type of neoplasm is regarded as phyllodes tumor when the outcome of the first test module and the second test module are both positive.

14. The kit of claim 13, wherein the at least one platform further comprising a third test module being associated with detection of mutation in NF1 gene, mutation in RB1 gene and/or mutation in PIK3CA gene.

15. The kit of claim 13, wherein the first test module is further associated with detection of mutation in TERT gene of the subject.

16. The kit of claim 14, wherein the type of neoplasm is regarded as malignant phyllodes tumor when the outcome of the first test module, the second test module and the third test module are all positive.

17. The kit of claim 13, wherein the mutation in MED12 gene is a splice site mutation located at position −8 of exon 2 of the MED12 gene, a missense mutation located at codon 44 of cDNA of the MED12 gene or a missense mutation located at codon 36 of cDNA of the MED12 gene.

18. The kit of claim 13, wherein the mutation in RARA gene corresponds to p.F286del, p.F287L, p.N299H, p.R394Q, p.L409del and/or p.G289R found in a translated polypeptide.

19. The kit of claim 13, wherein the mutation in FLNA gene corresponds to p.A1191T, p.S1199L, p.P1244S, p. 1687-1688TV>M and/or p.S1186W found in a translated polypeptide.

20. The kit of claim 13, wherein the mutation in SETD2 gene corresponds to p.R1674-1675EA>D, p.K1587fs, p.Q1545*, p.Y1605fs and/or p.F1651fs found in a translated polypeptide.

21. The kit of claim 15, wherein the mutation in TERT gene corresponds to missense mutation located at −124 and/or −146 of a promoter region of the TERT gene.

22. The kit of claim 13, wherein the mutation in MLL2 gene corresponds to p.V5482fs, p.Q1139*, p.G2668fs, p.Q3814* and/or p.L3457fs found in a polypeptide translated thereof.
